(19) United States
(12) Patent Application Publication
(10) Pub. No.: US 2013/0276162 A1
(43) Pub. Date: Oct. 17, 2013

(54) Gene Constructs Comprising Nucleic Acids That Modulate Chlorophyll Biosynthesis And Uses Thereof

(71) Applicants: Robert Blankenship, St. Louis, MO (US); Min Chen, Sydney (AU); Robert Willows, Sydney (AU)

(72) Inventors: Robert Blankenship, St. Louis, MO (US); Min Chen, Sydney (AU); Robert Willows, Sydney (AU)

(21) Appl. No.: 13/698,723

(22) PCT Filed: May 20, 2011
(86) PCT No.: PCT/AU2011/000601

§ 371 Date: Apr. 26, 2013

Related U.S. Application Data
(60) Provisional application No. 61/346,743, filed on May 20, 2010.

(51) Int. Cl. C12N 15/82

(52) U.S. Cl. CPC C12N 15/8271
USPC 800/260; 435/320.1; 435/317.1; 435/419; 800/298; 435/468; 800/282; 435/119; 530/387.9

(57) ABSTRACT

The present invention provides isolated nucleic acids encoding Ch1 d synthase, gene constructs comprising the isolated nucleic acids and cells, chloroplasts, plant tissue and whole plants ectopically expressing cyanobacterial Ch1 d synthase. The invention also provides isolated antibodies prepared using recombinant Ch1 d synthase. The antibodies and gene constructs of the invention are used to produce Ch1 d in organisms that do not normally produce Ch1 d, and to modify Ch1 d level in cyanobacterial cells, such as for modifying environmental host range and photosynthetic capacity of organisms in low light and/or red or far-red or near far-red light environments.

### CROSS-REFERENCE TO RELATED APPLICATION

This application claims the benefit of priority from U.S. Application Ser. No. 61/346,743 filed on May 20, 2010, the entire content of which is hereby incorporated by way of reference.

### FEDERALLY-SPONSORED RESEARCH

This invention was produced with the assistance of funding from NASA, USA.

### FIELD OF THE INVENTION

The present invention relates to the fields of agriculture, industrial bioprocessing and diagnostics, and more particularly to the application of molecular technologies for modulating a photosynthetic capacity of an organism and/or for producing detectable fluorescent labels such as for the detection of molecules to which they are attached.

### BACKGROUND TO THE INVENTION

### 1. Photosynthesis and Photosynthetic Pigments

Photoautotrophic organisms such as plants, and algae and bacteria other than those of the kingdom Archeae (e.g., methogenic, halophilic, or thermophilic prokaryotes), are known to produce their own energy source by photosynthetic carbon fixation process(es) wherein carbon dioxide is utilized as a substrate for the biosynthesis of organic compounds, especially sugars. In plants and many algae, the energy to drive such processes is derived from a light source such as sunlight captured by membrane-localized photosynthetic reaction centers i.e., photosystems (PS) such as PSI and PSII, comprising light-harvesting proteins and photosynthetic pigments. In cyanobacteria, the photosynthetic antenna complexes may comprise phycobilisomes and/or phycobiliproteins to serve this function. Briefly, light-dependent reactions or light reactions capture and transfer light energy by resonance energy transfer to a specific pigment or pair of pigments in the reaction center(s), and by virtue of electron transfer reactions from water, hydrogen sulfide or arsenite, produce ATP and NADPH for carbon fixation.

The various pigments required for efficient photosynthesis have different absorption spectral properties, attributable to their structural differences and the proteins with which they are associated in situ. For example, most land plants comprise carotenes, xanthophylls, phaeophytins and chlorophylls, whereas the cyanobacteria may also comprise phycobilins, or proteorhodopsin, the green algae, red algae and glaucophytes may comprise chlorophylls, and some red algae and glaucophytes comprise chlorophylls and phycobilins.

The chlorophylls are Mg-chlorin pigments which vary e.g., by virtue of ligands attached to the Mg²⁺ ion and/or side-chain modifications to the chlorin ring structure and/or a phytol chain. The most widely distributed chlorophyll found in terrestrial plants, algae and cyanobacteria is chlorophyll a. Chlorophylls will have different maximal absorption in vitro depending upon the solvent employed or in vivo. Table 1 below summarizes the different chemical structures, absorption properties (in acetone) and distribution in nature of the most common species of chlorophyll (Ch1). From the absorption properties of the different chlorophylls in acetone (Table 1), it is apparent that Ch1 a absorbs energy from the violet-blue (430 nm) and reddish orange-red (662 nm) regions of visible light, whereas Ch1 b absorbs some energy from blue (457 nm) and predominantly from orange-red (646 nm) light, and Ch1 c absorbs from orange-red light (630-635 nm). In contrast, Ch1 d is the most red-shifted chlorophyll that absorbs in the red and near-infrared regions of the spectrum at about 688 nm and about 25 nm red-shifted relative to chlorophyll a.

TABLE 1

| | Chl a | Chl b | Chl c1 | Chl c2 | Chl d |
|---|---|---|---|---|---|
| Molecular | C₅₅H₇₂O₅N₄Mg | C₅₅H₇₀O₆N₄Mg | C₃₅H₃₀O₅N₄Mg | C₃₅H₂₈O₅N₄Mg | C₅₄H₇₀O₆N₄Mg |
| formula | | | | | |
| C-3 group | CH═CH₂ | CH═CH₂ | CH═CH₂ | CH═CH₂ | CHO |
| C-7 group | CH₃ | CHO | CH₃ | CH₃ | CH₃ |
| C-8 group | CH₂CH₃ | CH₂CH₃ | CH₂CH₃ | CH═CH₂ | CH₂CH₃ |
| C-17 group | CH₂CH₂COO- | CH₂CH₂COO- | CH═CHCOOH | CH═CHCOOH | CH₂CH₂COO- |
| | Phytyl | Phytyl | | | Phytyl |
| C17-C18 bond | Single | Single | Double | Double | Single |
| Absorbance | 430 nm | 457 nm | 635 nm | 630 nm | 447 nm |
| maxima in | 662 nm | 646 nm | | | 688 nm |
| acetone | | | | | |
| Distribution | Widespread | Mostly plants | Various algae | Various algae | Cyanobacteria |

The structures of Ch1 a and Ch1 d are set forth in FIG. 1.

Swingley et al., Proc. Natl. Acad. Sci. (USA) 105, 2005-2010 (2008) disclose Ch1 d as the major photosynthetic pigment in the cyanobacterium Acaryochlorus marina, and suggest that Ch1 d is most likely synthesized from Ch1 a or chlorophyllide a, however that any such synthesis would be complex by virtue of a requirement inter alia to derive the formyl group at C-3 in Ch1 d from a vinyl group at this position in Ch1 a, possibly requiring multiple enzymes to achieve two-step oxidative cleavage of the double bond. The authors suggest several alternative families of proteins that might achieve Ch1 d synthesis in A. marina, including a putative Ch1 d synthase similar to chlorophyllide a oxygenase (CAO) comprising a CAO-type Rieske-FeS motif, and a putative protein similar to divinyl chlorophyllide reductase, in addition to a conceptually distinct mechanism that requires a putative radical SAM enzyme i.e., comprising a radical SAM motif that transfers an oxygen free radical from S-adenosyl methionine (SAM). However, the source of formyl oxygen at C-3 in Ch1 d is not known and, as a consequence, it is not possible to determine which of the possibilities described by Swingley et al. is correct. The complete genome sequence of A. marina, and annotations for candidate Ch1 d synthase-encoding genes are also disclosed by Swingley et al., Proc. Natl Acad. Sci. (USA) 105, 2005-2010 (2008).

Yang et al., Biochimica et Biophysica Acta 1797, 204-211 (2010), incorporated herein in its entirety by way of reference, have demonstrated that the A. marina gene encoding a chlorophyll d-binding light-harvesting protein (syn. accessory Chlorophyll Binding Protein, abbreviated CBPII; pcbA gene Accession No. AMI_{—}3654) is expressed and integrates into the thylakoid membranes of Synechocystis PCC 6803, where it binds preferentially to Ch1 a. Those data indicate that A. marina CBPII interacts with photosystem II (PSII) of Synechocystis PCC 6803, and suggest that the thylakoid membranes of higher plants will also integrate ectopically-expressed A. marina CBPII-encoding gene that functions to bind higher plant Ch1 a. Yang et al., (2010) suggest that photosynthetic organisms have the capacity to take advantage of any particular light-harvesting Ch1 that they are able to synthesize.

### 2. Plant Productivity and Industrial Bioprocessing

Light intensity and light quality i.e., the color or wavelength reaching the plant surface, affect plant productivity. For example, red and blue light have a greater effect on enhancing plant growth compared to green light, and blue light appears to be primarily responsible for enhancing vegetative growth or leaf expansion, and the combination of red light and blue light may enhance flowering in plants. Fluorescent or cool-white light, which is high in blue light, is known to encourage leafy growth. Terrestrial Ch1 a+b plants grown in low light environments or environments which are otherwise deficient in red and/or near-infrared light, such as occurs beneath forest canopies or densely grown crops, have reduced productivity characterized e.g., by stem elongation at the expense of leaf expansion, and/or by a reduced seed yield. Thus, organisms comprising Ch1 a alone or Ch1 a+b or Ch1 a+c, which do not absorb light efficiently from the red or near infrared regions of the visible light spectrum, are less efficient at maintaining productivity in low light environments than organisms comprising Ch1 d. In contrast, cyanobacteria e.g., Acaryochlorus marina are normally found in low-light environments in which terrestrial plants do not normally grow, or grow poorly (Kuhl et al., Nature 433, 820 (2005). By using Ch1 d, cyanobacteria thrive in environments characterized by low, visible light intensity and a high near-infrared intensity e.g., Swingley et al., Proc. Nail Acad. Sci. (USA) 105, 2005-2010 (2008).

Light quality, especially the relative amounts of blue, red and near far red light that reach the biosphere, also varies seasonally, such that there is more blue light in longer days than shorter days. This accounts for increased vegetative growth during spring and early summer. As the day length shortens, and the relative amount of red and far red light increases e.g., during late summer and fall, vegetative growth is reduced.

Notwithstanding the well-known commercial utilities of photoautotrophic organisms e.g., as feed, feedstock, and as sources of valuable secondary metabolites, the dependence of such organisms on light intensity and/or light quality for maximum yield is a variable that affects the profitability of industrial bioprocesses e.g., use of plants, algae and cyanobacteria as feed for humans and other animals or for downstream processing to produce biofuels, oils, nutraceuticals, specialty chemicals, pharmaceuticals, etc. In particular, most photoautotrophic algae and cyanobacteria have long doubling times in culture compared to heterotrophic bacteria e.g., Escherichia coli or yeasts.

There remains a need for photoautotrophic organisms e.g., that are less susceptible to environmental factors such as light intensity and/or light quality, and/or that have an ability to harvest light of different quality, compared to their naturally-occurring counterparts.

### 3. Fluorescent Tags

Fluorescent molecules are known to be used as labels, which by virtue of their easy identification, are useful in diagnostics e.g., to label detector molecules in assays. There is a need in agriculture and industrial bioprocessing for inexpensive fluorophores that can readily be introduced into a field context for identifying or tracking living organisms and/or cells, especially in the context of agricultural crops or downstream processes thereof e.g., seed production. For example, it is desirable in the seed and agriculture industries to identify seed and plants expressing particular traits and/or to monitor their spread. It is highly desirable to provide such a fluorescent tag by recombinant means e.g., by engineering expression of the tag into an organism, to thereby permit identification of progeny cells or organisms from parental line(s).

Fluorescein and rhodamine are commonly used fluorophores. Fluorescein is readily available in an activated form for direct coupling to antigens or antibodies. Both fluorescein and rhodamines show good chemical stability and have a proven record in actual use as labels. However, both fluorescein and the rhodamines have relatively small Stokes shifts, which limits their applicability. Umbelliferones and lanthanide chelates have larger Stokes shifts than fluorescein and rhodamine, but the coupling between lanthanide chelates and antigens is problematic, and the fluorophore itself can be labile, restricting its utility. Because these compounds are synthetically produced, they are not normally amenable to being produced by recombinant means or capable of permitting the identification of progeny cells or organisms from parental line(s).

Proteinaceous fluorophores based on phycobiliproteins are described in U.S. Pat. Nos. 4,520,110 and 4,542,104. A phycobiliprotein tandem conjugate between phycoerythrin and allophycocyanin has a large Stokes shift with an emission maximum at 660 nm and an excitation waveband that starts at about 440 nm, however the production of the conjugate is complex. Since most terrestrial plants do not possess the cellular enzymes necessary to produce phycoerythrin or allophycocyanin, the production of such compounds by plants is difficult and may require the introduction of whole biosynthetic pathways into plants, for the purpose of tracking cells or whole organisms across generations. Moreover, even if such recombinant production was readily achievable, the subsequent production in vivo of tandem conjugates between phycoerythrin and allophycocyanin is unlikely.

Fluorescent tags comprising peridinin-chlorophyll a-proteins (PerCPs) have been described in U.S. Pat. No. 4,876,190. Because the choice of a fluorophore for any particular application is constrained, at least in part, by the ability of cells or ligands associated with such compounds to fluoresce, or to contain compounds that fluoresce, under the same conditions as a fluorescent tag, thereby masking the signal generated by the tag, PerCPs have limited application in agriculture. For example, PerCPs are susceptible to masking by endogenous Ch1 a in photosynthetic organisms that normally produce that pigment e.g., most photosynthetic organisms other than cyanobacteria.

Accordingly, there remains a need for fluorescent labels capable of being produced readily by the cellular machinery of a Ch1 a-containing organism that is not masked by endogenous pigments of the organism.

### SUMMARY OF THE INVENTION

In work leading up to the present invention, the inventors sought to characterize the pathway for Ch1 d biosynthesis using the model cyanobacterium A. marina, and to ascertain the class of proteins to which Ch1 d synthase belongs. As exemplified herein, ¹⁸O labelling of Ch1 a and Ch1 d indicates that Ch1 a is a biosynthetic precursor of Ch1 d, and the formyl oxygen at C-3 of Ch1 d is derived from atmospheric oxygen by the action of a cytochrome P450 dioxygenase enzyme.

The inventors have screened a family of putative A. marina cytochrome P450 oxygenases expressed in the bacterium Escherichia coli from amplified synthetic gene fragments, for activity under different gasification environments. The inventors also developed a fluorescence-based assay for determining Ch1 d biosynthesis by E. coli, wherein bacterial cells having the ability to ectopically express a Ch1 d synthase can convert Ch1 a to Ch1 d at room temperature in the dark. Using this fluorescence-based assay, the inventors have shown that E. coli transformed with a genetic construct comprising nucleic acid of the A. marina AMI_{—}3563 locus (SEQ ID NO: 11) placed operably under the control of a suitable promoter express a Ch1 d synthase of the cytochrome P450 oxygenase family of proteins (SEQ ID NO: 12) that is capable of converting Ch1 a into Ch1 d at room temperature in the dark. The data herein also indicate that the Ch1 d synthase of A. marina is functional across different kingdoms.

The inventors have also isolated sequence variants of the Ch1 d synthase-encoding nucleic acid set forth in SEQ ID NO: 11 from geographical isolates of A. marina obtained from different marine environments in Australia. The sequences of these variant Ch1 d synthase-encoding nucleic acids are set forth in SEQ ID Nos: 21, 23, 25 and 27, and the corresponding encoded amino acid sequences are set forth in SEQ ID Nos: 22, 24, 26 and 28 respectively.

The inventors have also produced antibodies that bind to Ch1 d synthase polypeptide.

The provision by the present inventors of A. marina Ch1 d synthase-encoding nucleic acid, and the encoded Ch1 d synthase protein, provide the means for producing gene constructs for modifying chlorophyll biosynthesis in vivo in a number of different organisms, that ordinarily produce at least Ch1 a, and/or for producing Ch1 d in any organism that otherwise does not produce this pigment. The present invention also provides a significant benefit in so far as the Ch1 d synthase-encoding nucleic acid and gene constructs comprising same can be employed to modify, e.g., enhance or up-regulate, Ch1 d synthesis and levels in Ch1 d-containing organisms such as cyanobacteria.

### 1. Specific Examples

Accordingly, one example of the present invention provides an isolated Ch1 d synthase gene comprising a sequence selected from the group consisting of:
(i) the sequence set forth in SEQ ID NO: 11 or a variant thereof comprising a sequence that is degenerate with SEQ ID NO: 11 by virtue of the genetic code and/or that varies from SEQ ID NO: 11 by virtue of a codon usage bias;
(ii) a sequence comprising an open reading frame that encodes the amino acid sequence set forth in SEQ ID NO: 12 or a variant thereof comprising a sequence wherein one or more amino acids of SEQ ID NO: 12 is substituted conservatively for one or more other amino acids in said variant sequence;
(iii) a sequence that is produced by amplification employing one or more primer sequences comprising SEQ ID NO: 14 and SEQ ID NO: 15;
(iv) a sequence having at least about 80% sequence identity to SEQ ID NO: 11;
(v) a sequence comprising an open reading frame that encodes an amino acid sequence having at least about 80% identity to the amino acid sequence set forth in SEQ ID NO: 12;
(vi) a sequence that is produced by amplification employing one or more primer sequences comprising SEQ ID NO: 14 and SEQ ID NO: 15;
(vii) a sequence that is produced by amplification employing primer sequences comprising SEQ ID NO: 18 and SEQ ID NO: 20 or primer sequences comprising SEQ ID NO: 19 and SEQ ID NO: 20;
(viii) a sequence that hybridizes under at least moderate stringency conditions to a sequence that is complementary to (i) or (ii) or fragment thereof comprising at least 10 nucleotides in length; and
(ix) a sequence that is complementary to a sequence at (i) or (ii) or (iii) or (iv) or (v) or (vi) or (vii) or (viii).

As used herein, the term “Ch1 d synthase gene” shall be taken to include a structural gene e.g., a protein-encoding part of a genomic gene, that comprises sufficient nucleotide sequence to encode a polypeptide that is functional in producing Ch1 d in a cell or organelle such as a chloroplast, or in a cell part e.g., thylakoid membrane. For example, a Ch1 d synthase gene of the present invention may comprise all of SEQ ID NO: 11 or a variant thereof as described, or alternatively, a functional fragment of SEQ ID NO: 11 or said variant e.g., wherein one or more nucleotides have been deleted from the 5′-end and/or 3′-end of the base sequence without adversely affecting the ability of the encoded polypeptide to catalyse Ch1 d synthesis e.g., from Ch1 a. It will also be apparent from this definition that the term “Ch1 d synthase gene” includes a genomic gene equivalent of SEQ ID NO: 11 or a variant thereof as described e.g., comprising one or more regulatory sequences to which SEQ ID NO: 11 or said variant is operably linked in nature, including a promoter sequence and/or a transcriptional terminator sequence or other transcriptional regulatory sequence. Preferred regulatory sequences of a Ch1 d synthase gene of the present invention confer or enhance or otherwise regulate transcription and/or stability and/or turnover of Ch1 d synthase gene mRNA in one or more of a cell, nucleus or plastid.

The term “degenerate” is understood to mean that a nucleotide sequence encodes the same amino acid sequence as a reference nucleotide sequence, by virtue of the genetic code providing a plurality of codons encoding the same amino acid residue i.e., synonymous codons or functionally-equivalent codons. Such degenerate sequences include those that arise by virtue of a codon usage bias of an organism e.g., that optimizes translation. For example, a codon preference may reflect the composition of a tRNA pool such that mRNAs comprising codons that accept tRNAs of the tRNA pool are translated more efficiently and/or at higher accuracy than those that do not, thereby effecting expression. For example, a variant of SEQ ID NO: 11 for expression in a chloroplast may comprise one or more nucleotide substitutions such that one or more codons ends with A or T or U. In another example, a variant of SEQ ID NO: 11 for expression in a cyanobacterium or from the nuclear genome of green algae may comprise one or more nucleotide substitutions such that one or more codons ends in C or G. In another example, a variant of SEQ ID NO: 11 for expression from the nuclear genome of a monocotyledonous plant may comprise one or more nucleotide substitutions such that one or more codons ends in C or G. In another example, a variant of SEQ ID NO: 11 for expression from the nuclear genome of a dicotyledonous plant may comprise one or more nucleotide substitutions such that one or more codons ends in A or T or U.

By “substituted conservatively” is meant that the nucleotide sequence of the Ch1 d synthase gene comprises one or more substitutions relative to SEQ ID NO: 11 such that one or more encoded amino acids of SEQ ID NO: 12 is substituted for another amino acid of similar charge and/or polarity and/or hydrophilicity, without ablating Ch1 d synthase function notwithstanding that one or more kinetic parameters of the enzyme may be modified, e.g., Gly [symbol] Ala, Val [symbol] Ile [symbol] Leu, Asp [symbol] Glu, Lys [symbol] Arg, Asn [symbol] Gln or Phe [symbol] Trp [symbol] Tyr.

As used herein, the term “amplification” shall be taken to mean any nucleic acid sequence-based amplification (NASBA) e.g., an isothermal amplification or other amplification not requiring thermal cycling, or an amplification reaction requiring thermal cycling such as a polymerase chain reaction (PCR) including standard PCR, reverse-transcriptase mediated PCR (RT-PCR).

The term “at least moderate stringency” shall be taken to mean other than a low stringency hybridization according to standard protocols known to the skilled artisan. For example, a moderate stringency may comprise an incubation temperature between about 42° C. and about 55° C. and/or an incubation temperature between about 15° C. and 10° C. lower than a predicted melting temperature (Tm) of a reference sequence e.g., SEQ ID NO: 11 or variant or a complementary sequence thereto. In another example, a high stringency may comprise an incubation temperature between about 55° C. and about 65° C. and/or an incubation temperature between about 10° C. and 1° C. lower than a predicted melting temperature (Tm) of a reference sequence e.g., SEQ ID NO: 11 or variant or a complementary sequence thereto.

Another example of the present invention provides a gene construct comprising a Ch1 d synthase gene and one or more origins of replication for maintenance of the gene construct in a cell or chloroplast, said Ch1 d synthase gene comprising a sequence selected from the group consisting of:
(i) the sequence set forth in SEQ ID NO: 11 or a variant thereof comprising a sequence that is degenerate with SEQ ID NO: 11 by virtue of the genetic code and/or that varies from SEQ ID NO: 11 by virtue of a codon usage bias;
(ii) a sequence comprising an open reading frame that encodes the amino acid sequence set forth in SEQ ID NO: 12 or a variant thereof comprising a sequence wherein one or more amino acids of SEQ ID NO: 12 is substituted conservatively for one or more other amino acids in said variant sequence;
(iii) a sequence that is produced by amplification employing one or more primer sequences comprising SEQ ID NO: 14 and SEQ ID NO: 15;
(iv) a sequence that hybridizes under at least moderate stringency conditions to a sequence that is complementary to (i) or (ii) or fragment thereof comprising at least 10 nucleotides in length; and
(v) a sequence that is complementary to a sequence at (i) or (ii).

As used herein, the term “gene construct” is to be construed broadly to include e.g., a plasmid, phagemid, cosmid, viral genome or subgenomic fragment, phage artificial chromosome e.g., P1 artificial chromosome, a bacterial artificial chromosomes (BAC), a yeast artificial chromosome (YAC), or other nucleic acid capable of being maintained chromosomally or extra-chromosomally and/or replicating in a cell.

Those skilled in the art are aware that the term “origin of replication” in the context of a gene construct means nucleic acid e.g., DNA or ssRNA or dsRNA, comprising a sequence at which replication is initiated, to thereby maintain the copy number of the gene construct in a cell by virtue of replication proceeding from the point of origin e.g., bidirectionally or unidirectionally.

In another example, a gene construct according to any example hereof comprises one or more sequences to permit it to be maintained in a cell under selective conditions e.g., one or more selectable marker genes.

In another example, a gene construct according to any example hereof further comprises one or more 5′ non-coding regions. As used herein, the term “5′ non-coding region” shall be taken in its broadest context to include any nucleotide sequence derived from the upstream region of a gene, e.g., a promoter, an intron, e.g., an intron derived from a ubiquitin gene, a cis-regulatory region e.g., that is a functional binding site for a transcriptional regulatory protein or translational regulatory protein and/or one or more upstream activator sequences, enhancer elements or silencer elements and/or an upstream open reading frame (uORF). As used herein, the term “uORF” refers to a nucleotide sequence localized upstream of a functional translation start site in a gene and generally within the 5′-transcribed region e.g., a leader sequence. Whilst not being bound by any theory or mode of action, a uORF functions to prevent over-expression of a structural gene sequence to which it is operably connected or alternatively, to reduce or prevent such expression.

In another example, a gene construct according to any example hereof comprises a promoter operably connected to the Ch1 d gene or to a selectable marker gene such to thereby provide for expression of the gene e.g., as mRNA and/or as protein. As used throughout this specification and in the claims that follow, the terms “operably connected” and “in operable connection with” mean the positioning of a functional nucleic acid unit e.g., a promoter or transcription terminator or active fragment or derivative thereof, in spatial relation to another nucleic acid unit, e.g., a Ch1 d synthase transgene or fragment thereof, to thereby permit the units to function in concert such as by a promoter conferring expression on a transgene. A promoter will generally be positioned 5′ (upstream) of a Ch1 d synthase gene to which it is operably connected. Exemplary promoters are described herein.

In yet another example, a gene construct according to any example hereof comprises one or more intron sequences and/or intron splice junction sequences e.g., e.g., from a ubiquitin gene, placed in operable connection with the Ch1 d synthase gene e.g., to thereby enhance nuclear mRNA stability and/or processing and/or translation of nuclear mRNA in a eukaryotic cell. An intron sequence and intron splice junction sequence is generally selected from a species compatible with mRNA processing in a cell in which the Ch1 d synthase is expressed. Exemplary introns are described herein.

In another example, a gene construct according to any example hereof further comprises a sequence operably connected to the Ch1 d gene to thereby provide for transcription termination, and optionally, polyadenylation of mRNA. Exemplary terminators are described herein.

In yet another example, a gene construct according to any example hereof comprises a sequence encoding a targeting sequence or a detectable label e.g., positioned the promoter and the Ch1 d synthase gene such that it is expressed as a 5′-fusion with Ch1 d synthase. Alternatively, the additional component may be located 3′ to the Ch1 d synthase gene such that it is expressed as a 5′-fusion with Ch1 d synthase. For example, the Ch1 a′ synthase may be expressed as a fusion polypeptide with one or more of detectable markers as described herein.

In yet another example, a gene construct according to any example hereof comprises a targeting sequence that encodes a peptide that directs the Ch1 d synthase polypeptide to a particular subcellular location e.g., a chloroplast or thylakoid membrane. Exemplary targeting sequences are described herein.

In yet another example, a gene construct according to any example hereof comprises one or more recombinase site sequences to permit excision of a portion of its DNA in a cell and/or to facilitate integration into host cell DNA e.g., chloroplast DNA or genomic DNA. Exemplary recombinase site sequences are described herein.

In the case of a gene construct to be delivered to the nuclear genome of algae or plants e.g., dicotyledonous plants, by Agrobacterium-based transformation, the vector preferably comprises a left-border (LB) sequence and a right-border (RB) sequence that flank a transgene comprising the Ch1 d synthase gene and any additional sequence(s) to which it is operably connected, e.g., to facilitate transfer DNA. Such a vector may also comprise a suitable selectable marker for selection of bacteria comprising the vector, e.g., conferring resistance to ampicillin.

In another example, a gene construct according to any example hereof comprises means for introducing elements in operable connection with each other e.g., one or more multiple cloning sites each comprising one or more restriction endonuclease cleavage site(s), and/or one or more recombination site(s).

It is to be understood that a gene construct of the present invention may comprise any one or more elements as described according to any example hereof, in any combination unless the context requires otherwise, subject to the requirement for the Ch1 d synthase gene of the invention and one or more origins of replication.

In yet another example, the gene construct of the present invention is an expression construct that provides for transcription of mRNA encoding a functional Ch1 d synthase polypeptide or a functional fragment thereof in the nucleus or chloroplast of a eukaryote and/or in a prokaryotic cell. Alternatively, or in addition, the gene construct of the present invention may be an expression construct that provides for translation of a functional Ch1 d synthase polypeptide or a functional fragment thereof in a chloroplast or prokaryotic cell. For example, the gene construct is suitable for expression of the Ch1 d synthase gene of the invention in a prokaryotic cell selected from a bacterial cell e.g., E. coli, and/or a cyanobacterium e.g., A. marina, A. nidulans. Alternatively, the gene construct is suitable for expression of the Ch1 d synthase gene of the invention in a eukaryotic cell e.g., a plant or algae. Alternatively, the gene construct is suitable for expression of the Ch1 d synthase gene of the invention in a chloroplast of a plant or algae. As will be known to the skilled artisan, a minimum requirement for such expression is that the Ch1 d synthase gene of the invention is linked operably to a promoter or regulatory sequence capable of conferring expression in a cell of interest.

In yet another example, the gene construct of the present invention is a shuttle vector that is able to be maintained and/or replicate in at least two different host organisms e.g., E. coli and S. cerevisiae, or a bacterium and a cyanobacterium, or a bacterium and a plant, or a cyanobacterium and a plant. A shuttle vector in the present context also includes a gene construct that able to be maintained and/or replicate in a chloroplast and any one or more of a bacterium, a cyanobacterium, an algae and a plant. Such vectors facilitate introduction of the Ch1 d synthase gene construct of the invention into different species.

In yet another example, the gene construct of the present invention is a binary Ti plasmid or Ri plasmid.

In one preferred example, a gene construct according to the present invention comprises structural features suitable at least for expression of the Ch1 d synthase gene of the present invention in Synechocystis sp. PCC 6803. For example, the gene construct may comprise substantially the same backbone elements as the vector designated pWS19K described by Yang et al., Biochimica et Biophysica Acta 1797, 204-211 (2010), incorporated herein in its entirety by way of reference. In this example, the Ch1 a′ synthase-encoding gene, optionally comprising a sequence encoding a 3′-terminal marker such as a hexahistidine tag for expression as an in-frame fusion with Ch1 d synthase, is positioned between pcbAIII gene promoter and transcription termination signals, such that expression of the Ch1 d synthase-encoding gene is operably regulated by the pcbAIII promoter. For example, The pcbAIII (sIII867) upstream region (527 bp) may be amplified by PCR from plasmid pWS19K, and joined to nucleic acid encoding Ch1 d synthase to thereby produce an integrating product comprising both the pcbAIII upstream region and Ch1 d synthase-encoding gene, which is then introduced into plasmid pWS19K upstream of the endogenous pcbA ill transcription terminator of the vector and replacing the endogenous pcbAIII upstream region of the vector. The recombined vector pSW19K is then transformed into Synechocystis PCC 6803 and the transformed cells are obtained and cultured.

In another preferred example, a gene construct according to the present invention comprises structural features suitable at least for expression of the Ch1 d synthase gene of the present invention in a higher plant e.g., a monocotyledonous plant or dicotyledonous plant. For performing this example, the skilled artisan will know to position the Ch1 d synthase-encoding gene in operable connection with a promoter element and transcription terminator operable in the target organism, optionally wherein the construct comprises an intron-1 sequence positioned after the transcription start site of the structural gene and a chloroplast translocation-encoding sequence to permit translocation of the encoded protein to the chloroplast. The gene construct(s) will generally also comprise one or more origins of replication, and one or more selectable marker genes, and optionally, one or more Agrobacterium integration-specific elements e.g., left and right border sequences to facilitate Agrobacterium-mediated integration where appropriate.

The present invention clearly provides a gene construct comprising structural features suitable at least for expression of the Ch1 d synthase gene of the present invention according to any example hereof in combination with a gene construct comprising structural features suitable at least for expression of the A. marina CBPII protein. Without being bound by any theory or mode of action, co-expression of A. marina Ch1 d synthase and CBPII proteins provides a means for enhancing the ability of a transformant organism expressing the Ch1 d synthase of the present invention to produce Ch1 d in vivo and incorporate the Ch1 d into a thylakoid membrane such that the incorporated Ch1 d functions in photosynthesis. As will be known to the skilled artisan, the gene constructs encoding Ch1 d synthase and CBPII may be combined into a single plasmid or other vector, or alternatively, be maintained on separate plasmids or other vectors. As will be apparent from the description provided herein, the specific structure of the gene construct(s) employed will vary by art-recognized substituents e.g., selected from promoter(s), terminator(s), origin(s) of replication, intron sequence(s), protein targeting domain-encoding sequence(s), transit peptide-encoding sequence(s), chloroplast translocation-encoding sequence(s), selectable marker gene(s), and Agrobacterium integration-specific element(s), depending upon the target organism e.g., higher plant such as a monocotyledonous plant or dicotyledonous plant, green algae, or cyanobacterium such as Synechocystis PC 6803, Thus, the Ch1 d and CBPII protein may be co-expressed such that they are functional, and preferably interact, such as in the PSII of a higher plant, green algae or cyanobacterium such as Synechocystis PC 6803. In performing this example, of the invention in Synechocystis sp., PC6803, it is particularly preferred to utilize a gene construct described herein for expressing the A. marina Ch1 d synthase-encoding gene in that organism in combination with the gene construct designated pWS19k-pcbA as described by Yang et al., Biochimica et Biophysica Acta 1797, 204-211 (2010), incorporated herein in its entirety by way of reference. Alternatively, a functionally-equivalent structural variant of pWS19k-pcbA may be employed. In performing this example in a higher plant e.g., a monocotyledonous plant or dicotyledonous plant, the skilled artisan will know to position each of the Ch1 d synthase and CBPII genes in operable connection with a promoter element and transcription terminator operable in the target organism, optionally wherein each construct comprises an intron-1 sequence positioned after the transcription start site of the structural gene and a chloroplast translocation-encoding sequence to permit translocation of the encoded protein to the chloroplast. Preferably, promoters driving expression of Ch1 d synthase and CBPII genes will be different e.g., to minimize squelching effects in the transformed cell, Depending upon whether or not the gene constructs are expressed from one or two plasmids or other vectors, the gene construct(s) will generally also comprise one or more origins of replication, and one or more selectable marker genes, and optionally, one or more Agrobacterium integration-specific elements e.g., left and right border sequences to facilitate Agrobacterium-mediated integration where appropriate.

In another example, the present invention also provides for use of a Ch1 d synthase gene as described according to any example hereof in the preparation of a gene construct for expressing a Ch1 d synthase in a cell or chloroplast. Optionally, this example further comprises use of the A. marina CBPII gene as described herein in the preparation of a gene construct for co-expressing CBPII in combination with the Ch1 d synthase of the invention.

In a related example, the present invention also provides a method for producing a gene construct, said method comprising linking a promoter or active fragment or derivative thereof to a Ch1 d synthase gene as described according to any example hereof such that the promoter confers expression or a pattern of expression on said gene in a cell or chloroplast, optionally in combination with A. marina CBPII. Preferred cells are bacterial cells, cyanobacterial cells, algal cells and plant cells e.g., crop plants such as wheat, barley, maize, rice, sorghum, rye, millet (e.g. pearl millet or proso millet), buckwheat (e.g., of the family Polygonaceae), oat (e.g., Avena saliva), tomato, spinach, oilseed rape, etc.

In another example, the method additionally comprises producing or obtaining a Ch1 d synthase gene as described according to any example hereof.

A further example of the present invention provides a cell comprising a gene construct of the present invention as described according to any example hereof or a fragment of a gene construct comprising a Ch1 d synthase gene, e.g., a bacterial cell, cyanobacterial cell, yeast cell, algal cell or plant cell. In this respect, the gene construct or fragment thereof comprising a Ch1 d synthase gene, optionally in combination with a gene construct comprising the A. marina CBPII-encoding gene pcbA, may be present as an extrachomosomal genetic element or episome in the cell e.g., as a plasmid or cosmid, especially in the case of a prokaryotic cell. Alternatively, the gene construct or fragment thereof comprising a Ch1 d synthase gene, optionally in combination with a gene construct comprising the A. marina CBPII-encoding gene pcbA, may be integrated into a genome of the cell e.g., a nuclear genome or chloroplast genome of the cell.

A further example of the present invention provides a chloroplast comprising a gene construct of the present invention as described according to any example hereof or a fragment of a gene construct comprising a Ch1 d synthase gene, e.g., an algal chloroplast or plant chloroplast. Optionally, the chloroplast further comprises a gene construct comprising the A. marina CBPII-encoding gene pcbA, In this respect, the gene construct or fragment thereof comprising a Ch1 d synthase gene with any optional A. marina pcbA gene, may be present as an extrachomosomal genetic element or episome in the chloroplast e.g., as a plasmid or cosmid, or integrated into the chloroplast genome.

In a further example, the present invention extends to any cell, tissue, organ or whole organism comprising a gene construct of the present invention as described according to any example hereof or a fragment of a gene construct comprising a Ch1 d synthase gene with any optional A. marina pcbA gene. Preferred tissues, organs or whole organisms are plant tissues, plant organs, and whole plants.

Preferred chloroplasts, cells, tissues, organs and whole organisms according to the present invention have or express a modified phenotype or trait or a new phenotype or trait by virtue of the introduced Ch1 d synthase gene or expression of the introduced Ch1 d synthase gene, and any optional co-expressed A. marina pcbA gene, e.g., as described herein above.

In another example, the present invention provides a method for producing a transgenic chloroplast or cell, said method comprising introducing a gene construct of the present invention or a fragment of a gene construct comprising a Ch1 d synthase gene into a chloroplast or cell, optionally in combination with A. marina pcbA gene. Suitable methods for introducing a nucleic acid into a cell will be apparent to the skilled artisan, e.g., transformation using CaCl₂ and variations thereof, PEG-mediated uptake, microparticle bombardment, electroporation, microinjection, vacuum-infiltration of tissue or Agrobacterium-mediated transformation. Preferably, the method additionally comprises producing, providing or obtaining the gene construct(s) or fragment(s) thereof.

This method may produce transgenic plant tissue or a transgenic plant. In one example, the method may comprise contacting a transgenic plant cell with a compound that induces callus formation and/or induces dedifferentiation of the transgenic cell (or a cell derived therefrom) and/or induces the production of an undifferentiated cell from said transgenic cell for a time and under conditions sufficient to produce a callus and/or dedifferentiated cell and/or undifferentiated cell. By “callus” is meant a cluster or group of undifferentiated cells resulting from cell division in the absence of regeneration. By “regeneration” is meant a process by which a plant or plant part e.g., leaf tissue, especially a plantlet, is produced from a transgenic plant cell e.g., by a process of organogenesis or embryogenesis. As used herein, the term “organogenesis” shall be taken to mean a process by which shoots and roots are developed sequentially from meristem centres. As used herein, the term “embryogenesis” shall be taken to mean a process by which shoots and roots develop together in a concerted fashion (not sequentially), whether from somatic cells or gametes. Those skilled in the art are aware that a transgenic plant cell can be used without undue experiment to produce a transgenic plant, e.g., by regeneration.

In a further example, the present invention also provides for use of a transgenic plant cell comprising a Ch1 d synthase gene of the present invention and any optional co-expressible A. marina pcbA gene, in the production of a transgenic plant or plantlet. As used herein, the term “plantlet” shall be taken to mean a shoot or root that has developed from a plant cell, e.g., using in vitro techniques. For example, a plantlet is a shoot or root that has been induced to grow from a callus using a compound, such as, for example, indole-3-acetic acid, benzyladenine, indole-butyric acid, zeatin, α-naphthaleneacetic acid, 6-benzyl aminopurine, thidiazuron or kinetin, 2iP. The term “plant” refers to an entire plant comprising hypocotyl and epitcotyl, or shoot and root structures.

In yet, another example, the present invention provides a method for producing a transgenic plant or plantlet, said process comprising:
(i) providing, producing or obtaining a transgenic plant cell or callus comprising a gene construct of the present invention or a fragment of a gene construct comprising a Ch1 d synthase gene and any optional co-expressible A. marina pcbA gene; and
(ii) regenerating a transgenic plant or plantlet from the transgenic plant cell or callus at (i), thereby producing a transgenic plant or plantlet.

In an alternative example, the present invention provides a method for producing a transgenic plant or plantlet, said process comprising:
(i) providing, producing or obtaining a transgenic chloroplast comprising a gene construct of the present invention or a fragment of a gene construct comprising a Ch1 d synthase gene and any optional co-expressible A. marina pcbA gene; and
(ii) providing, producing or obtaining a transgenic plant cell or callus comprising the transgenic chloroplast at (i); and
(iii) regenerating a transgenic plant or plantlet from a cell or callus at (ii), thereby producing a transgenic plant or plantlet.

Methods for regenerating a plant or plantlet from a plant cell or callus will be apparent to the skilled artisan and/or described herein. For example, a transgenic plant cell is contacted with a compound that induces callus formation and/or induces dedifferentiation of the transgenic cell (or a cell derived therefrom) and/or induces the production of an undifferentiated cell from said transgenic cell for a time and under conditions sufficient to produce a callus and/or dedifferentiated cell and/or undifferentiated cell, e.g., a compound described supra. Callus is generally contacted with a compound that induces shoot and/or root formation, e.g., a compound described supra for the production of a plantlet for a time and under conditions for a plantlet to form. To produce a whole plant a plantlet is grown for a time and under conditions for it to develop into a whole plant (e.g., grow to maturity).

This method extends to providing or obtaining from the transgenic plant or plantlet, an offspring plant and/or seed and/or propagating material and/or reproductive material and/or germplasm and/or vegetative material comprising the gene construct of the present invention or a fragment of a gene construct comprising a Ch1 d synthase gene and any optional co-expressible A. marina pcbA gene.

This method also extends to growing or maintaining or breeding the transgenic plant or plantlet for a time and under conditions sufficient for seed to be produced, and optionally, to obtaining seed comprising the introduced gene construct or a fragment thereof comprising a Ch1 d synthase gene and any optional co-expressible A. marina pcbA gene. The term “breeding” is to be taken in its broadest context to mean any process by which a zygote and/or an offspring plantlet or plant is produced from or using a parent plant a part thereof or a cell thereof. For example, the term “breeding” encompasses sexual reproduction such as, cross-breeding or cross-pollination, whereby reproductive material, e.g., pollen from one plant is used to fertilize reproductive material, e.g., an egg cell within an ovule from another plant. The term “breeding” also encompasses sexual reproduction such as selling or self-fertilization, whereby reproductive material from a plant, e.g., pollen is used to fertilize reproductive material, e.g., an egg cell within an ovule, from the same plant. The term “breeding” also encompasses vegetative forms of reproduction, such as the production of a plant from a stolon or a rhizome or a bulb or a tuber or a corm or a cutting or a graft or a bud. The term “breeding” also encompasses in vitro methods, e.g., in vitro fertilization and zygote culture.

In the case of sexual reproduction, the present invention provides a method for breeding a transgenic plant, said method comprising:
(i) providing, producing or obtaining a transgenic plant comprising a gene construct of the present invention or a fragment of a gene construct comprising a Ch1 d synthase gene and any optional co-expressible A. marina pcbA gene; and
(ii) breeding the transgenic plant produced at (i) to thereby produce a zygote comprising the gene construct or fragment thereof comprising a Ch1 d synthase gene and optional co-expressible A. marina pcbA gene.

Alternatively, the method comprises:
(i) providing, producing or obtaining plant reproductive material comprising a gene construct of the present invention or a fragment of a gene construct comprising a Ch1 d synthase gene and any optional co-expressible A. marina pcbA gene; and
(ii) combining reproductive material of a plant with the reproductive material at (i) such that a zygote comprising the gene construct or fragment is produced.

Preferably, the method additionally comprises growing the zygote to form a transgenic plant. In one example, the step of obtaining a transgenic plant comprises obtaining a seed or a plantlet or a pant part comprising a promoter, active fragment, derivative, expression construct or expression vector of the present invention, and growing said seed plantlet or plant or plant part e.g., leaf tissue to thereby obtain the transgenic plant.

In the case of cross-breeding, the transgenic plant is bred with or transgenic reproductive material is combined with a transgenic plant or transgenic reproductive material to produce a zygote, plant, plantlet or plant part e.g., leaf tissue homozygous or heterozygous for a promoter, active fragment, derivative, expression construct or expression vector of the present invention. Alternatively, the transgenic plant is bred with or transgenic reproductive material is combined with a wild-type plant or wild-type reproductive material to produce a zygote, plant, plantlet or plant part e.g., leaf tissue heterozygous for the Ch1 d synthase gene.

Preferably, a method of breeding of the present invention additionally comprises selecting or identifying a zygote, plantlet, plant part or whole plant comprising the Ch1 d synthase gene. When the co-expressible A. marina pcbA gene has also been used, it is preferable to select for zygotes that comprise both introduced genes i.e., Ch1 d synthase and pcbA.

In one example, a method of breeding of the present invention additionally comprises detecting expression or a pattern of expression of the Ch1 d synthase gene in a plantlet, plant part or whole plant, and optionally also detecting expression of CBPII when a co-expressible A. marina pcbA gene has also been used to produce the plantlet, plant part or whole plant.

In the case of vegetative reproduction, the present invention provides a method comprising:
(i) providing, producing or obtaining a transgenic plant, plantlet or plant part e.g., leaf tissue comprising a gene construct or fragment thereof comprising a Ch1 d synthase gene and an optional co-expressible A. marina pcbA gene; and
(ii) maintaining the transgenic plant, plantlet or plant part for a time and under conditions sufficient for the plant to reproduce vegetatively.

Suitable conditions will depend on the form of vegetative reproduction and will be apparent to the skilled artisan. For example, a lateral shoot from a plant is induced to form adventitious roots by burying the shoot and, following adventitious root formation, the shoot is separated from the parent plant and a new plant grown. Alternatively, or in addition, a plant or plantlet or plant part is induced to form a callus, e.g., by cutting a part of the plant, plant part or plantlet or using a process described supra, and the callus maintained under conditions sufficient to a plantlet or plant to grow.

Preferably, a method supra further comprises determining expression or a pattern of expression of an introduced Ch1 d synthase gene, and optionally, determining expression or a pattern of expression of a co-expressible A. marina pcbA gene, in a plant, plant cell or plant part.

Plants, algae and cyanobacteria expressing the Ch1 d synthase-encoding nucleic acid of the invention such produce Ch1 d in plastids or cells de novo, or produce an enhanced level of Ch1 d relative to otherwise isogenic counterparts that do not express the introduced Ch1 d synthase gene. This is also true if such plants, algae and cyanobacteria co-express an optional A. marina pcbA gene. By virtue of such expression, the transformed organisms of the invention are less susceptible to environmental factors such as light intensity and/or light quality, and/or have an ability to harvest light of different quality, or grow more rapidly, compared to their naturally-occurring counterparts. Such organisms have increased yield characteristics and/or greater environmental range than their non-transgenic counterparts. In one example, organisms engineered to produce Ch1 d in a background that does not normally produce the pigment, such as plants and Ch1 a/b algae, grow more efficiently in lower light intensities than otherwise isogenic non-transformed counterparts. Alternatively, or in addition, such organisms grow more efficiently under near-infrared light e.g., with less orange-red light and/or less blue light, such as under incandescent light. Alternatively, or in addition, such organisms grow more efficiently in short day photoperiods than otherwise isogenic non-transformed counterparts. Alternatively, or in addition, such organisms grow more efficiently in early morning and/or late afternoon than otherwise isogenic non-transformed counterparts. Alternatively, or in addition, such organisms grow more efficiently during late summer and/or autumn/fall and/or during winter than otherwise isogenic non-transformed counterparts. As used herein, such efficient growth comprises any increase in biomass e.g., as a consequence of increased photosynthetic capacity and/or carbon fixation ability. A more efficient growth may therefore manifest as one or more yield traits e.g., enhanced cell expansion rate, enhanced cell size, enhanced cell division, enhanced vegetative growth or leaf size or leaf area or seed number or seed size. Other yield traits enhanced by increasing photosynthetic capacity and/or carbon fixation ability will be known to those skilled in the art. In another example, cyanobacteria that normally produce Ch1 d and are engineered to ectopically-express the Ch1 d synthase-encoding nucleic acid of the invention are faster-growing e.g., they have shorter doubling times, than their non-transgenic counterparts, Clearly, such transformed organisms have enhanced utility as a source of feed, feedstock, and secondary metabolites e.g., biofuels, oils, nutraceuticals, specialty chemicals, pharmaceuticals, etc.

It will also be apparent from the preceding description that the Ch1 d synthase-encoding nucleic acid of the invention provides a means for producing produce Ch1 d in organisms that do not normally produce the pigment. By virtue of the fluorescence of Ch1 d at 710 nm, distinct from the emission spectra for other chlorophylls, the invention provides for a use of the subject nucleic acid to identify and track plants and algae, and cells and chloroplasts derived from such plants and algae e.g., in the field or in culture, by virtue of a conferred ability to produce Ch1 d. Conveniently, plants or algae into which the nucleic acid of the invention is introduced at least produce Ch1 a.

In another example, the Ch1 d synthase-encoding nucleic acid of the invention provides a means for producing produce Ch1 d in organisms that do not normally produce any chlorophyll pigment, wherein the subject nucleic acid is expressed in the presence of exogenous Ch1 a substrate e.g., under low light conditions or in darkness and/or under aerobic growth conditions and/or under a low concentration of carbon monoxide such as occurs in the atmosphere. Optionally, the Ch1 d synthase-encoding nucleic acid of the invention is co-expressed with one or more other elements of the photosynthetic machinery in such organisms, e.g., the A. marina CBPII-encoding gene, pcbA.

In another example, the present invention provides an antibody that binds to Ch1 d synthase polypeptide or a fragment thereof such as a B-cell epitope. For example, the antibodies bind to cyanobacterial Ch1 d synthase polypeptide, and more particularly to a Ch1 d synthase from an isolate of A. marina described according to any example hereof. The exemplified antibodies are polyclonal antibodies produced against a multi-epitope immunogen derived from the full-length amino acid sequence of the encoded protein, however a skilled immunologist would have no difficulty in producing monoclonal antibodies based on the amino acid sequence data provided for full-length Ch1 d synthase polypeptide, and/or the sequence of the multi-epitope immunogen hereof. For example, a suitable immunogen for the production of monoclonal and/or polyclonal antibodies may comprise amino acid residues 20-29 of SEQ ID NO: 12 and/or amino acid residues 127-136 of SEQ ID NO: 12 and/or amino acid residues 260-269 of SEQ ID NO: 12 and/or amino acid residues 336-365 of SEQ ID NO: 12 and/or amino acid residues 426-435 of SEQ ID NO: 12 and/or amino acid residues 501-510 of SEQ ID NO: 12. Other immunogenic epitopes of a full-length or partial Ch1 d synthase polypeptide comprising a sequence having at least 70% or 75% or 80% or 85% identity to SEQ ID NO: 12 may be utilized for antibody production without undue experimentation. In a particularly preferred example, an immunogenic polypeptide for production of antibody that binds to Ch1 d synthase polypeptide comprises the amino acid sequence of SEQ ID NO: 37 hereof or at least about 10 or 20 or 30 or 40 or 50 contiguous residues thereof.

### 2. General

This specification contains nucleotide and amino acid sequence information prepared using PatentIn Version 3.5 presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence are indicated by information provided in the numeric indicator fields <211>, <212> and <213>, respectively. Nucleotide sequences referred to in the specification are defined by the term “SEQ ID NO:”, followed by the sequence identifier (e.g. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and/or all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

As used herein the term “derived from” shall be taken to indicate that a specified integer may be obtained from a particular source albeit not necessarily directly from that source.

The following prior disclosures provide conventional techniques of molecular biology, microbiology, and recombinant DNA technology:
I. Sambrook, Fritsch & Maniatis, Molecular Cloning: A Laboratory Manual, Cold Spring Harbor Laboratories, New York, Third Edition (2001), whole of Vols I, II, and III;
2. DNA Cloning: A Practical Approach, Vols. I and II (D. N, Glover, ed., 1985), IRL. Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds 1985) IRL Press, Oxford, whole of text;
5. Immobilized Cells and Enzymes: A Practical Approach (1986) IRL Press, Oxford, whole of text;
6. Perbal, B., A Practical Guide to Molecular Cloning (1984);
7. Methods In Enzymology (S. Colowick and N. Kaplan, eds., Academic Press, Inc.), whole of series;
8. McPherson et al., In: PCR A Practical Approach., IRL Press, Oxford University Press, Oxford, United Kingdom, 1991.

### BRIEF DESCRIPTION OF THE DRAWINGS

FIG. 1 provides the chemical structures of Ch1 a and Ch1 d and their absorbance spectra (in methanol.

FIG. 2 provides mass spectra of (A) Ch1 a and (B) Ch1 d extracted from Acaryochloris marina cells that have been cultured in medium comprising H₂¹⁸O (labelled water) for 48 hours.

FIG. 3a provides a graphical time course of the relative incorporation rate (%) of ¹⁸O from labeled water into Ch1 a. The filled diamonds represent the percentage of Ch1 a without ¹⁸O incorporated i.e., ¹⁸O₀ having a mass of unlabeled Ch1 a standard mass. The squares with crosses represent percentages of Ch1 a molecules having one to four ¹⁸O atoms (¹⁸O_{1.4}). The filled circles represent the percentage of Ch1 a molecules having five ¹⁸O atoms i.e., ¹⁸O₅.

FIG. 3b provides a graphical time course of the relative incorporation rate (%) of ¹⁸O from labeled water into Ch1 d. The filled diamonds represent the percentage of Ch1 d without ¹⁸O incorporated i.e., ¹⁸O₀ having a mass of unlabeled Ch1 d standard mass. The squares with crosses represent percentages of Ch1 d molecules having one to four ¹⁸O atoms (¹⁸O_{1.4}). The filled circles represent the percentage of Ch1 d molecules having five ¹⁸O atoms i.e., ¹⁸O₅.

FIG. 4a provides a graphical time course of the relative incorporation rate (%) of ¹⁸O from labeled oxygen gas i.e., ¹⁸O₂ into Ch1 a. The filled diamonds represent the percentage of Ch1 a without ¹⁸O incorporated i.e., ¹⁸O₀ having a mass of unlabeled Ch1 a standard mass. The squares with crosses represent percentages of Ch1 a molecules having one ¹⁸O atom (¹⁸O₁). The open circles represent the percentage of Ch1 a molecules having two ¹⁸O atoms i.e., ¹⁸O₂. The filled circles represent the percentage of Ch1 a, molecules having three or four ¹⁸O atoms i.e., ¹⁸O₃₋₄.

FIG. 4b provides a graphical time course of the relative incorporation rate (%) of ¹⁸O from labeled oxygen gas O₂ into Ch1 d. The filled diamonds represent the percentage of Ch1 d without ¹⁸O incorporated i.e., ¹⁸O₀ having a mass of unlabeled Ch1 d standard mass. The squares with crosses represent percentages of Ch1 d molecules having one ¹⁸O atom (¹⁸O₁). The open circles represent the percentage of Ch1 d molecules having two ¹⁸O atoms i.e., ¹⁸O₂. The filled circles represent the percentage of Ch1 d molecules having three or four ¹⁸O atoms i.e., ¹⁸O_{3.4}.

FIG. 5 is a graphical representation showing changes in the ratio of Ch1 a to Ch1 d in A. marina cells grown under a mixture of air and carbon monoxide (arrow marked “CO+Air”), and then switched to air only (arrow marked “Air”). Diamonds indicate the ratio of Ch1 a to Ch1 d in cells grown under each condition, and the dotted line indicates the relationship of time and changes of ratio of Ch1 a/Ch1 d under CO+Air.

FIG. 6 is a graphical representation showing the changing profile of total chlorophyll (triangles) and ratio of Ch1 a to Ch1 d (squares) in A. marina cells grown under continuous stream of pure nitrogen gas (N₂) for up to 46 hr, then switched to air (Air) for a further 24 hr, followed by a mixture of air and carbon monoxide (CO+Air) for 12 hr, and finally, air (Air) for a further 24 hr. Air was fresh air; CO+Air was 50% CO and 50% fresh air.

FIG. 7 is a graphical representation showing relative mRNA levels compared to 16S rRNA, transcribed from A. marina cytochrome P450 oxygenase-encoding genes AMI_{—}0606, AMI_{—}0824, AMI_{—}3563, AMI_{—}4161, and AMI_{—}5780. A. marina had been cultured under different gases as follows: Air_{—}11, fresh air for 11 hr; Air_{—}23, fresh air for 23 hr; CO_{—}10 Hr, a mixture of fresh air and carbon monoxide for 10 hr, after 23 hr under fresh air; CO_{—}25 Hr, a mixture of fresh air and carbon monoxide for 25 hr, after 23 hr under fresh air; following 23 hr under fresh air; Air_{—}9 Hr, fresh air for 9 hr after 25 hr under a mixture of fresh air and carbon monoxide for 25 hr.

FIG. 8 is a representation showing a SDS/polyacrylamide gel comprising proteins expressed in E. coli transformed with gene constructs comprising the A. cytochrome P450 oxygenase-encoding gene AMI_{—}3563, Lane 1, MW marker; Lane 2, whole cell lysates of cells in which expression was not induced; Lane 3, whole cell lysates of cells in which expression was induced for 3 or 4 hr; Lane 4, soluble cell fraction of whole cell lysates from cells in which expression was induced; Lane 5, membrane fraction of whole cell lysates from cells in which expression was induced; Lane 6, membrane “halo” fraction (inclusion body) from cells in which expression was induced. The circle indicates the position of AMI_{—}3563 protein in the membranes of cells (MW=58 kDa). The arrow indicates a possible degradation product of AMI_{—}3563 sequestered to inclusion bodies.

FIG. 9 provides a graphical representation of fluorescence emission spectra for E. coli incubated with Ch1 a and expressing one of the A. marina cytochrome P450 oxygenase-encoding genes AMI_{—}3563 and AMI_{—}4161, and for three Rieske Fe—S centre domains, AMI_{—}1961, AMI_{—}2850 and AMI_{—}4158, Emission fluorescence was recorded from 650 nm to 750 nm by exciting at 440 nm (Ch1 a). The arrow indicates the presence of a red-shifted peak at 710 nm, corresponding to Ch1 d, for cultures expressing AMI_{—}3563.

FIG. 10 provides a schematic representation of vector pDONR221 and a process for introducing thereto an A. marina Ch1 d synthase-encoding gene such as set forth in any one of SEQ ID Nos: 11, 21, 23, 25 or 27 or amplified using primers set forth in any one of SEQ ID Nos: 18-20, 29-31 or 33-36 as described herein. The A. marina sequence is obtained, preferably using primers comprising 5′-terminal and 3′-terminal AttB1 and AttB2 sites as shown (CH1 d synthase; upper left) for recombination into the acceptor sites attP1 and attP2 of the vector pDONR221 (upper right), and a unique restriction site such as XhoI as indicated to provide for subsequent insertion of a chloroplast transit peptide-encoding sequence such as from the small subunit of rubisco (prSSU) upstream of the open reading frame of A. marina Ch1 d synthase-encoding gene. The plasmid shown at the lower left of the figure is the resultant recombinant plasmid comprising the A. marina Ch1 d synthase-encoding gene (AMI 3563) positioned between recombined sites attL1 and attL2 of the construct. The position of a unique XhoI site in the resultant plasmid is indicated at the 5′-end of the A. marina CH1 d synthase-encoding gene. The positions of kanamycin-resistant gene Kan(R) gene and carbenicillin-resistance gene Cm(R) are also indicated.

FIG. 11 provides a schematic representation of a process for constructing a plant transformation binary vector comprising the A. marina Ch1 d synthase-encoding gene such as set forth in any one of SEQ ID Nos: 11, 21, 23, 25 or 27 or amplified using primers set forth in any one of SEQ ID Nos: 18-20, 29-31 or 33-36 as described herein. The A. marina Ch1 d synthase-encoding gene of the construct produced as described in the legend to FIG. 10 (upper left) is sub-cloned by recombination between the attL1/attL2 sites of that construct with attR1/attR2 sites of the vector pK7WG2 (upper right), thereby replacing the CmR-ccdB gene of pK7WG2 with the Ch1 d synthase-encoding gene positioned downstream and operably under control of the CaMV 35S promoter (p35S), and upstream of the CaMV 35S transcription termination signal (T35S). Plasmid pK7WG2 also comprises a sequence encoding green fluorescent protein (GFP) upstream of the CaMV 35S transcription termination signal (T35S) for use in detecting transformed cells (not shown). The resultant binary plasmid pK7FWG2-AMI 3563 (lower left) thus comprises the A. marina Ch1 d synthase-encoding gene and downstream GFP tag (Egfp) operably under control of CaMV 35S promoter and with a unique XhoI site at the 5′-end of the A. marina Ch1 d synthase-encoding open reading frame (circled) for subsequent insertion of a chloroplast transit peptide-encoding sequence such as from the small subunit of rubisco (prSSU). The positions of the A. tumefaciens left border (LB) and right border (RB) sequences for integration into plant genomic DNA are indicated in both pK7FWG2 and pK7FWG2-AMI 3563. The position of a plant-operable kanamycin-resistance gene (Kan) nearer the LB sequence is also indicated.

FIG. 12 provides a schematic representation of the binary plasmid vector pt-gk (left) comprising the Tin enhancer operably linked to a sequence encoding the 180-amino acid small subunit of rubisco (prSSU), as a source of nucleic acid (right) comprising a sequence encoding chloroplast transit peptide. In plasmid pt-gk, the prSSU sequence and optional Tin enhancer are flanked by sequences to facilitate sub-cloning at the 5′-end of the A. marina Ch1 d synthase-encoding open reading frame in plasmid pK7FWG2-AMI 3563 (FIG. 11). The Tin-prSSU sequence of pt-gk may be obtained with the required flanking sites for insertion into pK7FWG2-AMI 3563 by amplification using primers comprising a suitable restriction enzyme target site e.g., XhoI. The Tin enhancer sequence is optional in this construct. The prSSU sequence may also be derived from any higher plant e.g., tobacco such as N. tabacum or N. benthamiana.

FIG. 13 provides a schematic representation of the binary plasmid vector pK7FWG2-AMI 3563(XhoI) for plant transformation. This vector comprises the A. marina Ch1 d synthase-encoding gene fused to an upstream sequence encoding the prSSU (including chloroplast transit peptide-encoding sequence) and downstream sequence encoding a GFP tag (Egfp), wherein the fusion polypeptide is operably under control of CaMV 35S promoter. The prSSU sequence has been introduced at the unique XhoI site at the 5′-end of the A. marina Ch1 d synthase-encoding open reading frame of plasmid pK7FWG2-AMI 3563 (FIG. 11). The positions of the A. tumefaciens left border (LB) and right border (RB) sequences for integration into plant genomic DNA are also indicated. The position of a plant-operable kanamycin-resistance gene (Kan) nearer the LB sequence is also indicated.

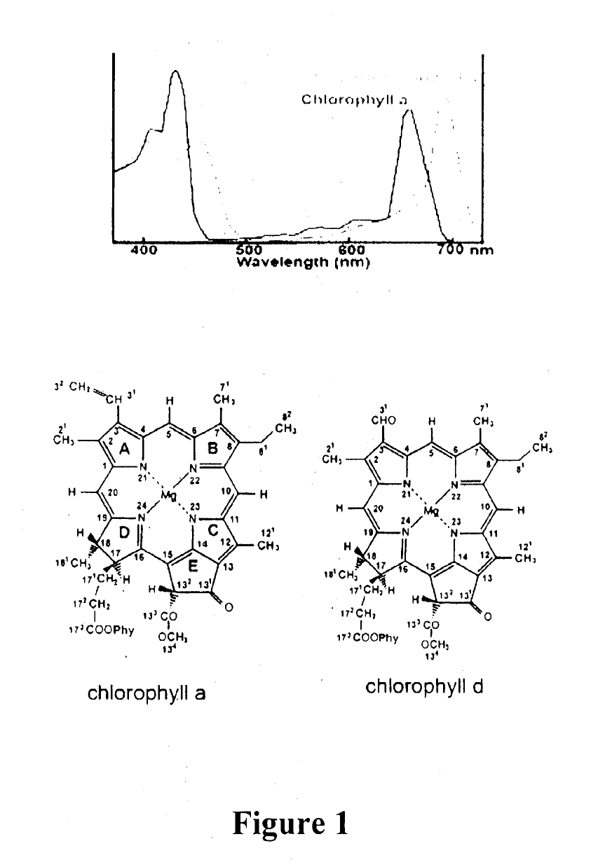

### DESCRIPTION OF THE PREFERRED EMBODIMENTS

Variants of the Exemplified A. marina Ch1 d Synthase Gene and Polypeptide

To produce variants of SEQ ID NO: 11 or a variant thereof described herein e.g., any one of SEQ ID Nos: 21, 23, 25 or 27, one or more nucleotide residues of the exemplified sequence is modified by art-recognized methods e.g., by substitution of one or more nucleotides in one or more codons of SEQ ID NO: 11 or the variant to thereby produce nucleic acid comprising a sequence that is degenerate with SEQ ID NO: 11 or the variant by virtue of the genetic code and/or that varies from SEQ ID NO: 11 or the variant by virtue of a codon usage bias. In so doing, the structure of the encoded protein will be substantially identical or identical to the structure of the native A. marina Ch1 d synthase of the present invention. This means that the primary amino acid sequence of the encoded Ch1 d synthase variants in such circumstances will be substantially identical or identical to the sequence of the A. marina Ch1 d synthase polypeptide set forth herein as SEQ ID NO: 12 or a variant thereof described herein e.g., any one of SEQ ID Nos: 22, 24, 26 or 28. Accordingly, such variants are structurally and functionally homologous to the “parent” A. marina Ch1 d synthase gene from which they are derived by the hand of man or to which they are related in nature.

For example, the isolated A. marina Ch1 d synthase gene exemplified herein is modified by any available mutagenesis method to include one or more variant codons that encode the same amino acid residue based on the genetic code. When expressed, this mutagenized nucleic acid encodes a Ch1 d synthase polypeptide that is structurally and functionally the same as the A. marina polypeptide of SEQ ID NO: 12 or the variant e.g., any one of SEQ ID Nos: 22, 24, 26 or 28.

For example, site-directed mutagenesis is of general applicability for producing variants of the Ch1 d synthase gene of the present invention comprising one or more nucleotide substitutions at one or more predetermined sites in SEQ ID NO: 11 or a variant thereof. See, e.g., Botstein et al., Science 229, 1193-1201 (1985); Itakura et al., Ann. Rev. Biochem. 53, 323-356 (1984); Smith Ann. Rev. Genet. 19, 423-462 (1985). The present invention clearly encompasses the simultaneous production of a library of variants e.g., wherein each comprises a different substitution at the same nucleotide position or at several positions, such as by saturation mutagenesis e.g., McGrath et al., Nature 310, 644-649 (1984); Seeburg et al., Nature 312, 71-75 (1984); Bargmann et al., EMBO J. 7, 2043-2052 (1988).

In one example, site-directed mutagenesis and/or saturation mutagenesis is performed using one or more oligonucleotides comprising the mutated codon sequence i.e., in so-called oligonucleotide-directed mutagenesis e.g., Itakura et al., Ann. Rev. Biochem. 53, 323-356 (1984); Smith Ann. Rev. Genet. 19, 423-462 (1985); Zoller et al., Nucl. Acids Res. 10, 6487-6500 (1982). In this approach, a template molecule is prepared by cloning the A. marina Ch1 d synthase gene or a fragment thereof into a phage M 13-based single-stranded cloning vector, and mutagenesis is performed by hybridizing to the vector a mutagenic oligonucleotide primer comprising one or more mutated codons and having sufficient complementarity for hybridization to occur over at least a portion of the primer comprising a 3′-end thereof, synthesizing nucleic acid to thereby incorporate the mutagenic oligonucleotide primer, and ligating the synthesized nucleic acid incorporating the mutagenic oligonucleotide primer. Cells e.g., bacterial cells, are then transformed with the ligated nucleic acid, and transformed cells are identified e.g., by standard hybridization employing the mutagenic oligonucleotide primer and/or nucleotide sequence analysis. By using a panel of mutagenic oligonucleotides comprising different substitutions at the targeted codon(s), a position can be saturated with mutations. Specific mutagenic primers may be employed for each specific codon modification, or a pool of degenerate oligonucleotide primers may be employed encompassing all possible mutations to be introduced at each targeted region of the gene.

In another example, a PCR-based approach is employed to introduce specific mutations into DNA may also be employed utilizing specific mutagenic oligonucleotide primers for each mutation e.g., as described by Higuchi et al., Nucl. Acids Res. 16, 7351-7367 (1988) or Ho et al., Gene 77, 51-59 (1989).

In another example, a form of cassette mutagenesis is employed e.g., where a targeted region is contained on a small DNA fragment flanked by unique restriction endonuclease cleavage sites i.e., sites that do not occur elsewhere in the target plasmid e.g., as described by Matteucci et al., Nucl. Acids Res. 11, 3113-3121 (1983); Wells et al., Gene 34, 315-323 (1985); Derbyshire et al., Gene 46, 145-152 (1986). In cassette mutagenesis, cleavage with restriction enzymes excises a small segment of DNA, which is then replaced with a single-stranded or double-stranded DNA segment comprising the mutated sequence. The requirement for nearby unique restriction sites (or sequences that can be converted into unique sites by mutagenesis) is overcome by the use of a BspMI cassette comprising the DNA to be mutagenized flanked by two BspMI recognition sites arranged in opposite orientations, wherein the BspMI cassette is digested with BspMI to thereby remove the DNA to be mutagenized and leave an acceptor molecule, and a mutagenic oligonucleotide is inserted into the acceptor molecule to restore the open reading frame albeit comprising the mutated sequence, see e.g., Stone et al., Mol. Cell. Biol. 8, 3565-3569 (1988).

In another example, codon cassette mutagenesis is employed e.g., as described by Kegler-Ebo et al., Nucl. Acids Res. 22, 1593-1599 (1994). In this approach, a set of universal mutagenic cassettes is employed to deposit mutant codons into the A. marina Ch1 d synthase gene. A requirement for using codon cassette mutagenesis is that the starting target plasmid contains no endogenous SapI cleavage sites, which are readily removed by point mutation without altering the amino acids encoded by the open reading frame. An advantage of codon cassette mutagenesis is that the same set of mutagenic codon cassettes can be used for codon replacement with all possible target molecules. Thus, once target molecules are constructed and the set of mutagenic codon cassettes is obtained, all possible single codon substitution mutations can be generated with no additional expense for oligonucleotide synthesis. The position of the blunt-ends in the target molecule is manipulated to ensure that the mutation is a codon replacement. The only mutations that must be custom-designed and constructed in codon cassette mutagenesis are those that allow the generation of the unique blunt-ends at the site of the codon replacement, and standard cassette, PCR-based or M13-based mutagenesis procedures are employed to achieve that purpose. Conveniently, restriction endonucleases with blunt-end cleavage sites are used to generate blunt-ends at the appropriate positions. This is not problematic because, for example, the alanine codon GCC, the arginine codon AGG, the asparagine codon AAU, the aspartic acid codon GAU, the cysteine codon UGC, the glutamic acid codon GAG, the glutamine codon CAG, the glycine codon GGC, the histidine codon CAC, the phenylalanine codon UUU, the proline codons CCC and CCG, the serine codons UCG and AGC and AGU, the tryptophan codon UGG, the tyrosine codon UAC, and the valine codons GUU and GUA and GUC each immediately precede a blunt-end restriction endonuclease cleavage site; and in the opposing DNA strand the alanine codons GCC and GCA and GCU, the arginine codons CGA and CGG and CCG, the asparagine codon AAC, the aspartic acid codon GAC, the glycine codons GGC and GCC and GGG, the isoleucine codons AUC and AUU, the leucine codons CUG and CUC and CUC, the lysine codon AAA, the proline codons CCA and CCU, the threonine codon ACU, the tyrosine codon UAC and the valine codons GUA and GUG each immediately follow a blunt-end restriction endonuclease cleavage site. On the other hand, not all amino acid codons are accommodated by available blunt-end restriction endonucleases.

In another example, a chemical or enzymatic method is employed to subject a short sequence of the Ch1 d synthase gene to intense mutagenesis e.g. Myers et al., Science 229, 242-246 1985).

Depending upon the organism in which Ch1 d synthase of the invention is to be expressed, some optimization of codon usage may be employed to thereby provide for a higher level of expression and/or rate of mRNA translation than the native A. marina mRNA sequence in the target organism. However, it is to be understood that such modification are not essential to performance because functionality may not be affected adversely by amino acid substitutions or read-through the standard termination codon of SEQ ID NO 11 or a variant thereof. There is mounting evidence that limited amino acid replacements most often have a minimal effect on the structure and performance of proteins and bacteria in particular may tolerate sub-optimal codon usage e.g., Dean et al., Mol. Biol. Evol. 5, 469-485 (1988); Kurland et al. In: Gene expression: the translational step and its control (B. F. C. Clark and H. U. Petersen, eds), pp 193-203 Munksgaard, Copenhagen. (1984). Should such substitution be desired, any one or more of the methods described herein above may be employed for this purpose based on a known codon usage bias or codon usage preference for the target organism. As with any degenerate sequence, the structure of the encoded protein is substantially the same as that of the A. marina Ch1 d synthase protein, e.g., with the possible exceptions of translation start and termination codons.

Exemplary albeit non-exhaustive codon reassignments may be based on the ability of glutamine tRNAs of Tetrahymena spp. to read the termination codons UAA and UAG, and for certain prokaryotic organisms and organelles such as those having A+T-rich genomes to read the conventional termination signal UGA as a codon for tryptophan and/or the standard isoleucine codon AUA as a codon for methionine.

Alternatively, or in addition, exemplary degenerate nucleotide substitutions are based on a codon usage bias. For example, there appears to be a preferential usage of codons ending in A or U by chloroplast genomes e.g., that are A+T rich, compared to a preferential use of codons ending in G or C by many cyanobacteria and the nuclear genomes of green algae, and slight preferences of dicotyledonous plants for codons ending in A or U, and of monocotyledonous plants for codons ending in C or G or similar to the nuclear genes of green algae. Nakamura et al., Nucleic Acids Res. 28, p292 et seq. (2000) disclose codon usage frequencies for 257,468 complete protein-encoding sequences (CDSs) from 8792 organisms, also obtainable from the website of Kazusa, as a source of codon usage tables for selected species in determining such modifications to the Ch1 d synthase gene of the present invention.

The mutation process infra for producing variants of the A. marina Ch1 d synthase-encoding gene, may also be employed to replace one or more standard codons in the parent molecule for a different codon encoding a different amino acid, thereby changing one or more standard amino acids in the resulting mutant protein. Similarly, one or more additional amino acids may be inserted at the N-terminus or C-terminus of the molecule, or introduced internally, by such nucleic acid mutational processes. It is also possible to employ such procedures to delete one or more amino acids from the native molecule. The present invention clearly encompasses such mutational approaches provided that the encoded polypeptide retains Ch1 d synthase activity or has enhanced Ch1 d synthase activity relative to A. marina Ch1 d synthase comprising the amino acid sequence of SEQ ID NO: 12 or a variant thereof when assayed in the same cell under the same conditions.

In modifying the amino acid sequence of A. marina Ch1 d synthase set forth in SEQ ID NO: 12 or a variant thereof, it is preferred to substitute one amino acid in the “parent molecule” for another amino acid of similar charge and/or hydrophilicity and/or size, and conservative amino acid substitutions are particularly preferred. To produce a conservative amino acid substitution, a mutational approach as described herein is modified by utilizing a mutagenic oligonucleotide comprising a codon sequence that specifies a different amino acid.

In one example, one or more glycine codons of SEQ ID NO: 11 or a variant thereof i.e., GGU or GGC or GGA or GGG is substituted for an alanine codon e.g., GCU or GCC or GCA or GCG. In another example one or more alanine codons of SEQ ID NO: 11 or a variant thereof i.e., GCU or GCC or GCA or GCG is substituted for a glycine codon e.g., GGU or GGC or GGA or GGG. In another example, one or more aspartate codons of SEQ ID NO: 11 or a variant thereof i.e., GAU or GAC is substituted for a glutamate codon e.g., GAA or GAG. In another example one or more glutamate codons of SEQ ID NO: 11 or a variant thereof i.e., GAA or GAG is substituted for an aspartate codon e.g., GAU or GAC. In another example, one or more lysine codons of SEQ ID NO: 11 or a variant thereof i.e., AAA or AAG is substituted for an arginine codon e.g., CGU or CGC or CGA or CGG or AGA or AGG. In another example one or more arginine codons of SEQ ID NO: 11 or a variant thereof i.e., CGU or CGC or CGA or CGG or AGA or AGG is substituted for a lysine codon e.g., AAA or AAG. In another example, one or more asparagine codons of SEQ ID NO: 11 or a variant thereof i.e., AAU or AAC is substituted for a glutamine codon e.g., CAA or CAG. In another example one or more glutamine codons of SEQ ID NO: 11 or a variant thereof i.e., CAA or CAG is substituted for an asparagine codon e.g., AAU or AAC. In another example, one or more valine codons of SEQ ID NO: 11 or a variant thereof i.e., GUU or GUC or GUG or GUA is substituted for an isoleucine codon e.g., AUU or AUC or AUA or a leucine codon e.g., UUA or UUG or CUU or CUC or CUA or CUG. In another example one or more isoleucine codons of SEQ ID NO: 11 or a variant thereof i.e., AUU or AUC or AUA is substituted for a valine codon e.g., GUU or GUC or GUG or GUA or a leucine codon e.g., UUA or UUG or CUU or CUC or CUA or CUG. In another example one or more leucine codons of SEQ ID NO: 11 or a variant thereof i.e., UUA or UUG or CUU or CUC or CUA or CUG is substituted for a valine codon e.g., GUU or GUC or GUG or GUA or an isoleucine codon e.g., AUU or AUC or AUA. In another example, one or more phenylalanine codons of SEQ ID NO: 11 or a variant thereof i.e., UUU or UUC is substituted for a tryptophan codon UGG or a tyrosine codon e.g., UAU or UAC. In another example one or more tryptophan codons of SEQ ID NO: 11 or a variant thereof i.e., UGG is substituted for a phenylalanine codon e.g., UUU or UUC or a tyrosine codon e.g., UAU or UAC. In another example one or more tyrosine codons of SEQ ID NO: 11 or a variant thereof i.e., UAU or UAC is substituted for a phenylalanine codon e.g., UUU or UUC or a tryptophan codon UGG. In making these substitutions it is to be understood that the designations of uracil (U) in a codon is equivalent to a designation of thymidine (T) in the open reading frame of SEQ ID NO: 11 or the variant. The present invention clearly encompasses any and all variants of SEQ ID NO: 11 encoding a functional Ch1 d synthase polypeptide that comprises combinations of such conservative amino acid substitutions. The present invention also encompasses any and all variants of SEQ ID NO: 12 or a variant thereof described herein that are functional Ch1 d synthase polypeptides comprising combinations of such conservative amino acid substitutions.

In yet another example, a variant of SEQ ID NO: 11 or other exemplified variant Ch1 d synthase gene comprises a nucleotide sequence produced by a nucleic acid sequence-based amplification (NASBA) employing one or more primer sequences comprising SEQ ID NO: 14 and SEQ ID NO: 15 or SEQ ID NO: 18 and SEQ ID NO: 20 or SEQ ID NO: 19 and SEQ ID NO: 20. It is preferred for the purposes of obtaining the full-length open reading frame of a Ch1 d synthase-encoding gene of the invention to employ amplification primers comprising SEQ ID NO: 14 and/or SEQ ID NO: 15 or SEQ ID NO: 18 and SEQ ID NO: 20. For the purposes of nomenclature, these primers correspond to the 5′-end sequence and the complement of the 3′-end sequence of SEQ ID NO: 11. Longer primers than those exemplified by SEQ ID NOs: 14, 15, 18, 19 and 20 may be employed without loss of specificity and, in some circumstances, primers that are extended in the 3′-direction relative to SEQ ID NO: 14 and/or SEQ ID NO: 15 and/or SEQ ID NO: 18 and/or SEQ ID NO: 19 and/or SEQ ID NO: 20 may provide for a greater degree of specificity in amplifying Ch1 d synthase-encoding sequences.

For example, To produce a 3′-end extension of a primer comprising SEQ ID NO: 14, one or more additional nucleotides from position 9 onwards in SEQ ID NO: 11 may be added sequentially to the sequence of SEQ ID NO: 14 and the corresponding oligonucleotide primer synthesized according to standard procedures. For example, 1 or 2 or 3 or 4 or 5 or 6 or 7 or 8 or 9 or 10 or 11 or 12 or 13 or 14 or 15 or 16 or 17 or 18 or 19 or 20 or 21 or more nucleotides from position 9 of SEQ ID NO: 11 may be added to SEQ ID NO: 14. To produce a 3′-end extension of a primer comprising SEQ ID NO: 15, one or more additional nucleotides from position 9 onwards in the complement of SEQ ID NO: 11 may be added sequentially to the sequence of SEQ ID NO: 15 and the corresponding oligonucleotide primer synthesized according to standard procedures. For example, 1 or 2 or 3 or 4 or 5 or 6 or 7 or 8 or 9 or 10 or 11 or 12 or 13 or 14 or 15 or 16 or 17 or 18 or 19 or 20 or 21 or more nucleotides from position 9 of the complement of SEQ ID NO: 11 may be added to SEQ ID NO: 15. Similar considerations apply to extended versions of primers set forth in SEQ ID Nos: 18-20.

In another example, 12-mer primers comprising SEQ ID NO: 16 and SEQ ID NO: 17 are employed. Again, 3′-extended primers of SEQ ID NO: 16 and 17 may be produced and used without difficulty.

As will be known to the skilled artisan, a “primer” may comprise any combination of ribonucleotides, deoxyribonucleotides and analogs thereof such that it comprises DNA, RNA or DNA/RNA with one or more ribonucleotide or deoxyribonucleotide analogs contained therein, and capable of annealing to a nucleic acid template to act as a binding site for an enzyme, e.g., DNA or RNA polymerase, thereby providing a site for initiation of replication of a specific nucleic acid in the 5′ to 3′ direction. The nucleotide sequence of a primer is generally substantially complementary to the nucleotide sequence of a template nucleic acid to be amplified, or at least comprises a region of complementarity sufficient for annealing to occur and extension in the 5′ to 3′ direction there from. However, as will be apparent to the skilled artisan a degree of non-complementarity will not significantly adversely affect the ability of a primer to initiate extension. Suitable methods for designing and/or producing a primer suitable for use in the method of the invention are known in the art and/or described herein. Primers are generally, but not necessarily, short synthetic nucleic acids of about 12-50 nucleotides in length. Preferably, the first primer or each primer of the set of first primers comprises at least about 12-15 nucleotides in length capable of annealing to a strand of the nucleic acid template. Primers may also comprise at least about 20 or 25 or 30 nucleotides in length capable of annealing to a strand of the template.

As will be apparent to the skilled artisan, the number of nucleotides capable of annealing to a nucleic acid template is related to the stringency under which the primer will anneal. Thus, nucleotide mismatches between the primer and template are tolerated. Preferably, a primer for use in the method of the invention anneals to a nucleic acid template under moderate to high stringency conditions. The specific sequence of a primer for amplifying Ch1 d synthase-encoding nucleic acid of the invention will have sufficient identity to the target sequence to allow for annealing and initiation of an amplification reaction. In one example, a primer will have at least about 80% sequence identity overall to a region of a strand of a target template nucleic acid. More preferably, the degree of sequence identity is at least about 80% or 85% or 90% or 95% or 98% or 99% or 100%. In cases where the primer does not comprise a perfect match to the target sequence, the stringency of the annealing reaction may be lowered e.g., by reducing the annealing temperature, Stringency under which a primer anneals to a template nucleic acid may be determined empirically or in silico wherein the nearest neighbour method described by Breslauer et al., Proc. Natl. Acad. Sci. USA, 83, 3746-3750 (1986) may be employed to determine the Tm of the primer.

In the present context, the term “annealing” or similar term shall be taken to mean that a primer and a nucleic acid to be amplified (i.e., template or primary amplification product) are base-paired to each other to form a double-stranded or partially double-stranded nucleic acid, using a temperature or other reaction condition known in the art to promote or permit base-pairing between complementary nucleotide residues. As will be known to the skilled artisan, the ability to form a duplex and/or the stability of a formed duplex depend on one or more factors including the length of a region of complementarity between the primer and nucleic acid to be amplified, the percentage content of adenine and thymine in a region of complementarity (i.e., “A+T content”), the incubation temperature relative to the melting temperature (Tm) of a duplex, and the salt concentration of a buffer or other solution in which the amplification is performed. Generally, to promote annealing, the primers and nucleic acid to be amplified are incubated at a temperature that is at least about 1-5° C. below a primer Tm that is predicted from its A+T content and length. Duplex formation can also be enhanced or stabilized by increasing the amount of a salt (e.g., NaCl, MgCl₂, KCl, sodium citrate, etc) in the reaction buffer, or by increasing the time period of the incubation, as described by Sambrook et al., Molecular Cloning: A Laboratory Manual, Cold Spring Harbor Laboratory Press; Hames and Higgins, Nucleic Acid Hybridization: A Practical Approach, IRL Press, Oxford (1985); Berger and Kimmel, Guide to Molecular Cloning Techniques, In: Methods in Enzymology, Vol 152, Academic Press, San Diego Calif. (1987); or Ausubel et al., Current Protocols in Molecular Biology, Wiley Interscience, ISBN 047150338 (1992).

As a primer is generally extended in the 5′- to 3′-direction it is preferred that at least the 3′-terminal nucleotide is complementary to the relevant nucleotide in the template nucleic acid. More preferably, at least 3 or 4 or 5 or 6 or 7 or 8 or 9 or 10 contiguous nucleotides at the 3′-terminus of the primer are complementary to nucleotide sequence in the template nucleic acid. The complementarity of the 3′ terminus of the primer ensures that the extending end of the primer is capable of initiating amplification of the template nucleic acid, for example, by a polymerase.

As regions of non-complementarity reduce the predicted Tm of a primer and may be associated with amplification of non-template nucleic acid it is preferred that a primer of the invention does not comprise multiple contiguous nucleotides that are not identical to a strand of the template nucleic acid. Preferably, the primer comprises no more than 6 or 5 or 4 or 3 or 2 contiguous nucleotides that are not identical to a strand of the template nucleic acid. More preferably, any nucleotides that are not identical to a strand of the template nucleic acid are non-contiguous.

The primers set forth in SEQ ID NO: 14 or 15 or 16 or 17 or 18 or 19 or 20 or any extension product thereof may comprise one or modified bases, such as, for example, locked nucleic acid (LNA) or peptide nucleic acid (PNA). In such circumstances, shorter primers e.g., 8-mers or 9-mers or 10-mers or 11-mers or 12-mers may be employed without difficulty.

For the purposes of defining a level of stringency suitable for annealing a primer to a target template nucleic acid, a moderate stringency annealing conditions will generally be achieved at an incubation temperature between about 42° C. and about 55° C. and/or at a temperature that is between about 15° C. and 10° C. less than the predicted Tm for the primer, and in a reaction mixture comprising about 2 mM Mg²′ ion to about 3 mM Mg²⁺ ion concentration. High stringency annealing conditions will generally be achieved at an incubation temperature above about 55° C. and/or at a temperature that is between about 10° C. and 1° C. less than the predicted Tm for the primer, and in a reaction mixture comprising about 1 mM Mg²⁺ ion to about 2 mM Mg²⁺ ion concentration.

The Tm of a primer may be calculated using the method of Wallace et al., Nucleic Acids Res. 6, p3543, (1979), based on the G, C, T and A content. In particular, the described method uses the formula 2(A+G)+4(G+C) to estimate the Tm of a probe or primer. Alternatively, the nearest neighbour method described by Breslauer et al., Proc. Natl. Acad. Sci. USA, 83. 3746-3750 (1986) is employed, as follows:

Tₘ(calc)=ΣΔH⁰/(R ln(Cₜ/n)+ΣΔS⁰)

wherein ΔH⁰ is standard enthalpy for helix formation, ΔS⁰ is standard entropy for helix formation, Cₜ is the total strand concentration, n reflects the symmetry factor, which is 1 in the case of self-complementary strands and 4 in the case of non-self-complementary strands and R is the gas constant (1.987).

Ryuchlik et al., Nucl. Acids Res. 18: 6409-6412, 1990 described an alternative formula for determining Tm of an oligonucleotide:

$${Tm}^{primer} = \frac{d H}{d S + R ln ( c / 4 )} + 16.6 lg \frac{[ {K}^{*} ]}{1 + 0.7 [ {K}^{*} ]} - 273.15 ,$$

wherein, dH is enthalpy for helix formation, dS is entropy for helix formation, R is molar gas constant (1.987 cal/° C. mol), “c” is the nucleic acid molar concentration (determined empirically, W. Rychlik et. al., supra), (default value is 0.2 μM for unified thermodynamic parameters), [K⁺] is salt molar concentration (default value is 50 mM).

Suitable software for determining the Tm of an oligonucleotide using the nearest neighbour method is known in the art and available from, for example, US Department of Commerce, Northwest Fisheries Service Center and Department of Molecular Genetics and Biochemistry, University of Pittsburgh School of Medicine.

Alternatively, for longer primers (i.e., a primer comprising at least about 200 nucleotides), the method of Meinkoth et al. Anal Biochem, 138, 267-284 (1984), is useful for determining the Tm of the primer. This method uses the formula:

81.5+16.6(log₁₀M)+0.41(% GC)−0.61(% form)−500/Length in bp,

wherein M is the molarity of Na+ and % form is the percentage of formamide (set to 50%).

For a primer that comprises or consists of PNA, the Tm is determined using the formula (described by Giesen et al., Nucl. Acids Res., 26: 5004-5006):

T_{mpred}=c₀+c₁*T_{mnnDNA}+c₂*f_{pyr}+c₃*length,

wherein, in which T_{mnnDNA} is the melting temperature as calculated using a nearest neighbour model for the corresponding DNA/DNA duplex applying ΔH⁰ and ΔS⁰ values as described by SantaLucia et al. Biochemistry, 35; 3555-3562, 1995. f_{pyr} denotes the fractional pyrimidine content, and length is the PNA sequence length in bases. The constants are c₀=20.79, c₁=0.83, c₂=−26.13 and c₃=0.44.

To determine the Tm of a primer comprising one or more LNA residues a modified form of the formula of SantaLucia et al. Biochemistry, 35: 3555-3562, 1995 is used:

$$Tm = \frac{Δ H}{Δ S + ln ( [ Na ] \text{?} ( C / 4 ) \text{?} )} , \\ \text{?} \text{indicates text missing or illegible when filed}$$

A suitable program for determining the Tm of a primer comprising LNA is available from, for example, Exiqon, Vedbaek, Germany.

Preferably, a primer of the invention selectively anneals to a target nucleic acid. The term “selectively anneals” means that the probe is used under conditions where a target nucleic acid, anneals to the probe to produce a signal that is significantly above background (i.e., a high signal-to-noise ratio). The level of specificity of annealing is determined, for example, by performing an amplification reaction using the primer and detecting the number of different amplicons produced. By “different amplicons” is meant that amplified nucleic acids of differing nucleotide sequence and/or molecular weight are produced. Clearly, amplicons that differ in molecular weight are readily identified, for example, using gel electrophoresis. A primer that selectively anneals to a target nucleic acid produces an amplicon at a level greater than any other amplicon. Preferably only one amplicon is produced at a detectable level.

Methods for designing and/or selecting a primer suitable for use in an amplification reaction are known in the art and described, for example, in Innis and Gelfand (1990) (In: Optimization of PCRs. pp. 3-12 in: PCR Protocols (Innis, Gelfand, Sninsky and White, eds.); Academic Press, New York) and Dieffenbach and Dveksler (Eds) (In: PCR Primer: A Laboratory Manual, Cold Spring Harbour Laboratories, NY, 1995). Several software programs are available e.g., Primer3, available from the Center for Genome Research, Cambridge, Mass., USA, designs one or more primers for use in an amplification reaction based upon a known template sequence; Primer Premier 5, available from Biosoft International, Palo Alto, Calif., USA, designs and/or analyzes primers; CODEHOP, available from Fred Hutchinson Cancer Research Centre, Seattle, Wash., USA, designs primers based on multiple protein alignments; and FastPCR, available from Institute of Biotechnology, University of Helsinki, Finland, designs multiple primers, including primers for use in a multiplex reaction, based on one or more known sequences.

Methods for producing/synthesizing a primer of the present invention are known in the art. For example, oligonucleotide synthesis is described, in Gait (Ed) (In: Oligonucleotide Synthesis: A Practical Approach, IRL Press, Oxford, 1984). For example, a probe or primer may be obtained by biological synthesis (eg. by digestion of a nucleic acid with a restriction endonuclease) or by chemical synthesis. For short sequences (up to about 100 nucleotides) chemical synthesis is preferable. In one embodiment, a nucleotide comprising deoxynucleotides (e.g., a DNA based oligonucleotide) is produced using standard solid-phase phosphoramidite chemistry. Other methods for oligonucleotide synthesis include, for example, phosphotriester and phosphodiester methods (Narang, et al. Meth. Enzymol 68: 90, 1979) and synthesis on a support (Beaucage, et al Tetrahedron Letters 22: 1859-1862, 1981), and others described in “Synthesis and Applications of DNA and RNA,” S. A. Narang, editor, Academic Press, New York, 1987, and the references contained therein. For longer sequences standard replication methods employed in molecular biology are useful, such as, for example, the use of M13 for single stranded DNA as described by J. Messing (1983) Methods Enzymol, 101, 20-78.

As discussed supra a primer of the invention, or for use in the method of the invention may also include one or more nucleic acid analogs. For example, a primer comprises a phosphate ester analog and/or a pentose sugar analog. Alternatively, or in addition, a primer of the invention comprises polynucleotide in which the phosphate ester and/or sugar phosphate ester linkages are replaced with other types of linkages, such as N-(2-aminoethyl)-glycine amides and other amides (see, e.g., Nielsen et al., Science 254: 1497-1500, 1991; WO 92/20702; and U.S. Pat. No. 5,719,262); morpholinos (see, for example, U.S. Pat. No. 5,698,685); carbamates (for example, as described in Stirchak & Summerton, J. Org. Chem. 52: 4202, 1987); methylene(methylimino) (as described, for example, in Vasseur et al., J. Am. Chem. Soc. 114: 4006, 1992); 3′-thioformacetals (see, for example, Jones et al., J. Org. Chem. 58: 2983, 1993); sulfamates (as described, for example in, U.S. Pat. No. 5,470,967); 2-aminoethylglycine, commonly referred to as PNA (see, for example, WO 92/20702). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate. Methods for the production of a primer comprising such a modified nucleotide or nucleotide linkage are known in the art and discussed in the documents referred to supra. Exemplary primers comprise one or more LNA and/or PNA residues. Methods for the synthesis of an oligonucleotide comprising LNA are described, for example, in Nielsen et al, J. Chem. Soc. Perkin Trans., 1: 3423, 1997; Singh and Wengel, Chem. Commun. 1247, 1998. Methods for the synthesis of an oligonucleotide comprising are described, for example, in Egholm et al., Am. Chem. Soc., 114: 1895, 1992; Egholm et al., Nature, 365: 566, 1993; and Orum et al., Nucl. Acids Res., 21: 5332, 1993.

Any NASBA may be employed to isolate a Ch1 d synthase-encoding gene of theprsetn invention, including e.g., PCR-based amplification, reverse-transcriptase dependent PCT (RT-PCR) employing RNA as a template, an isothermal amplification method that does not require thermal cycling, or an amplification method performed in real time e.g., real time PCR or real-time RT-PCR or real-time isothermal amplification. Such processes may be performed with one or more labelled reporters that bind to the primers e.g., SYBR-green I and/or II, or with probes that bind to amplicons e.g., scorpion probes, TaqMan probes or molecular beacons.

In another example, a variant of SEQ ID NO: 11 or other A. marina sequence hereof comprises a nucleotide sequence that encodes a functional Ch1 d synthase polypeptide wherein said nucleotide sequence hybridizes under at least moderate stringency conditions to the complement of SEQ ID NO: 11 or other A. marina sequence hereof or a variant thereof comprising substitutions based on codon usage or on the degeneracy of the genetic code or a fragment thereof. Such variants include e.g., a protein-encoding part of a cDNA or genomic gene that comprises sufficient nucleotide sequence to encode a polypeptide that is functional in producing Ch1 d in a cell or organelle such as a chloroplast, or in a cell part e.g., thylakoid membrane. Genomic gene equivalents of SEQ ID NO: 11 o or other A. marina sequence hereof or variants thereof isolated from other organisms are also encompassed e.g., wherein the protein-encoding sequence is operably connected to or placed operably in connection with one or more regulatory sequences such as a promoter sequence and/or a transcriptional terminator sequence.

The term “fragment” in this context means a hybridization probe that comprises a sequence that is complementary to 10 or 15 or 20 or 25 or 30 or 35 or 40 or 45 or 50 or 55 or 60 or 65 or 70 or 75 or 80 or 85 or 90 or 95 or 100 or more contiguous nucleotides of SEQ ID NO: 11 or other exemplified A. marina sequence hereof, or a variant thereof comprising substitutions based on codon usage or on the degeneracy of the genetic code. There is no restriction on the precise sequence of a fragment to be employed in this context, subject to the fragment being of a sufficient length for specific hybridization to a variant Ch1 d synthase-encoding gene to occur. As with amplification, longer probes are preferred for specific nucleic acid hybridization.

In one example, fragments of the Ch1 d synthase-encoding gene for isolation of variants are produced by an art-recognized NASBA. For example, a probe produced by amplification of A. marina nucleic acid employing primers comprising SEQ ID NO: 5 and SEQ ID NO: 6 may be employed. In this case, the probe will comprise a nucleotide sequence that is complementary to SEQ ID NO: 13. Alternatively, or in addition, a probe produced by amplification of A. marina nucleic acid employing primers comprising SEQ ID NOs: 16 and SEQ ID NO: 6 may be employed. Alternatively, or in addition, a probe produced by amplification of A. marina nucleic acid employing primers comprising SEQ ID NOs: 5 and SEQ ID NO: 17 may be employed. Alternatively, or in addition, a probe produced by amplification of A. marina nucleic acid employing primers comprising SEQ ID NOs: 16 and SEQ ID NO: 17 may be employed. Alternatively, or in addition, a probe produced by amplification of A. marina nucleic acid employing primers comprising SEQ ID NOs: 18 and SEQ ID NO: 20 may be employed. Alternatively, or in addition, a probe produced by amplification of A. marina nucleic acid employing primers comprising SEQ ID NOs: 19 and SEQ ID NO: 20 may be employed.

In another example, fragments of the Ch1 d synthase-encoding gene for isolation of variants are produced by restriction enzyme digestion of the full-length A. marina Ch1 d synthase open reading frame i.e., SEQ ID NO: 11 or other A. marina sequence hereof. All such fragments are employed to isolate variants including genomic gene equivalents of SEQ ID NO: 11 or other A. marina sequence hereof and homologous genes from species other than A. marina by standard nucleic acid hybridization reactions without undue experimental effort.

Fragments of the Ch1 d synthase-encoding gene for isolation of variants may be produced by replication of phagemid vector comprising double-stranded nucleic acid e.g., comprising the full-length A. marina Ch1 d synthase open reading frame or any fragment thereof of at least 10 nucleotides in length such as double-stranded nucleic acid comprising the sequence of SEQ ID NO: 13, to thereby produce a single-stranded probe complementary to SEQ ID NO: 11 or other A. marina sequence hereof.

In each and every foregoing example, of variant Ch1 d synthase-encoding genes and variant Ch1 d synthase polypeptides, the variant may comprise a sequence having at least about 70% identity overall at the nucleotide level to SEQ ID NO: 11 and/or at least about 70% identity overall at the amino acid level to SEQ ED NO: 12. Preferred levels of sequence, identity to SEQ ID NO: 11 or 12 will be at least about 75% or at least about 80% at least about 85% at least about 90% at least about 91% at least about 92% at least about 93% at least about 94% at least about 95% at least about 96% at least about 97% at least about 98% at least about or 99%. Exemplary nucleic acid variants of SEQ ID NO: 11 falling within these limits are set forth herein as SEQ ID NO: 21, SEQ ID NO: 23, SEQ ID NO: 25 and SEQ ID NO: 28 and the corresponding encoded polypeptide variants are set forth herein as SEQ ID NO: 22, SEQ ID NO: 24, SEQ ID NO: 26 and SEQ ID NO: 28 respectively.

To determine whether or not two nucleotide sequences fall within a particular percentage identity limitation recited herein, those skilled in the art will be aware that it is necessary to conduct a side-by-side comparison or multiple alignment of sequences. In such comparisons or alignments, differences may arise in the positioning of non-identical residues, depending upon the algorithm used to perform the alignment. In the present context, reference to a percentage identity between two or more nucleotide sequences shall be taken to refer to the number of identical residues between said sequences as determined using any standard algorithm known to those skilled in the art. For example, nucleotide sequences may be aligned and their identity calculated using the BESTFIT program or other appropriate program of the Computer Genetics Group, Inc., University Research Park, Madison, Wis., United States of America (Devereaux et al., Nucl. Acids Res. 12, 387-395, 1984).

Alternatively, a suite of commonly used and freely available sequence comparison algorithms is provided by the National Center for Biotechnology Information (NCBI) Basic Local Alignment Search Tool (BLAST) (Altschul et al. J. Mol. Biol. 215: 403-410, 1990), which is available from several sources, including the NCBI, Bethesda, Md. The BLAST software suite includes various sequence analysis programs including “blastn,” that is used to align a known nucleotide sequence with other polynucleotide sequences from a variety of databases. Also available is a tool called “BLAST 2 Sequences” that is used for direct pairwise comparison of two nucleotide sequences. As used herein the term “NCBI” shall be taken to mean the database of the National Center for Biotechnology Information at the National Library of Medicine at the National Institutes of Health of the Government of the United States of America, Bethesda, Md., 20894,

### 2. Gene Constructs for Expressing Functional Ch1 d Synthase Polypeptide

For expressing Ch1 d synthase protein by recombinant means, a protein-encoding nucleic acid e.g., comprising a minimum open reading frame sufficient for encoding a functional protein is placed in operable connection with a 5′ non-coding region such as a promoter or other regulatory sequence capable of regulating expression in a cell-free system or cellular system. For example, nucleic acid comprising a sequence that encodes a peptide is placed in operable connection with a suitable promoter and maintained in a suitable cell for a time and under conditions sufficient for expression to occur. Nucleic acid encoding Ch1 d synthase polypeptides is described herein.

An “expression construct” comprising the Ch1 d synthase-encoding gene and a suitable promoter is produced according to any art-recognized method such that the promoter is capable of conferring expression or a pattern of expression on said Ch1 d synthase-encoding gene e.g., in green tissues of a plant or in a bacterial cell or cyanobacterial cell or a chloroplast. For example, any cell, tissue or organ having the ability to confer expression on the nucleic acid to which the promoter is operably-connected may be transformed with the expression construct.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid (e.g., a transgene), e.g., in response to a developmental and/or external stimulus, or in a tissue specific manner. In the present context, the term “promoter” is also used to describe a recombinant, synthetic or fusion nucleic acid, or variant which confers, activates or enhances the expression of a nucleic acid (e.g., a transgene and/or a selectable marker gene and/or a detectable marker gene) to which it is operably linked. Preferred promoters can contain additional copies of one or more specific regulatory elements to further enhance expression and/or alter the spatial expression and/or temporal expression of said nucleic acid.

As used herein, the term “in operable connection with”, “in connection with” or “operably linked to” means positioning a promoter relative to a nucleic acid (e.g., a transgene) such that expression of the nucleic acid is controlled by the promoter. For example, a promoter is generally positioned 5′ i.e., upstream of the nucleic acid encoding Ch1 d synthase protein, the expression of which it controls. To construct heterologous promoter/nucleic acid combinations, it is generally preferred to position the promoter at a distance from the gene transcription start site that is approximately the same as the distance between that promoter and the nucleic acid it controls in its natural setting, i.e., the gene from which the promoter is derived. As is known in the art, some variation in this distance can be accommodated without loss of promoter function.

For expression of Ch1 d synthase protein in vitro, a suitable promoter includes, but is not limited to T3 promoter, SP6 promoter, T7 promoter e.g., Hanes and Plückthun, Proc. Natl. Acad. Sci. USA, 94, 4937-4942 (1997). Typical expression vectors for expression in vitro or cell-free have been described and include, but are not limited to the TNT T7 and TNT T3 systems (Promega), the pEXP1-DEST and pEXP2-DEST vectors (Invitrogen).

Typical promoters suitable for expression in bacterial cells include, but are not limited to, the lacZ promoter, the Ipp promoter, temperature-sensitive λL promoter or λR promoter, the T7 promoter, the T3 promoter, the SP6 promoter, or semi-artificial promoters such as the IPTG-inducible tac promoter or lacUV5 promoter. A number of other gene construct systems for expressing the nucleic acid fragment of the invention in bacterial cells are well-known in the art and are described for example, in Ausubel et al. (In: Current Protocols in Molecular Biology. Wiley Interscience, ISBN 047 150338, 1987), U.S. Pat. No. 5,763,239 (Diversa Corporation) and Sambrook et al. (In: Molecular Cloning: Molecular Cloning: A Laboratory Manual, Cold Spring Harbor Laboratories, New York, Third Edition 2001).

Numerous expression vectors for expression of recombinant polypeptides in bacterial cells and efficient ribosome binding sites have also been described, and include, for example, PKC30 e.g., Shimatake and Rosenberg, Nature 292, p128 (1981); pKK173-3 e.g., Amann and Brosius, Gene 40, p 183 (1985), pET-3 (Studier and Moffat, J. Mol. Biol. 189, p113 (1986); the pCR vector suite (Invitrogen), pGEM-T Easy vectors (Promega), the pL expression vector suite (Invitrogen) the pBAD/TOPO or pBAD/thio-TOPO series of vectors containing an arabinose-inducible promoter (Invitrogen, Carlsbad, Calif.); the pFLEX series of expression vectors (Pfizer Inc., CT, USA); the pQE series of expression vectors (QIAGEN, CA, USA), or the pL series of expression vectors (Invitrogen), amongst others.

For expression in the nuclei of higher plants and green algae, the well-known ubiquitin promoter or CaMV 19S promoter or CaMV 35S promoter may be employed conveniently. In general, promoters for selective expression in the green tissues of monocotyledonous plants and dicotyledonous plants may, differ. Exemplary monocot promoters for expressing Ch1 d synthase in green tissues of monocot plants will be isolated from a light harvesting chlorophyll a/b binding protein-encoding gene of a monocot species e.g., Zea mays Cab-m⁷ gene promoter or Oryza saliva Cab1R gene promoter, or from a wheat nuclear-encoded protochlorophyllide reductase-encoding gene or barley NADPH-protochlorophyllide oxidoreductase-encoding gene. Exemplary dicot promoters for expressing Ch1 d synthase in green tissues of dicot plants will be isolated from a dicot nuclear-encoded protochlorophyllide reductase-encoding gene e.g., Pisum sativum lpcr gene promoter, or from a dicot ribulose 1,5-bisphosphate carboxylase-encoding gene e.g., Arabidopsis thaliana ats1B or ats3B gene promoter or Petunia hybrida rbcS gene promoter or P. sativum rbcS-3A or rbcS-3C gene promoter or Brassica napus rbcSF1 gene promoter, or from a dicot chlorophyll a/b binding protein-encoding gene e.g., Nicotiana plumbaginifolia Cab-E gene or Nicotiana sylvestris Lhcb1*3 or Lhcb1*8 or Lhcb1*9 gene promoter or Piston sativum LHC AB80 gene promoter, or from a dicot NADPH-protochlorophyllide oxidoreductase-encoding gene e.g., Cucumis sativus NPR gene or A. thaliana PorA or PorB or P or C gene, or from a dicot phytochrome biosynthetic gene e.g., Solarium tuberosum phyB gene, or from a chloroplastic ribosomal protein e.g., Spinacia oleracea rps1 gene promoter or GT3 promoter or Brassica napus oleosin BN-III gene promoter, or from a dicot photosystem gene promoter e.g., Spinacia oleracea psaF gene promoter. The promoter from the A. thaliana chloroplast-targeted regulatory factor gene i.e., CAO gene promoter, or the promoter from the Lycopersicon esculentum chloroplastic Cu, Zn superoxide dismutase-encoding gene, or the promoter from the Spinacia oleracea thylakoid-bound ascorbate peroxidise-encoding gene Apx2, or the promoter from the Phaseolus vulgaris plastid-located glutamine synthetase-encoding gene gln-delta, may also be employed. A database comprising approximately 300 experimentally-verified plant promoters developed by the Department of Computer Science at Royal Holloway, University of London, in collaboration with Softberry Inc. (USA), is publicly-available through the websites of either organization.

For expression in plastids and cyanobacteria, a promoter from a 16S rRNA gene or pcbA gene or Prrn gene or Synechocystis NADPH-protochlorophyllide oxidoreductase gene may be employed.

Preferred linkages between the promoter and the Ch1 d synthase-encoding gene are covalent linkages. It is to be understood that, because the promoter, active fragment or derivative may confer expression at some distance from the Ch1 d synthase-encoding gene to which it is operably connected, the gene need not be juxtaposed to the promoter, i.e., there may be intervening sequence of up to about 2 kb in length, preferably up to about 1 kb in length, more commonly about 200-500 bp in length. Shorter intervening sequences such as the sequence of an intron of up to about 100 or 200 bp in length may also be employed. Suitable methods for linking nucleic acids will be apparent to the skilled artisan and/or described herein and include enzymatic ligation, e.g., T4 DNA ligase, topoisomerase-mediated ligation e.g., using Vaccinia DNA topoisomerase I, recombination in cis or trans, e.g., using a recombinase or by random integration, amplification from one or more primer sequences including primer extension means, amplification from a vector, or chemical ligation, e.g., cyanogen bromide-mediated condensation of nucleic acids.

The skilled artisan will be aware that an expression construct may also comprise one or more transcription termination sequences e.g., CaMV 35S or CaMV 19S or Ubi or rps16 or rbcL or pcbA or petD gene terminator and/or intron sequences with flanking intron splice junction sequences e.g., the intron 1 sequence from a dicot or monocot ubiquitin (Ubi) gene.

An expression construct will generally comprise one or more origins of replication. The specific structure of an origin of replication will vary between species, however will generally comprise a high A+T nucleotide content e.g., to permit binding to a pre-replication complex and unwinding of a nucleic acid duplex. The origin of replication may have a narrow host range that is operable only in a limited number of different species or a single species, or alternatively, it may have a broad host range operable across different species of the same kingdom. Broad host range origins of replication e.g., on pSa, are preferred for general applications. The origin of replication may result in maintenance of the gene construct of the invention at high copy number e.g., more than 50 or more than 100 or more than 250 or more than 500 or more than 1000 copies per cell or organelle, or it may be maintain the gene construct at a low copy number e.g., less than 50 or less than 25 copies per cell or organelle.

In one example, the origin of replication is operable in a prokaryote e.g., a bacterium (e.g., colE1, pMB1, p15A, pSC101, oriC, pVS1, pTAR or pRiHR1) or a cyanobacterium (e.g., pFdA from F. diplosiphon, pDU1 from Nostoc sp. strain PCC 7524, or pANS from Anacystis nidulans. In another example, the origin of replication is operable in a eukaryote e.g., a yeast, such as an autonomous replicating sequence (ARS) and elements comprising an ARS of yeasts or green algae such as C. reinhardii. DNA Replication Origin Database (OriDB; Nieduszynski, University of Aberdeen, Scotland). In another example, the origin of replication is operable in a chloroplast of a plant of algal cell, such as the oriA sequence amongst others. See e.g., Kunnimalaiyaan et al., Nucleic Acids Res 25, 3681-3686 (1997); Lau et al., FEMS Microbiol. Lett. 27, 253-256 (2003); Lugo et al., Plant Sci 166, 151-161 (2004); Schaefer et al., J. Bacteriol, 175, 5701-5705 (1993); Schmetterer et al., Gene 62, 101-109 (1988); Vallet et al., Curr. Genet. 9, 321-324 (1985); Verma et al., Plant Physiology 145, 1129-1143 (2007); Wolk et al., Proc. Natl. Acad. Sci. (USA) 81, 1561-1565 (1984); and DNA Replication Origin Database (OriDB; Nieduszynski, University of Aberdeen, Scotland). The use of a plurality of origins of replication such that the gene construct is able to be maintained in prokaryotic and eukaryotic cells, or in prokaryotic cells and in chloroplasts, or in eukaryotic cells and in chloroplasts, or in prokaryotic and eukaryotic cells and chloroplasts, are also preferred.

An expression construct will generally also include one or more sequences to permit it to be maintained in a cell under selective conditions e.g., one or more selectable marker genes. As will be known to the skilled artisan, a selectable marker gene may confer antibiotic or herbicide resistance on cells comprising a gene construct that expresses the selectable marker gene. Preferred selectable marker genes include the bacterial bar gene and/or a bacterial neomycin phosphotransferase II (nptII) gene and/or a hygromycin phosphotransferase gene and/or an aacC3 gene and/or aacC4 gene and/or a chloramphenicol acetyl transferase (CAT) gene and/or a gene encoding 5-enolpyruvyl-shikimate-3-phosphate synthase and/or a gene encoding phosphinothricin synthase and/or mana gene and/or cyanamide hydratase (Cah) gene and/or D-amino oxidase, (DAAO) gene and/or an ampicillin resistance gene and/or a spectinomycin resistance gene and/or an aminoglycoside 3′-adenylyltransferase gene and/or a betaine aldehyde dehydrogenase (badh) gene. In one example, a selectable marker gene is operably connected to a ubiquitous promoter e.g., a promoter from ubiquitin (ubi) or from the cauliflower mosaic virus, e.g., CaMV 35S. Suitable promoters and selectable markers will be apparent to the skilled artisan or are described herein.

An expression construct may also comprise additional components, such as, for example, a sequence encoding a targeting sequence and/or a detectable label. Such an additional component may be located between the promoter and the Ch1 d synthase-encoding gene, e.g., such that it is expressed as a 5′-fusion with the polypeptide encoded by the Ch1 d synthase-encoding gene. Alternatively, the additional component may be located 3′ to the Ch1 d synthase-encoding gene.

A targeting sequence is a sequence of amino acids within a polypeptide that directs the polypeptide to a particular subcellular location. Targeting sequences useful for the performance of the invention are known in the art and described by e.g., Bruce, Trends Cell Biol. 10, 440-447 (2000); Franzén et al., FEBS Lett. 260, 165-168 (1990); Johnson et al., The Plant Cell 2:525-532, 1990; Mueckler et al. Science 229, 941-945 (1985); Iturriaga et al. The Plant Cell 1, 381-390 (1989); McKnight et al., Nucl. Acid Res. 18, 4939-4943 (1990); Matsuoka and Nakamura, Proc. Natl. Acad. Sci. USA 88, 834-838 (1991); and In: “Recombinant proteins from plants”, Eds. C. Cunningham and A. J. R. Porter, 1998, Humana Press Totowa, N.J., USA. These publications permit the production of recombinant proteins that are targeted to different compartments of plant cells.

Suitable detectable markers include, for example, an epitope, e.g., influenza virus hemagglutinin (HA), Simian Virus 5 (V5), polyhistidine, c-myc, cholera toxin B-subunit or FLAG epitope.

An expression construct may also include one or more recombinase site sequences to permit excision of a portion of its DNA in a cell and/or to facilitate integration into host cell DNA. Exemplary flanking recombinase site sequences are selected from trnA, trnG, trnI, trnL, trnM, trnN, trnR, trnS, trnV, trnfM, rrn16, rbcL, rpI32, accD, petA, 3′rps7/12, ycf3 and psbJ.

For example, an expression constructs of the present invention may be contained within an expression vector e.g., for delivery to a target cell. In the case of an expression vector to be delivered into a plant using Agrobacterium-based transformation, the vector preferably comprises a left-border (LB) sequence and a right-border (RB) sequence that flank the transgene to be delivered into the plant cell, i.e., the transfer DNA. Such a vector may also comprise a suitable selectable marker for selection of bacteria comprising the vector, e.g., conferring resistance to ampicillin.

Preferred vectors for delivery to plants cells are binary Ti plasmids or Ri plasmids. Binary Ti plasmids or Ri plasmids are produced based on the observation that the T-DNA (nucleic acid transferred to a plant cell) and the vir genes required for transferring the T-DNA may reside on separate plasmids (Hoekema et al., Nature, 303: 179-180, 1983). In this respect, the vir function is generally provided by a disarmed Ti plasmid resident in or endogenous to the Agrobacterium strain used to transform a plant cell. Accordingly, a binary Ti plasmid or Ri plasmid comprises a transgene located within transfer-nucleic acid (e.g., T-DNA). Such transfer-nucleic acid comprising the transgene is generally flanked by or delineated by a LB and a RB.

Suitable binary plasmids are known in the art and/or commercially available. For example, a selection of binary Ti vectors includes pBIN 19 (Bevan et al., Nucleic Acids Res., 12: 8711-8721, 1984); pC22 (Simoens et al., Nucleic Acids Res. 14: 8073-8090, 1986); pGA482 (An et al., EMBO J. 4: 277-284, 1985); pPCV001 (Koncz and Schell Mol. Gen. Genet. 204: 383-396, 1986); pCGN 1547 (McBride and Summerfelt 14: 269-276, 1990); pJJ 1881 (Jones et al., Transgenic Res. 1: 285-297, 1992); pPZPIII (Hajukiewicz et al., Plant Mol. Biol., 25: 989-994, 1994); and pGreen0029 (Hellens et al., Plant Mol. Biol., 42: 819-832, 2000). Additional binary vectors are described in, for example, Hellens and Mullineaux Trends in Plant Science 5: 446-451, 2000. Variants of these plasmids e.g., as described herein or known in the art may also be employed. Suitable Ri plasmids are also known in the art and include, for example, pRiA4b (Juouanin Plasmid, 12: 91-102, 1984), pRi1724 (Moriguchi et al., J. Mol. Biol. 307:771-784, 2001), pRi2659 (Weller et al., Plant Pathol. 49:43-50, 2000) or pRi1855 (O'Connell et al., Plasmid 18:156-163, 1987). Such vectors will generally also comprise one or more selectable marker gene for conferring resistance against a herbicide on transformed plant cells. For example, a bacterial bar gene, encoding phosphinothricin acetyltransferase (PAT) and conferring resistance against bialaphos and/or a bacterial neomycin phosphotransferase II (nptII) gene conferring resistance against kanamycin and/or a hygromycin phosphotransferase gene conferring resistance against hygromycin and/or an aacC3 gene and/or aacC4 gene and/or a chloramphenicol acetyl transferase gene and/or a gene encoding 5-enolpyruvyl-shikimate-3-phosphate synthase and/or a gene encoding phosphinothricin synthase may be employed to confer resistance to a herbicide or an antibiotic. Alternatively, the reporter gene confers the ability to survive and/or grow in the presence of a compound in which an untransformed plant cell cannot grow and/or survive, e.g., a mana gene e.g., Hansen and Wright, Trends in Plant Sciences, 4: 226-231 (1999), a cyanamide hydratase (Cah) gene e.g., U.S. Ser. No. 09/518,988, or a D-amino oxidase, (DAAO) gene e.g., Erikson et al., Nature Biotechnology 22, 455-458 (2004) may be employed.

Chloroplast transformation vectors are also designed with homologous flanking sequences on either side of the transgene cassette to facilitate double recombination facilitated by a RecA-type system between the plastid-targeting sequences of the transformation vector and the targeted region of the plastid genome. Targeting sequences have no special properties other than that they are homologous to the chosen target site and are generally about 1 kb in size. Both flanking sequences are essential for homologous recombination. Chloroplast vectors may also carry an origin of replication e.g., oriA, that facilitates replication of the plasmid inside the chloroplast, thereby increasing the template copy number for homologous recombination and consequently enhancing the probability of transgene integration. Integration of transgenes between exons of trnA and trnI facilitates correct processing of foreign transcripts because of processing of introns present within both flanking regions.

Preferred selectable markers genes for chloroplast transformation are the spectinomycin resistance gene encoded by a mutant 16S ribosomal RNA (rRNA) gene, and the Chlamydomonas aadA gene encoding aminoglycoside 3′-adenylyltransferase that inactivates spectinomycin and streptomycin by adenylation thereby preventing it binding to chloroplast ribosomes. The neo gene and nptII gene and aphA6 gene confer kanamycin resistance on chloroplasts, and combinations of aphA6 and nptII may be employed wherein aphA6 expression is regulated by a 16S rRNA promoter and gene 10 UTR capable of expression in the dark and in non-green tissues and the nptII gene is regulated by a pcbA promoter and UTR capable of expression in the light, to thereby provide selection during day and night as well as in developing plastids and mature chloroplasts. Such a double-barrel transformation vector is more efficient than single gene e.g., aphA6-based chloroplast vectors. Alternatively, or in addition, a betaine aldehyde dehydrogenase (badh) gene may be employed to provide selection against the toxic substrate betaine aldehyde.

### 3. Transformation of Cells and Chloroplasts

Means for introducing recombinant DNA into cells, including plant cells include, but are not limited to, transformation using CaCl₂ and variations thereof, direct DNA uptake into protoplasts (Krens et al, Nature 296, 72-74, 1982; Paszkowski et al., EMBO J. 3, 2717-2722, 1984), PEG-Mediated uptake to protoplasts (Armstrong et al., Plant Cell Rep. 9, 335-339, 1990) microparticle bombardment, electroporation (Fromm et al., Proc. Natl. Acad. Sci. (USA), 82, 5824-5828, 1985), microinjection of DNA (Crossway et al., Mol. Gen. Genet. 202, 179-185, 1986), microparticle bombardment of tissue explants or cells (Christou et al. Plant Physiol. 87, 671-674, 1988; Sanford, Part. Sci. Technol. 5, 27-37, 1988), vacuum-infiltration of tissue with nucleic acid, or in the case of plants, T-DNA-mediated transfer from Agrobacterium to the plant tissue as described essentially by An et al., EMBO J. 4, 277-284, 1985; Herrera-Estella et al., Nature 303, 209-213, 1983; Herrera-Estella et al., EMBO J. 2, 987-995, 1983; or Herrera-Estella et al., In: Plant Genetic Engineering, Cambridge University Press, N.Y., pp 63-93, 1985.

Particle bombardment-mediated transformation also delivers naked nucleic acid into plant cells (Sanford et al., J. Part. Sci. Technol. 5, 27, 37, 1987). This technique involves the acceleration of dense nucleic acid-coated microparticles, e.g., gold or tungsten particles, to a sufficient velocity to penetrate the plant cell wall and nucleus. The introduced nucleic acid is then incorporated into the plant genome, thereby producing a transgenic plant cell. This cell is, then used to regenerate a transgenic plant. Exemplary apparatus and procedures are disclosed by Stomp et al. (U.S. Pat. No. 5,122,466) and Sanford and Wolf (U.S. Pat. No. 4,945,050). Examples of microparticles suitable for use in such systems include 1 to 5 micron gold spheres. The DNA construct may be deposited on the microparticle by any suitable technique, such as by precipitation.

Alternatively, an expression construct or expression vector is introduced into a plant protoplast. To produce a protoplast, it is necessary to remove the cell wall from a plant cell. Methods for producing protoplasts are known in the art and described, for example, by Potrykus and Shillito, Methods in Enzymology 118, 449-578, 1986. Naked nucleic acid (i.e., nucleic acid that is not contained within a carrier, vector, cell, bacteriophage or virus) is introduced into a plant protoplast by physical or chemical permeabilization of the plasma membrane of the protoplast (Lörz et al., Mol. Gen. Genet. 199, 178-182, 1985 and Fromm et al., Nature, 319, 791-793, 1986).

A preferred physical means for introducing nucleic acid into protoplasts is electroporation, which comprises the application of brief, high-voltage electric pulses to the protoplast, thereby forming nanometer-sized pores in the plasma membrane. Nucleic acid is taken up through these pores and into the cytoplasm. Alternatively, the nucleic acid may be taken up through the plasma membrane as a consequence of the redistribution of membrane components that accompanies closure of the pores. From the cytoplasm, the nucleic acid is transported to the nucleus where it is incorporated into the genome.

A preferred chemical means for introducing nucleic acid into protoplasts utilizes polyethylene glycol (PEG). PEG-mediated transformation generally comprises treating a protoplast with nucleic acid of interest in the presence of a PEG solution for a time and under conditions sufficient to permeabilize the plasma membranes of the protoplast. The nucleic acid is then taken up through pores produced in the plasma membrane and either maintained as an episomal plasmid or incorporated into the genome of the protoplast.

In another example, the expression vector or construct is introduced into a plant cell by electroporation (Fromm et al., Proc. Natl. Acad. Sci. USA 82, 5824, 1985). In this technique, plant protoplasts are electroporated in the presence of plasmids or nucleic acids containing the relevant genetic construct. Electrical impulses of high field strength reversibly permeabilize biomembranes allowing the introduction of the plasmids. Electroporated plant protoplasts reform the cell wall, divide, and form a plant callus. Selection of the transformed plant cells with the transformed gene can be accomplished using phenotypic markers.

Cauliflower mosaic virus (CaMV) is also useful as a vector for introducing an expression vector or construct into plant cells (Hohn et al., (1982) “Molecular Biology of Plant Tumors,” Academic Press, New York, pp. 549-560; Howell, U.S. Pat. No. 4,407,956). CaMV viral DNA genome is inserted into a parent bacterial plasmid creating a recombinant DNA molecule that can be propagated in bacteria. After cloning, the recombinant plasmid is again cloned and further modified by introduction of the desired nucleic acid. The modified viral portion of the recombinant plasmid is then excised from the parent bacterial plasmid, and used to inoculate the plant cells or plants.

A further method for introducing an expression construct into plant cells is to infect a plant cell, an explant, a meristem or a seed with Agrobacterium tumefaciens comprising the expression construct. Under appropriate conditions known in the art, the transformed plant cells are grown to form shoots, roots, and develop further into plants. The expression construct is introduced into appropriate plant cells, for example, by means of the Ti plasmid or Ri plasmid of Agrobacterium tumefaciens. The Ti plasmid is transmitted to plant cells upon infection by Agrobacterium tumefaciens, and is stably integrated into the plant genome (Horsch et al., Proc. Natl. Acad. Sci. USA 80, 4803, 1984).

There are presently at least three different ways to transform plant cells with Agrobacterium: (1) co-cultivation of Agrobacterium with cultured isolated protoplasts; (2) transformation of cells or tissues with Agrobacterium, or (3) transformation of seeds, apices or meristems with Agrobacterium. Method (1) uses an established culture system that allows culturing protoplasts and plant regeneration from cultured protoplasts. Method (2) implies (a) that the plant cells or tissues can be transformed by Agrobacterium and (b) that the transformed cells or tissues can be induced to regenerate into whole plants. Method (3) uses micropropagation. In the binary system, to have infection, two plasmids are needed: a T-DNA containing plasmid and a vir plasmid. Any one of a number of T-DNA containing plasmids can be used, the main issue being that one be able to select independently for each of the two plasmids.

After transformation of the plant cell or plant, those plant cells or plants transformed by the Ti plasmid so that the desired DNA segment is integrated can be selected by an appropriate phenotypic marker expressed by the transformation vector. These phenotypic markers include, but are not limited to, antibiotic resistance, herbicide resistance or a trait detectable by visual observation. Other phenotypic markers are known in the art and may be used in this invention.

Alternatively, the transformed plants are produced by an in planta transformation method using Agrobacterium tumefaciens, such as, for example, the method described by Bechtold et al., CR Acad. Sci. (Paris, Sciences de la vie/Life Sciences) 316, 1194-1199, 1993 or Clough et al., Plant J 16, 735-74, 1998, wherein A. tumefaciens is applied to the outside of the developing flower bud and the binary vector DNA is then introduced to the developing microspore and/or macrospore and/or the developing seed, so as to produce a transformed seed. Those skilled in the art will be aware that the selection of tissue for use in such a procedure may vary, however it is preferable generally to use plant material at the zygote formation stage for in planta transformation procedures.

In a further example, a graminaceous plant is transformed using a method comprising contacting a mature embryo, e.g., a wheat embryo from a seed that has completed grain filling, with an Agrobacterium comprising an expression vector for a time and under conditions sufficient for the expression vector to be delivered to one or more cells of the mature embryo. Such transformation may additionally comprise removing the seed coat and or performing the transformation in the presence of Soytone™, both of which improve transformation efficiency. The transformed cells may be used to regenerate a plant or plant part.

The present invention also encompasses products of repeated cycles of transformation employing transformed plant cells or plant parts comprising a promoter, active fragment or derivative of the present invention or a transgene placed operably under the control of said promoter, active fragment or derivative or a gene construct comprising said transgene operably under the control of said promoter, active fragment or derivative.

In one example, gene stacking is performed sequentially or simultaneously. In one example of simultaneous gene stacking, a plant cell, plant tissue, plant organ or whole plant is transformed with two gene constructs wherein at least one of said gene constructs comprises a gene construct of the present invention i.e., comprising the Ch1 d synthase-encoding gene.

Plastid transformation is typically based on DNA delivery by the biolistic process or occasionally by polyethylene glycol (PEG) treatment of protoplasts. For example, plastid transformation is most commonly achieved by biolistic delivery of DNA into leaf explants.

A variety of approaches can be employed for transformation of Chlamydomonas reinhardtii cells, Transformation of the Chlamydomonas reinhardtii nuclear compartment is performed as described e.g., by Kindle et al., Proc Natl Acad Sci 87, 1228-1232 (1990). In brief, cells are grown to a concentration of 1.5×106 per ml, pelleted, and resuspended in 5% (v/v) polyethylene glycol (PEG, Mr 6000) from Sigma-Aldrich (St. Louis, Mo.). Subsequently, the resuspended cells are incubated with linear or circular plasmid DNA and agitated in the presence of 300 mg of 0.5 mm glass beads for 10-30 seconds at maximum speed using a Genie Vortex II mixer (Thermo Fisher Scientific, Pittsburgh, Pa.), Cells are immediately plated on agarose plates containing the appropriate antibiotic or nutrient selection. Alternately, Chlamydomonas reinhardtii cells are plated directly onto agarose plates and bombarded with 500 μg of tungsten microprojectiles coated with 1 μg of plasmid DNA using the Bio-Rad PDS-IOOOHe particle-delivery system (Bio-Rad Laboratories; Hercules, Calif.), as previously described e.g., Sodeinde et al., Proc Natl Acad Sci 90, 9199-9203 (1993).

Chlamydomonas reinhardtii chloroplasts are transformed as described e.g., Kindle et al., Proc Natl Acad Sci. 88, 1721-1725 (2001). In brief, cells are grown to mid log phase, optionally in the presence of 0.5 mM 5-fluorodeoxyuridine, and agitated with single or double-stranded plasmid DNA in the presence of 300 mg of 0.5 mm glass beads for 15-30 seconds at maximum speed using a Genie Vortex II mixer. Following agitation, cells are immediately plated on selective agar plates containing the appropriate concentration of antibiotics. Typically, 100 μg/ml of spectinomycin is used.

4. Regeneration of Plants from Transformed Cells

A whole plant may be regenerated from the transformed or transfected cell, in accordance with procedures known in the art. Plant tissue capable of subsequent clonally propagation, whether by organogenesis or embryogenesis, may be transformed with a vector or construct as described herein according to any embodiment.

The term “organogenesis”, as used herein, means a process by which shoots and roots are developed sequentially from meristematic centres.

The term “embryogenesis”, as used herein, means a process by which shoots and roots develop together in a concerted fashion (not sequentially), whether from somatic cells or gametes.

Plant regeneration from cultural protoplasts is described, for example, in Evans et al., “Protoplast Isolation and Culture—Handbook of Plant Cell Cultures 1” (MacMillan Publishing Co., 1983) and Binding “Regeneration of Plants”—Plant Protoplasts, pp 21-73 (CRC Press, Boca Raton, 1985). Regeneration varies from species to species. Generally, a suspension of transformed protoplasts is produced (e.g., using a method described herein). In some species the transformed protoplast is then induced to form an embryo and then to the stage of ripening and germination. Such induction involves, for example, the addition of compounds to the culture media of the protoplast, for example, glutamic acid and/or proline in the case of corn or alfalfa.

Preferably, a transformed cell is contacted with a compound that induces callus formation for a time and under conditions sufficient for callus formation. Alternatively, or in addition, a transgenic plant cell is contacted with a compound that induces cell de-differentiation for a time and under conditions sufficient for a cell to de-differentiate. Alternatively, or in addition, a transgenic plant cell is contacted with a compound that induces growth of an undifferentiated cell for a time and under conditions sufficient for an undifferentiated cell to grow. Compounds that induce callus formation and/or induce production of undifferentiated and/or de-differentiated cells will be apparent to the skilled artisan and include, for example, an auxin, e.g., 2,4-D, 3,6-dichloro-o-anisic acid (dicambia), 4-amino-3,5,6-thrichloropicolinic acid (picloram) or thidiazuron (TDZ). Such a medium may additionally comprise one or more compounds that facilitate callus formation/de-differentiation or growth of undifferentiated cells. For example, Mendoza and Kaeppler (In vitro Cell Dev. Biol., 38: 39-45, 2002) found that media comprising maltose rather than sucrose enhanced the formation of calli in the presence of 2,4-D.

Alternatively, or in addition, the embryonic cell is additionally contacted with myo-inositol. Studies have indicated that myo-inositol is useful for maintaining cell division in a callus (Biffen and Hanke, Biochem. J. 265: 809-814, 1990).

Similarly, casein hydrolysate appears to induce cell division in a callus and maintain callus morphogenetic responses. Accordingly, in another example, the embryonic plant cell is additionally contacted with casein hydrolysate.

Suitable culture medium and methods for inducing callus formation and/or cell de-differentiation and/or the growth of undifferentiated cells from mature embryonic plant cells are known in the art and/or described in Mendoza and Kaeppler, In vitro Cell Dev. Biol., 38: 39-45, 2002, Özgen et al., Plant Cell Reports, 18: 331-335, 1998, Patnaik and Khurana BMC Plant Biology, 3: 1-11, Zale et al., Plant Cell, Tissue and Organ Culture, 76: 277-281, 2004 and Delporte et al., Plant Cell, Tissue and Organ Culture, 80: 139-149, 2005.

Following callus induction, cell de-differentiation and/or growth of undifferentiated cells, the plant cells and/or a cell derived therefrom (e.g., a callus derived therefrom or a de-differentiated or undifferentiated cell thereof) is contacted with a compound that induces shoot formation for a time and under conditions sufficient for a shoot to develop. Suitable compounds and methods for inducing shoot formation are known in the art and/or described, for example, in Mendoza and Kaeppler, In vitro Cell Dev. Biol., 38: 39-45, 2002, Özgen et al., Plant Cell Reports, 18: 331-335, 1998, Patnaik and Khurana BMC Plant Biology, 3: 1-11, Zale et al., Plant Cell, Tissue and Organ Culture, 76: 277-281, 2004, Murashige and Skoog, Plant Physiol., 15: 473-479, 1962 or Kasha et al., (In: Gene manipulation in plant improvement II, Gustafson ed., Plenum Press, 1990). For example, a callus or an undifferentiated or de-differentiated cell is contacted with one or more plant growth regulator(s) that induces shoot formation. A suitable compound will be apparent to the skilled artisan e.g., a synthetic or natural auxin such as, for example, a compound selected from the group consisting of 2,4-dichlorophenoxyacetic acid, 3,6-dichloro-o-anisic acid, 4-amino-3,5,6-trichloropicolinic acid, indole-3-acetic acid (IAA), benzyladenine (BA), indole-butyric acid (IBA), zeatin, a-naphthaleneacetic acid (NAA), 6-benzyl aminopurine (BAP), thidiazuron, kinetin, 2iP and combinations thereof.

Suitable sources of media comprising compounds for inducing shoot formation are known in the art and include, for example, Sigma-Aldrich Pty Ltd (Sydney, Australia).

Alternatively, or in addition, the callus or an undifferentiated or de-differentiated cell is maintained in or on a medium that does not comprise a plant growth modulator for a time and under conditions sufficient to induce shoot formation and produce a plantlet.

At the time of shoot formation or following shoot formation the callus or an undifferentiated or de-differentiated cell is preferably contacted with a compound that induces root formation for a time and under conditions sufficient to initiate root growth and produce a plantlet.

Suitable compounds that induce root formation are known to the skilled artisan and include a plant growth regulator, e.g., as described supra.

Suitable methods for inducing root induction are known in the art and/or described in Mendoza and Kaeppler, In vitro Cell Dev. Biol., 38: 39-45, 2002, Özgen et al., Plant Cell Reports, 18: 331-335, 1998, Patnaik and Khurana BMC Plant Biology, 3: 1-11, Zale et al., Plant Cell, Tissue and Organ Culture, 76: 277-281, 2004, Murashige and Skoog, Plant Physiol., 15: 473-479, 1962 or Kasha et al., (In: Gene manipulation in plant improvement Gustafson ed., Plenum Press, 1990).

In an example of the invention, a callus and/or de-differentiated cell and/or undifferentiated cell is contacted with media comprising zeatin for a time and under conditions sufficient to induce shoot formation and contacted with medium comprising NAA for a time and under conditions sufficient to induce root formation. Plantlets are then grown for a period of time sufficient for root growth before being potted (e.g., in potting mix and/or sand) and being grown.

In species such as petunia and oilseed rape, adventitious shoot regeneration from bombarded leaf or petiole explants generates plastid transformants. Homoplasmic plants of soybean, carrot, and cotton are regenerated via somatic embryogenesis after bombardment of embryogenic calli, combined with the use of species-specific plastid vectors. Economically important crops such as carrot, cotton, and soybean regenerate in vitro through somatic embryogenesis and in such crops, transformation of the plastid genome is achievable through somatic embryogenesis by bombarding embryogenic non-green cells or tissues.

The generated transformed plants may be propagated by a variety of means, such as by clonal propagation or classical breeding techniques. For example, a first generation (or T1) transformed plant may be selfed to give homozygous second generation (or T2) transformant, and the T2 plants further propagated through classical breeding techniques. In this respect, the skilled artisan will be aware that the term “selfed” refers to the process of selling, which is discussed supra.

The present invention also encompasses products of repeated cycles of transformation employing plant material transformed with a promoter, active fragment or derivative of the present invention or a transgene placed operably under the control of said promoter, active fragment or derivative or a gene construct comprising said transgene operably under the control of said promoter, active fragment or derivative.

### 5. Antibodies Binding to Ch1 d Synthase Polypeptide and Fragments Thereof

The present invention also provides antibodies that bind to Ch1 d synthase and fragments thereof. The antibodies are particularly useful in determining Ch1 d expression patterns and/or expression levels in cells, tissues, organelles, organs and whole organisms, including the transformed plant cells, tissues, organelles, organs, and whole plants described herein. For example, the antibodies of the present invention may be employed to determine expression in situ of ectopically-expressed Ch1 d synthase in transformed chloroplasts and plants and correlate those expression levels and distribution with modified phenotype of the transformed organisms.

As used herein, the term “antibody” refers to intact monoclonal or polyclonal antibodies, immunoglobulin (IgA, IgD, IgG, IgM, IgE) fractions, humanized antibodies, and recombinant single chain antibodies, as well as fragments thereof, such as, for example Fab, F(ab)₂, and Fv fragments.

Antibodies are prepared by any of a variety of techniques known to those of ordinary skill in the art, and described, for example in, Harlow and Lane (In: Antibodies: A Laboratory Manual, Cold Spring Harbor Laboratory, 1988). In one such technique, an immunogen comprising the antigenic polypeptide is initially injected into any one of a wide variety of animals (e.g., mice, rats, rabbits, sheep, humans, dogs, pigs, chickens and goats). The immunogen is derived from a natural source, produced by recombinant expression means, or artificially generated, such as by chemical synthesis (e.g., BOC chemistry or FMOC chemistry). A peptide comprising any variant of the amino acid set forth in SEQ ID NO: 12 or 37 or a fragment comprising at least 5 or 6 or 7 or 8 or 9 or 10 contiguous amino acid residues of SEQ ID NO: 12 or 37 may be employed as an immunogen for antibody production.

If required e.g., for immunogens of low immunogenicity such as very short peptides, the immunogen is joined to a carrier protein, such as bovine serum albumin or keyhole limpet hemocyanin.

The immunogen and optionally a carrier for the protein is injected into the animal host, preferably according to a predetermined schedule incorporating one or more booster immunizations, and blood collected from said the animals periodically. Optionally, the immunogen is injected in the presence of an adjuvant, such as, for example Freund's complete or incomplete adjuvant, lysolecithin and dinitrophenol to enhance the subject's immune response to the immunogen. Monoclonal or polyclonal antibodies specific for the polypeptide are then purified from blood isolated from an animal by, for example, affinity chromatography using the polypeptide coupled to a suitable solid support.

Monoclonal antibodies specific for the antigenic polypeptide of interest are prepared, for example, using the technique of Kohler and Milstein, Eur. J. Immunol. 6, 511-519 (1976), and improvements thereto. Briefly, these methods involve the preparation of immortal cell lines capable of producing antibodies having the desired specificity (i.e., reactivity with the polypeptide of interest). Such cell lines are produced, for example, from spleen cells obtained from an animal immunized as described supra. The spleen cells are immortalized by, for example, fusion with a myeloma cell fusion partner, preferably one that is syngeneic with the immunized animal. A variety of fusion techniques are known in the art, for example, the spleen cells and myeloma cells are combined with a nonionic detergent or electrofused and then grown in a selective medium that supports the growth of hybrid cells, but not myeloma cells. A preferred selection technique uses HAT (hypoxanthine, aminopterin, and thymine) selection. After a sufficient time, usually about 1 to 2 weeks, colonies of hybrids are observed. Single colonies are selected and growth media in which the cells have been grown is tested for the presence of an antibody having binding activity against the polypeptide (immunogen). Hybridomas having high reactivity and specificity are preferred. Monoclonal antibodies are isolated from the supernatants of growing hybridoma colonies using methods such as, for example, affinity purification as described supra.

Various techniques are also known for enhancing antibody yield, such as injection of the hybridoma cell line into the peritoneal cavity of a suitable vertebrate host, such as a mouse. Monoclonal antibodies are then harvested from the ascites fluid or the blood of such an animal subject. Contaminants are removed from the antibodies by conventional techniques, such as chromatography, gel filtration, precipitation, and/or extraction. The Ch1 d synthase polypeptide or immunogen per se may be used in the purification process in, for example, an affinity chromatography step.

It is preferable that an immunogen used in the production of an antibody is one which is sufficiently antigenic to stimulate the production of antibodies that will bind to the immunogen and is preferably, a high titer antibody. In one example, an immunogen is an entire protein e.g., SEQ ID NO: 12 or a variant thereof as described herein. In another example, an immunogen consists of a peptide representing one or more fragments of a Ch1 d synthase polypeptide e.g., at least about 10 or 20 or 30 or 40 or 50 contiguous residues of SEQ ID NO: 12 or 37, or the full-length multi-epitope peptide represented by SEQ ID NO. Preferably an antibody raised to such an immunogen also recognizes the full-length protein from which the immunogen was derived, such as, for example, in its native state or having native conformation.

Alternatively, or in addition, an antibody raised against a peptide immunogen recognizes the full-length protein from which the immunogen was derived when the protein is denatured. By “denatured” is meant that conformational epitopes of the protein are disrupted under conditions that retain linear B cell epitopes of the protein. As will be known to a skilled artisan linear epitopes and conformational epitopes may overlap.

Alternatively, a monoclonal antibody is produced using a method such as, for example, a human B-cell hybridoma technique, e.g., Kozbar et al., Immunol. Today 4, page 72, (1983), or an EBV-hybridoma technique e.g., Cole et al. In: Monoclonal Antibodies in Cancer Therapy, Allen R. Bliss, Inc., pages 77-96 (1985), or by screening of combinatorial antibody libraries (Huse et al., Science 246, page 1275 (1989).

The further examples provided herein below illustrate the invention in more detail. These examples are provided to enable those skilled artisans to help understand and practice various aspects of the invention and therefore should not be construed as limiting. Various modifications and extensions of the invention in addition to those described herein will become apparent to those skilled artisans and therefore such modifications and extensions fall within the scope of invention.

### Example 1

### Mechanism of Chlorophyll d Biosynthesis

This example demonstrates that Ch1 d is synthesized from Ch1 a, and that the C-31 formyl-group oxygen of Ch1 d utilizes an oxygenase enzyme, such as by ¹⁸O labelling and mass spectrometry to follow chlorophyll biosynthesis in the exemplary cyanobacterium Acaryochloris marina.

### 1. Experimental Procedures

a) Culture Conditions and Growth Rate

Freeze dried 1 ml KES-sea water medium was resuspended in 1 ml H₂¹⁸O (98% ¹⁸O; Marshall Isotope, Israel) and inoculated using 100 μl of fresh Acaryochloris marina (MBIC11017) culture having an absorbance at 710 nm (Q_{y} transition) of 1.0. The cultures were incubated for 24, 45, 65, 95 and 115 hrs under continuous white light illumination having an intensity of 30 μM photons M⁻² s⁻¹. Cells were maintained in suspension by continuous shaking at about 100 rpm.

A. marina MBIC11017 culture (25 ml) having an absorption maximum at 710 nm of 0.2 was grown in ASW K⁺ES medium, in the presence of 50% ¹⁸O₂ (certified purity of 97% ¹⁸O; Cambridge Isotopes, USA), and 50% dinitrogen (N₂) gas. Aliquots of cell cultures (1 ml) were collected at 24, 48, 72, 96, 150 and 200 hrs for pigment extraction. The growth rates of the cultures were monitored by recording at the maximum absorption of culture (A₇₁₀ nm) using a spectrophotometer (Shimadzu UV-2550) having a Taylor-sphere attachment (ISR-240A, Shimadzu, Japan).

b) Pigment Extraction and HPLC Analysis/Fractionation

All pigment extractions and processing were performed quickly and under dim green light, to thereby minimize photodamage.

Briefly, A. marina cells were harvested by centrifugation 16000×g for 5 min. Pigments were extracted from cell pellets by resuspension in ice-cold 100% methanol and incubation for 15 min on ice. The pigment extracts were clarified by centrifugation, and the supernatants immediately injected into a Shimadzu VP series high performance liquid chromatographic (HPLC) system for separation on a reverse phase C18 column (Synergi Fusion 250 mm×4.6 mm; Phenomenex, Australia), at a flow rate of 1 ml/min. The C18 column was equilibrated with a 1:1 (v/v) mixture of solvent A [80% (v/v) acetonitrile; 20% (v/v) 0.2 M ammonium acetate) and solvent B (100% (v/v) methanol]. The column was developed at the same flow rate as follows:
1. 6.5 min of 1:1 (v/v) mixture of solvent A and solvent B;
2. a linear gradient from 50% (v/v) to 100% (v/v) of solvent B applied over 7 min;
3. 23 min of 100% (v/v) solvent B.

Fractions of Ch1 d and Ch1 a were collected for subsequent matrix-assisted laser desorption/ionisation-mass spectra (MALDI-MS) analysis (see below). Ch1 d and Ch1 a had retention times under these conditions of 19.7 and 21.2 min, respectively.

Ratios of pigments were calculated based on HPLC chromatogram peak areas using Shimadzu Class-VP 6.14 software (Shimadzu, Japan). Each peak was evaluated individually at its published absorption maximum wavelength and normalized to its extinction coefficient (in methanol) at that maximum wavelength, based on accepted molar extinction coefficients of Ch1 d and Ch1 d (Ch1 d, ε=77.62 mol⁻¹ cm⁻¹ at 696 nm; Ch1 a, ε=68.72 mol⁻¹ cm⁻¹ at 665 nm; Ritchie, Photosynth. Res. 89, 27-41, 2006).

c) MALDI-MS Analysis

Fractions of Ch1 a and Ch1 d were concentrated under vacuum prior to performing MALDI-MS. Concentrated pigments were mixed with a non-polar matrix, terthiopene (10 mg/ml; Sigma, USA) dissolved in acetone. One droplet (about 2-5 μl) of the mixture was dried onto a sample plate for MALDI-MS analysis. Positive ion mass spectra were obtained by performing MALDI_MS using a Voyager-DE STR (Applied BioSystems, USA) in positive ion reflector mode at 22,000 V. Readings were obtained by averaging 250 single-shot spectra, thereby providing acceptable signal-to-noise ratios. Average noise levels were treated as reading errors.

d) Calculation of Labeled Chlorophyll Isotopomer Abundance

The distributions of the measured intensities of mass peaks I₁, I₃, I₅, I₇, I₉, and I₁₁ of chlorophylls isolated from labeled cells were calculated using the following set of linear equations:

I₁=A₁*C(¹⁸O₀),

I₃=A₁*C(¹⁸O₀)+A₃*C(¹⁸O₁);

I₅=A₁*C(¹⁸O₀)+A₃*C(¹⁸O₁)+A₅*C(¹⁸O₂),

I₇=A₁*C(¹⁸O₀)+A₃*C(¹⁸O₁)+A₅*C(¹⁸O₂)+A₇*C(¹⁸O₃);

I₉=A₁*C(¹⁸O₀)+A₃*C(¹⁸O₁)+A₅*C(¹⁸O₂)+A₇*C(¹⁸O₃)+A₉*C(¹⁸O₄), and

I₁₁=A₁*C(¹⁸O₀)+A₃*C(¹⁸O₁)+A₅*C(¹⁸O₂)+A₇*C(¹⁸O₃)+A₉*C(¹⁸O₄)+A₁₁*C(¹⁸O₅);

wherein:
A₁, A₃, A₅, A₇, A₉, A₁₁ are the relative intensities of the first six ¹³C isotopomer peaks of M+0, M+1, M+2, M+3, M+4, M+5 ions of Ch1 a or Ch1 d; and
C(¹⁸O₀), C(¹⁸O₁), C(¹⁸O₂), C(¹⁸O₃), C(¹⁸O₄) and C(¹⁸O₅) are relative concentrations of the Ch1 isotopomers wherein:

C(¹⁸O₀)+(¹⁸O₁)+C(¹⁸O₂)+C(¹⁸O₃)+C(¹⁸O₄)+C(¹⁸O₅)=1.0.

For example, Ch1 a (C₅₅H₇₂N₄O₅Mg) comprises five oxygen atoms, whereas Ch1 d (C₅₄H₇₀N₄O₆Mg) comprises six oxygen atoms, such that in labeling experiments described herein which employ H₂¹⁸O or [¹⁸O]-oxygen, there will be zero (standard chlorophyll molecular weight, with ¹⁶O only), one, two, three, four, or five, ¹⁸O atoms i.e., a maximum of five ¹⁸O atoms in Ch1 a and a maximum of six ¹⁸O atoms in Ch1 d. The mass spectra of Ch1 a and Ch1 d will also comprise mixtures of ¹⁸O and ¹⁶O isotopes, which were interpreted as overlays of all individual mass spectra for Ch1 a or Ch1 d, for the following groups of isotopomers:

$$Chl a \text{:} {}_{}^{18}{O}{}_{0}^{} {}_{}^{16}{O}{}_{5}^{} , {}_{}^{18}{O}{}_{1}^{} {}_{}^{16}{O}{}_{4}^{} , {}_{}^{18}{O}{}_{2}^{} {}_{}^{16}{O}{}_{3}^{} , {}_{}^{18}{O}{}_{3}^{} {}_{}^{16}{O}{}_{2}^{} , {}_{}^{18}{O}{}_{4}^{} {}_{}^{16}{O}{}_{1}^{} , and {}_{}^{18}{O}{}_{5}^{} {}_{}^{16}{O}{}_{0}^{} ; or$$

$$Chl d \text{:} {}_{}^{18}{O}{}_{0}^{} {}_{}^{16}{O}{}_{6}^{} , {}_{}^{18}{O}{}_{1}^{} {}_{}^{16}{O}{}_{5}^{} , {}_{}^{18}{O}{}_{2}^{} {}_{}^{16}{O}{}_{4}^{} , {}_{}^{18}{O}{}_{3}^{} {}_{}^{16}{O}{}_{3}^{} , {}_{}^{18}{O}{}_{4}^{} {}_{}^{16}{O}{}_{2}^{} , {}_{}^{18}{O}{}_{5}^{} {}_{}^{16}{O}{}_{1}^{} , {}_{}^{18}{O}{}_{6}^{} {}_{}^{16}{O}{}_{0}^{}$$

$$wherein \text{:} {{\sum }_{n = 0}}^{n = 5} {[ {}_{}^{18}{O}{}_{n}^{} {}_{}^{16}{O}{}_{n}^{} ]}_{Chl a} = 1.0 , and {{\sum }_{n = 0}}^{n = 6} {[ {}_{}^{18}{O}{}_{n}^{} {}_{}^{16}{O}{}_{n}^{} ]}_{Chl d} = 1.0 .$$

Proceeding on this basis, the standard mass peak distributions were calculated for unlabeled Ch1 a (i.e., ¹⁸O₀ ¹⁶O₅) and unlabeled Ch1 d (i.e., ¹⁸O₀ ¹⁶O₆) at 1 Dalton intervals based on the abundance of the ¹³C isotope in nature, from the expected relative intensities (I) of the first six mass peaks for the M+0, M+1, M+2, M+3, M+4, M+5 ions) of Ch1 a and Ch1 d as follows:

| | M + 0 | M + 1 | M + 2 | M + 3 | M + 4 | M + 5 |
|---|---|---|---|---|---|---|
| Chl a | 100 | 75 | 42 | 15.5 | 3.1 | 0.5 |
| Chl d | 100 | 73.9 | 41.3 | 15.1 | 3.7 | 0.4 |

A difference in mass of two Daltons was expected from each incorporation of ¹⁸O in the newly synthesized chlorophylls.

### 2. Experimental Results

The carboxyl oxygen atoms at C-17 of Ch1 a and Ch1 d are derived from water, and the C-13 oxo group in ring E of Ch1 a is formed though an oxygenase mechanism in most cyanobacteria, algae and plants e.g., Porra et al., Eur. J. Biochem. 239, 85-92 (1996). To examine the origins of oxygen atoms in Ch1 d, the ¹⁸O labelling experiments described herein were conducted, employing H₂¹⁸O in culture medium or ¹⁸O₂ in the gaseous phase.

a) Ch1 d Synthesis in the Presence of ¹⁸O Isotope

Table 2 provides the ratio Ch1 a to Ch1 d, and the percentage of Ch1 d in cultures, as determined from labelling of cultures with H₂¹⁸O in culture medium or ¹⁸O₂ in the gaseous phase, after 120 hr culture time. Data are presented as the mean value for n=7 samples, and the errors were calculated by STDEV. The data indicate that, notwithstanding slightly higher mean incorporation of the isotope from liquid than gas, no statistically significant difference between the two culture conditions was observed. Significant levels of Ch1 d were produced in the presence of the isotope.

TABLE 2
Chlorophyll ratio and percentage Chl d in A. marina cultures

| Isotope used | Ratio Chl a to Chl d | Chl d (%) |
|---|---|---|
| H₂¹⁸O | 0.0616 ± 0.018 | 94.19 ± 1.60 |
| ¹⁸O₂ | 0.0594 ± 0.008 | 94.39 ± 0.69 |

b) Time Courses of Chlorophyll Syntheses in Media Comprising Labeled Water

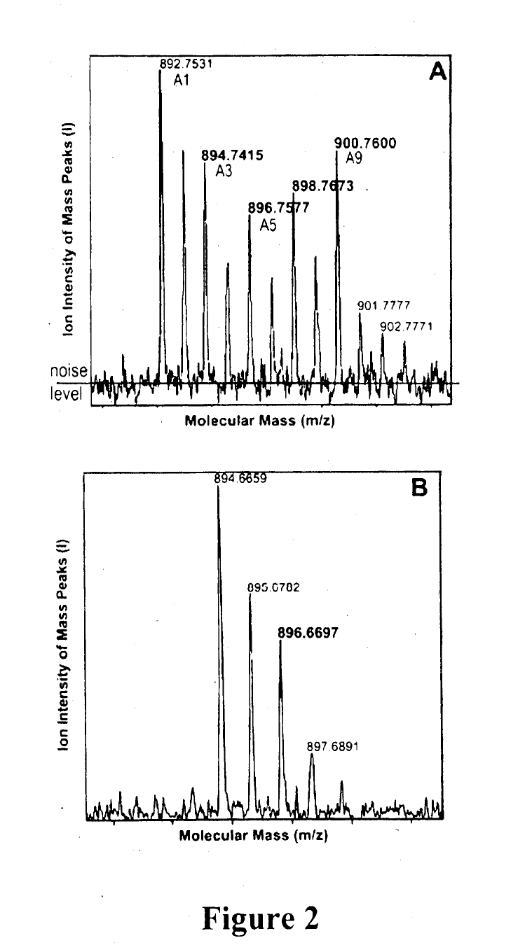

The maximum mass (m/z) of newly synthesised Ch1 a in the presence of ¹⁸O isotope under the conditions employed herein was 900.76 Da. This mass is about 8 Da heavier than the mass of unlabeled Ch1 a (m/z=892.5), indicating that at four ¹⁸O atoms were incorporated into newly-synthesized Ch1 a under these conditions, derived from labeled water (FIG. 2a).

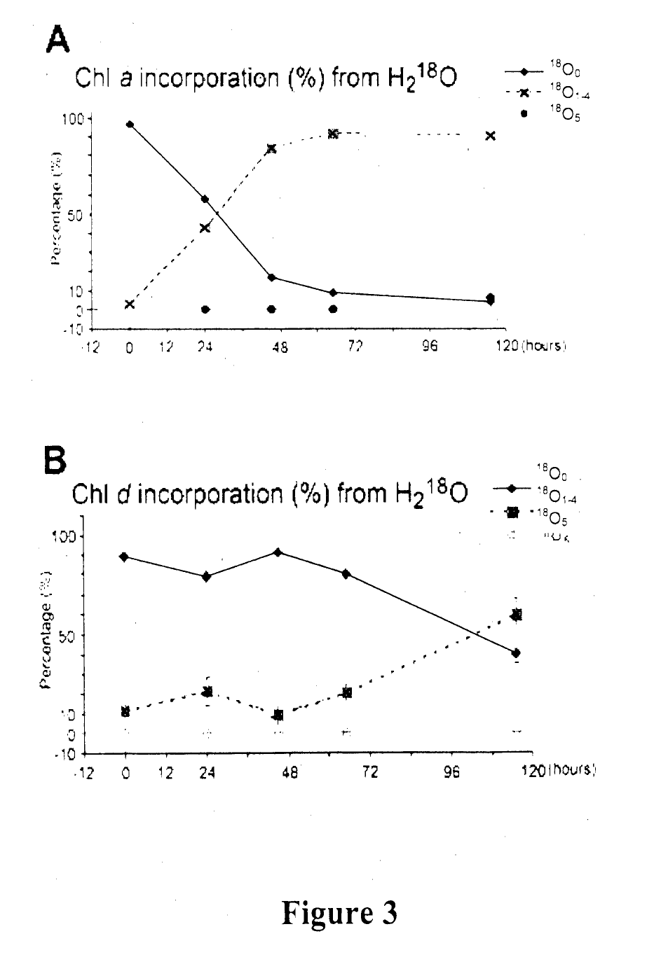

The percentage of standard mass Ch1 a decreased during the time cells were cultured in the presence of H₂¹⁸O, and less than 10% of ¹⁸O₀-Ch1 a was observed in the Ch1 a sample extracted after 65 hr, (FIG. 3a). The data in FIGS. 2a and 3a indicate collectively that 42% of each Ch1 a molecule extracted after 24 hr culture in media comprising H₂¹⁸O comprised up to four ¹⁸O atoms, and that nearly 90% of Ch1 a molecules comprised at least one ¹⁸O atom after 45 hr. Accordingly, almost all extracted Ch1 a after 45 hr was newly-synthesized, suggesting that Ch1 a is turned over within 45 hr in A. marina cultured under these conditions. A trace amount of Ch1 a having ¹⁸O atoms occupying all five positions in the molecule was observed after 115 hr incubation i.e., at about 5 days, may be due to utilization of ¹⁸O₂ from labeled water by photosynthesis.

In contrast to Ch1 a, the mass spectrum of the extracted Ch1 d provided a similar mass distribution for Ch1 d obtained from [¹⁸O₂]-labeled cultures as for standard Ch1 d i.e., m/z=894.5, after growth for 48 hr (FIG. 2b). A slight change in the percentage of standard mass Ch1 d was observed after 24 hr, however no corresponding increase in ¹⁸O₁-Ch1 d was detected, Ch1 d molecules having more than two ¹⁸O atoms incorporated were only observed in samples extracted after 115 hr culture time (FIG. 3b).

Comparison of the time-course profiles for Ch1 a (FIG. 3a) and Ch1 d (FIG. 3b) suggests that Ch1 a is synthesized before Ch1 d. As shown in FIGS. 3a and 3b, 50% of newly-synthesized Ch1 a is detectable at 24 hr, whereas newly-synthesized Ch1 d is detectable only after about 65 hr. These data, and the rapid turnover of Ch1 a, suggest Ch1 d is synthesized from Ch1 a.

c) Time Courses of Chlorophyll Syntheses in Labeled Oxygen Gas

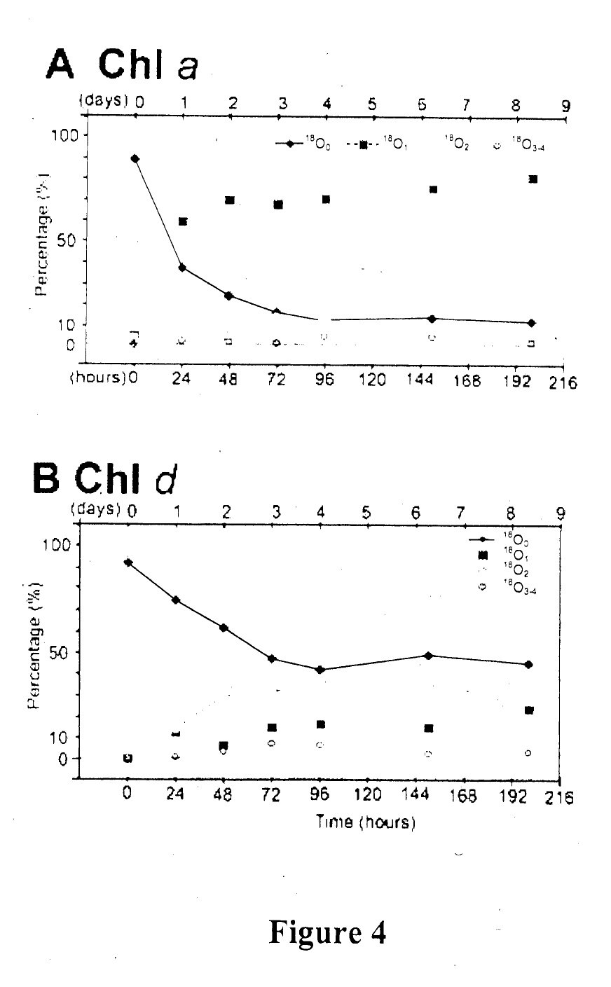

Data in FIG. 4a indicate that about 40% of Ch1 a molecules isolated from A. marina cells that were grown in an atmosphere comprising ¹⁸O₂ had a standard mass of 892.44 Da after 24 hr. During the same time period, about 60% of Ch1 a molecules comprised a single ¹⁸O atom, as determined by a mass of 894.44 Da (FIG. 4a). The incorporation of a single ¹⁸O atom from labeled oxygen gas is consistent with the derivation of the oxygen atom at the C-13¹ position in the E-ring of Ch1 a from ¹⁸O₂, and also suggests that other oxygen atoms in Ch1 a are from another source.

The time-course of incorporation of ¹⁸O₂ from labeled oxygen gas suggested a turnover of Ch1 a consistent with data obtained using labeled water. Accordingly, almost all Ch1 a molecules comprised an ¹⁸O atom after 48 hr irrespective of the source of labeled oxygen.

The data presented in FIG. 4b indicate that two ¹⁸O atoms were incorporated into each molecule of Ch1 d up to about 72 hr incubation time. The increase in labeled Ch1 d in the presence of labeled gas was also accompanied by a decrease in the amount of unlabeled Ch1 d (FIG. 4b) up to about 72 hr. One ¹⁸O atom was detected in Ch1 d at 24 hr before dropping at 48 hr (FIG. 4b). This labelling followed inversely the Ch1 a labeling pattern and suggests that the unlabeled Ch1 a was the precursor of Ch1 d. There was also a gradual increase in the incorporation of singly-labeled Ch1 d up to 205 hr at which time the amount of doubly-labeled Ch1 d began to fall, possibly because the labeled oxygen gas was being diluted with unlabeled oxygen from photosynthesis after longer incubation periods.

### 3. Summary

The data provided in this example support the conclusion that the four carboxyl oxygen atoms at the C-13³ and C-17³ positions of both Ch1 a and Ch1 d are derivable from water, whereas the oxygen atom at the C-13¹ position in both Ch1 a and Ch1 d is derivable from ¹⁸O₂, most likely involving an oxygenase mechanism.

The data herein also support the conclusion that Ch1 a is a biosynthetic precursor of Ch1 d, and that the formyl oxygen at C-3 of Ch1 d is derivable from ¹⁸O₂, most likely involving a Ch1 d synthase that belongs to the class of oxygenase enzymes.

### Example 2

### Substrate Requirements of Ch1 d Synthase

This example provides evidence that chlorophyll d synthase is a dioxygenase distinct form CAO-type enzymes.

### 1. Experimental Procedures

a) Culture Conditions and Growth Rate

A. marina cells were cultured under white light at 22° C. with gasification by bubbling air, or air mixed with gaseous carbon monoxide [about 1:1 (v/v)], or nitrogen gas. Chlamydomonas reinhardtii cells were cultured under the same conditions as a control.

a) Pigment Extractions and Analyses

A long needle-syringe was used to sample cells, and pigments were extracted from cells as described herein above using methanol, HPLC analyses were also as described above, except that the Agilent 1100 series HPLC system was employed with the reverse phase C18 column being equilibrated and run with 100% methanol solvent.

### 2. Experimental Results

In contrast to A. marina, cells of C. reinhardtii comprise Ch1 a and Ch1 b. It is known that formyl group at the C-7 position of Ch1 b is derived from the C-17 methyl group of Ch1 a by a dioxygenase reaction catalyzed by chlorophyll a oxygenase.

To determine whether or not chlorophyll d synthase is different to chlorophyll a oxygenase, and whether or not chlorophyll d synthase and chlorophyll a oxygenase belong to different enzyme classes, the effects of gasification with air, air/CO mixture and nitrogen on pigment production by A. marina and C. reinhardtii were measured.

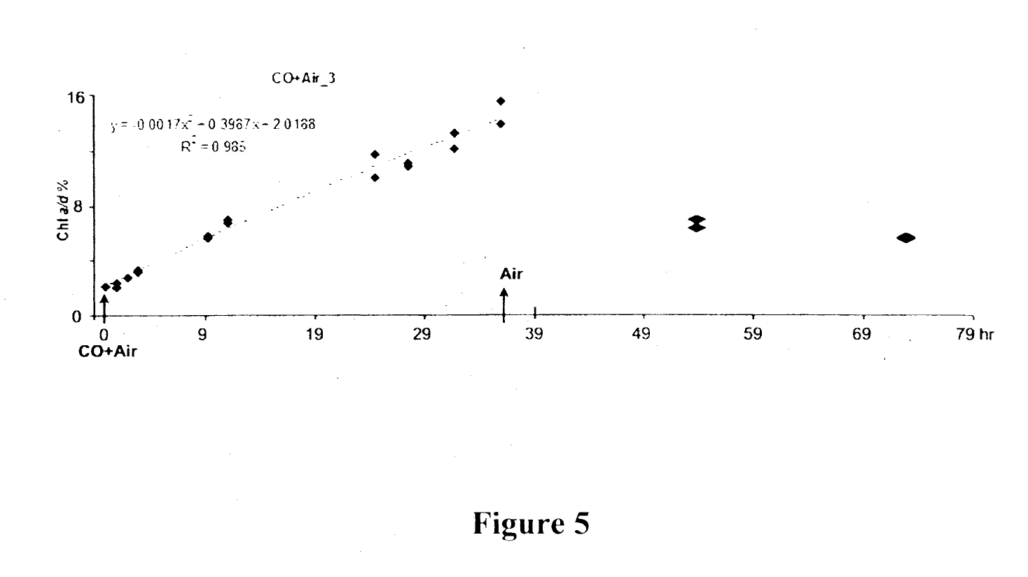

Data presented in FIG. 5 show that the ratio of Ch1 a/Ch1 d in A. marina increased from 2% to about 15% after 36 hr incubation under a mixture of air and carbon monoxide, suggesting that the concentration of Ch1 d decreased under these conditions.

The reduced ratio of Ch1 a/Ch1 d in A. marina observed following incubation under a mixture of air and carbon monoxide was rapidly reversed, and a ratio of Ch1 a/Ch1 d close to normal

Was reached within 20 hr when the carbon monoxide in the air line was replaced by fresh air (FIG. 5).

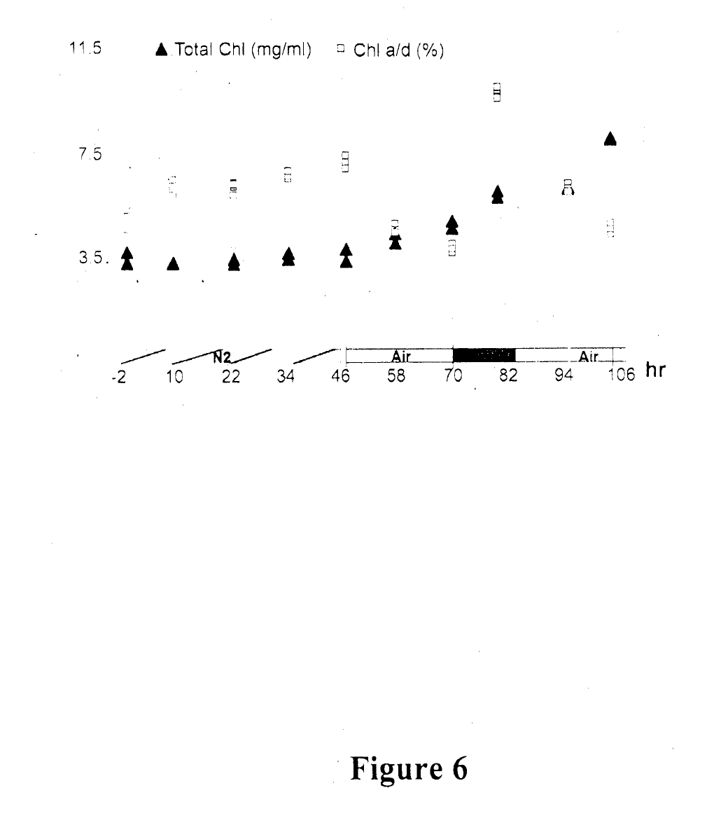

Data presented in FIG. 6 show that the ratio of Ch1 a/Ch1 d in A. marina was not affected significantly over a 46 hr incubation period under pure nitrogen atmosphere, however the total amount of chlorophyll produced increased when the nitrogen was replaced with air. Although the ratio of Ch1 a/Ch1 d dropped at the point of replacing a nitrogen atmosphere with fresh air, this was most likely due to increased levels of Ch1 d because total chlorophyll content was increasing in the same period.

In contrast to A. marina, no significant changes in the ratio of Ch1 a/Ch1 b were detected in C. reinhardtii grown under a mixture of air and carbon monoxide (data not shown).

### 3. Summary

Data presented in FIGS. 5 and 6 suggest that Ch1 d synthase is a different type of enzyme from CAO, although both of them use dioxygen as substrate of oxidative reaction. Rapid inhibition of Ch1 d biosynthesis by carbon monoxide, as determined by an increase in the ratio of Ch1 a/Ch1 d, is consistent with the Ch1 d synthase enzyme being a member of the cytochrome P450 oxygenase family of proteins, and that carbon monoxide is a potent inhibitor of the enzyme. This inhibition also appears to be reversible.

This is the first evidence indicating that Ch1 d synthase enzyme is a cytochrome P450 oxygenase, and that the enzyme is different from chlorophyllide a oxygenase (CAO), and divinyl chlorophyllide reductase, or a radical SAM enzyme).

### Example 3

### Isolation of Gene Encoding A. marina Ch1 d Synthase

This example provides an isolated nucleic acid encoding Ch1 d synthase of a cyanobacterium, A. marina.

### 1. Experimental Methods

a) Isolation of Nucleic Acids Encoding Cytochrome P450 Family Proteins

From the genome sequence of A. marina provided by Swingley et al., Proc. Natl. Acad. Sci. (USA) 105, 2005-2010 (2008), eleven gene loci were selected that encode putative cytochrome P450 oxygenase enzymes. Of these eleven gene loci, six were not present in the Salton Sea strain of A. marina, as determined by DNA hybridization, and were discarded on the basis that Ch1 d synthase is ubiquitous in Ch1 d-producing strains of A. marina. Thus, five gene loci were identified as putative Ch1 d synthase-encoding genes from this analysis: AMI_{—}0606; AMI_{—}0824; AMI_{—}3563; AMI_{—}4161; and AMI_{—}5780. The sequence at gene locus AMI_{—}3563 is not closely related to the sequences of cytochrome P450 oxygenases from other cyanobacteria.

Total RNA was isolated from A. marina cells grown under a mixture of air and carbon monoxide, using TRI reagent (Ambion, USA), and purified using a DNA-free kit (Ambion, USA) according to the manufacturer's instructions. RNA concentrations were measured using a NanoDrop spectrophotometer (Eppendorf). Then, cDNA was synthesized from 1 μg RNA using Script II kit (Invitrogen, USA) according to the manufacturer's instructions.

Real time PCR was performed using a Power SYBR Green PCR Master Mix (ABI) on ABI real time thermal cycler, according to the manufacturer's instruction. The real time PCR cycling condition was: 50° C. for 2 min; 95° C. for 2 min; and 40 cycles, each consisting of 95° C. for 15 sec then 60° C. for 45 sec then 72° C. for 45 sec. Melting curve analysis was performed to confirm the purity of the qPCR products. All samples were performed in duplicate and replicate data were collected for further comparative analysis. Relative quantification analysis for RT-PCR was performed using the Relative Expression Software Tool (REST-MCS).

PCR primers for each of the five candidate genes were as follows:

| Locus | Forward primer sequence | Reverse primer sequence |
|---|---|---|
| AM1_0606 | ATGAGCGATTTCAGCAGCTCAGT | GCAAGGAGCAGGCTGAGAATATCTTGT |
| | (SEQ ID NO: 1) | (SEQ ID NO: 2) |
| AM1_0824 | TTAGCCACTGCTGACCTCAATGA | TGGCTGAAAGGCTTGAGGATTCG |
| | (SEQ ID NO: 3) | (SEQ ID NO: 4) |
| AM1_3563 | TTGGCAGGTGCTAGGAGAACAG | AAGGATGACTATCTGTCAGGATGG |
| | (SEQ ID NO: 5) | (SEQ ID NO: 6) |
| AM1_4161 | CTGTGGTGTTAGACGGTGTGCTCTAT | AAACTGGCGAAGCAGATGGCTG |
| | (SEQ ID NO: 7) | (SEQ ID NO: 8) |
| AM1_5780 | ACAGCCTTGACTTGGGCAATGTAC | ATCGAGCTGATAGCCACATAGCTCC |
| | (SEQ ID NO: 9) | (SEQ ID NO: 10) |

b) Cloning and Expression of Nucleic Acids Encoding Cytochrome P450 Proteins

The amplified gene fragments were employed to isolate full-length cDNAs which were then cloned into a bacterial plasmid in operable connection with a promoter for expression in E. coli, and E. coli cells were then transformed with the plasmids according to standard protocols. For transformation, competent bacterial cells were thawed on ice, and 3 μl recombinant plasmid DNA was added and the cells incubated on ice for 5 min. Cells were heat-shocked by incubation at 42° C. for 30 sec, and returned to ice for a further 2 min. LB media (200 μl) was added to the cells, and the mixtures shaken at 37° C. for 30 min to 1 hr. The transformed cells were plated onto agar plates comprising appropriate selective antibiotics and grown overnight at 37° C.

To express the encoded A. marina proteins in transformed E. coli, scrapings of 10-20 colonies inoculated into 3 ml LB medium comprising selective antibiotic, and the cells were cultured for about 1.5 hr at 37° C. with constant agitation at about 200 rpm to keep cells in suspension. The optical density of the cell cultures was in the range of OD₆₀₀=0.8 to OD₆₀₀=1 following this culture protocol. Protein expression was induced by addition of 1 mM. IPTG and about 5 μl Haemin (<1 μM) to cell cultures, and incubation for 3 hr at 37° C., or for 5 hr at 25° C.

Standard SDS PAGE was performed to confirm expression of recombinant protein. Briefly, cells were collected by centrifugation at 5000×g for 10 mins, and resuspended in 30 ml of buffer comprising 50 mM Tris, 300 mM NaCl, 10 mM imidazole, at pH 8, and frozen at minus 80° C. Cells were thawed and lysed using a french press at 12,000 p.s.i. 2-3 times, and the membranes collected by centrifuged at 15,000×g for 15 mins The soluble supernatants and the cell pellets were resuspended separately in denaturing buffer comprising 8 mM urea and 2 mM DTT.

c) Ch1 d Synthesis by Recombinant E. coli Expressing Ch1 d Synthase Gene

Chlorophyll a that had been extracted from spinach leaves, dried and frozen, was dissolved in ethanol (200 μl) and sufficient Ch1 a to turn cultures green was added to E. coli cells expressing cytochrome P450 oxygenases AMI_{—}3563 or AMI_{—}4161, or one of the three Rieske Fe—S centre domain proteins, AMI_{—}1961, AMI_{—}2850 or AMI_{—}4158. The reaction mixtures were incubated at room temperature in the dark for a time sufficient to produce Ch1 d i.e., about 30 min, and the cells collected by centrifugation. The media were removed and discarded, and cells re-suspended in 25 μl acetone and 975 μl ethanol mixture.

To confirm Ch1 d synthesis, fluorescence emission was determined between 650 nm and 750 nm, following excitation at 440 nm (Ch1 a), for each sample.

### 2. Experimental Results

a) Transcriptional Regulation of Expression by Carbon Monoxide and Oxygen

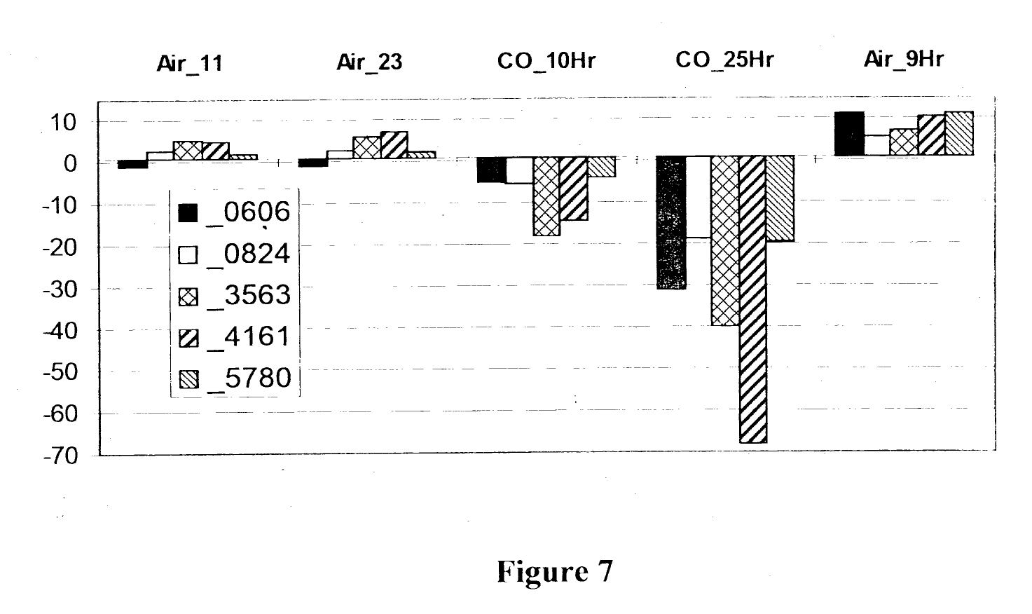

Transcription in E. coli of most of the five cloned A. marina cytochrome P450 oxygenase-encoding genes was enhanced in cells incubated under air for 11 hr or 23 hr, and reduced when carbon monoxide was bubbled into cultures (FIG. 7). The notable exception to this trend was AMI_{—}0606, the expression of which was reduced under both air and carbon monoxide (FIG. 7). Transcription of AMI_{—}3563 and AMI_{—}4161 was more responsive to gas environment than the other genes tested. When the cultures were returned to air after growth for 25 hr under carbon monoxide, (Air_{—}9 hr, FIG. 7), expression of all five clones was enhanced.

b) Ectopic Expression of Recombinant Proteins

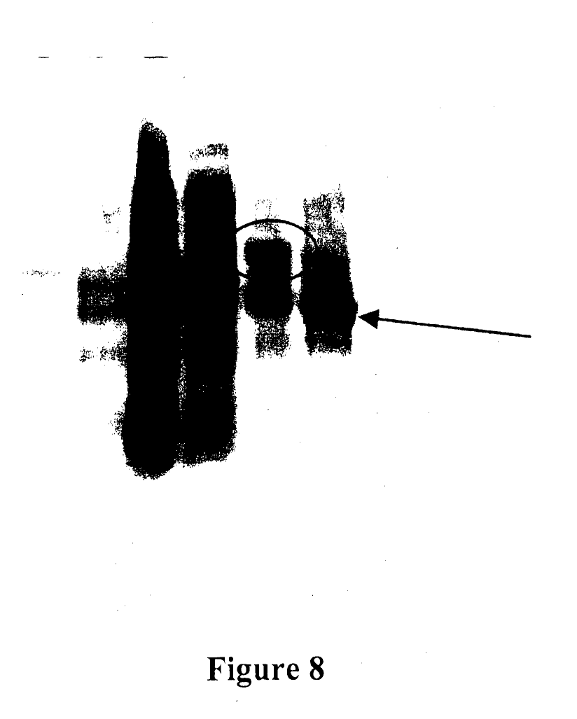

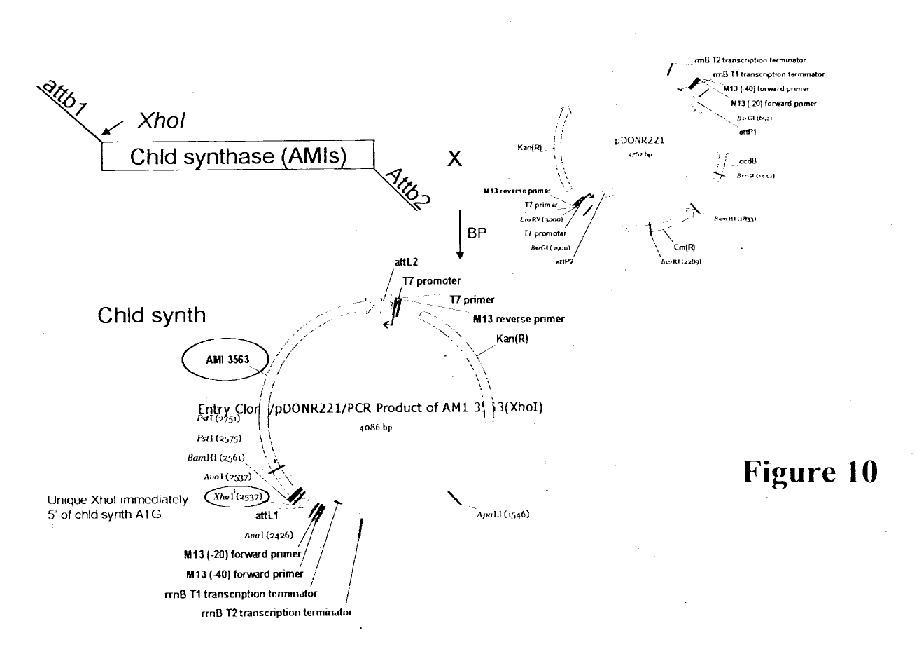

By way of example, FIG. 8 indicates proteins expressed by E. coli comprising a recombinant plasmid that contains the A. marina cytochrome P450 oxygenase-encoding nucleic acid designated AMI_{—}3563. The molecular weight of recombinant protein encoded by AMI_{—}3563 is 58 kDa (circled in FIG. 10). Data indicate that the protein is present in the membrane fraction of the cells.

b) Ectopic Expression of Recombinant Proteins

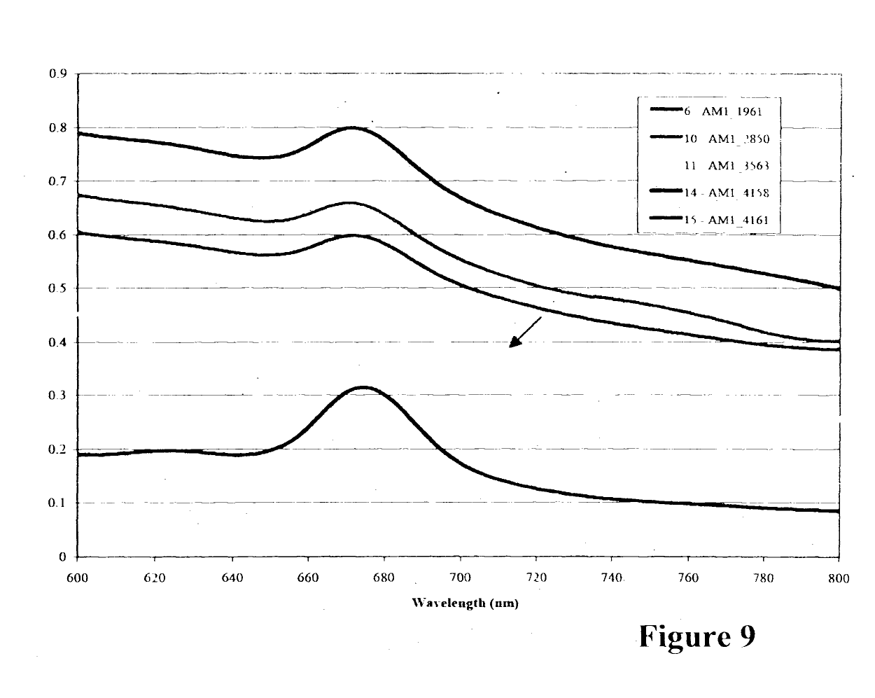

The candidate Ch1 d synthase genes were expressed in E. coli in the presence of Ch1 a and fluorescence emission determined at between 650 nm and 750 nm. Data presented in FIG. 9 indicate the presence of an extra red shift peak for cultures expressing AMI_{—}3563, with maximum at about 710 nm, characteristic of Ch1 d. HPLC analysis confirms that E. coli expressing AMI_{—}3563 had synthesized Ch1 d in this reaction mixture.

### 3. Summary

The data provided herein confirm that the AMI_{—}3563 locus encodes a Ch1 d synthase of the cytochrome P450 oxygenase family of proteins, that is capable of converting Ch1 a into Ch1 d at room temperature in the dark. The data herein also indicate that the Ch1 d synthase of A. marina is functional across different kingdoms.

The nucleotide sequence of this Ch1 d synthase-encoding gene is presented in SEQ ID NO: 11, and the amino acid sequence of A. marina Ch1 d synthase is presented in SEQ ID NO: 12. The nucleotide sequence of a fragment of the full-length open reading frame of the A. marina. Ch1 d synthase-encoding gene which is amplified using primers comprising the sequences of SEQ ID NO: 5 and SEQ ID NO: 6 is set forth in SEQ ID NO: 13. Primers for amplifying the full-length open reading frame of the A. marina Ch1 d synthase-encoding gene comprise sequences set forth in SEQ ID NO: 14 and SEQ ID NO: 15.

### Example 4

### Isolation of Variants of A. marina Ch1 d Synthase Genes

This example provides variants of the isolated nucleic acid encoding Ch1 d synthase of A. marina set forth in SEQ ID NO: 11 and variants of the encoded polypeptide set forth in SEQ ID NO: 12, supporting the premise that the genus of A. marina Ch1 d synthase-encoding genes is circumscribed by a minimum percentage identity of at least 85% identity to SEQ ID NO: 11.

### 1 Experimental Methods

a) A. marina Isolates

Isolates of the cyanobacterium Acaryochloris marina were obtained from Stromatolite, Shark Bay, Western Australia, Australia, and from coral sediments of Heron Island, Queensland, Australia, and from Sydney River, New South Wales, Australia.

b) Nucleic Acid Amplification and Purification

For amplifications, cells were harvested from 1 ml cell culture using microcentrifuge at 13,200 rpm for 2 min. The cell pellets were each washed in water twice and resuspended in 100 μl nuclease-free water, and incubated at 100° C. by immersion of tubes in a boiling water bath for 3 minutes to release DNA from cells. Reactions (20 μl each) comprised 4 μl of a 5× Mango Taq Polymerase Reaction buffer, 0.2 mM dNTP mixture, 1.25 U Mango Taq Polymerase (Bioline, USA), about 20 ng DNA template, 0.25 μM forward primer and 0.25 μM reverse primer. Amplification reactions (PCR) were performed using the following primer pairs:

| | |
|---|---|
| 1. | AM1_3563 fusion primer1 (forward; SEQ ID NO: |
| | 18): |
| | 5′-cgcgcgccagccatATGACAGGTCTATCAGCCGGA-3′; |
| and | |
| 2. | AM1_3563 fusion primer2 (reverse; SEQ ID NO: |
| | 20): |
| | 5′-gttagcagccggatccTTATGACCGCAAGATAAATCGAG-3′; |
| or | |
| 3. | AM1_3563_231F (forward; SEQ ID NO: 19): |
| | 5′-GGGAAACCTATTTGGCAGGT-3′; |
| and | |
| 4. | AM1_3563 fusion primer2 (reverse; SEQ ID NO: |
| | 20): |
| | 5′-gttagcagccggatccTTATGACCGCAAGATAAATCGAG-3′. |

Nucleotide residues underlined and italicised supra are homologous or complementary to sequence of the vector pET15B.

As a negative control for the amplification reactions, template DNA was omitted. Genomic DNA of Acaryochloris marina MBIC11017 was used as a positive control.

Amplification cycles were; 94° C. for 2 min, followed by 30 cycles of 94° C. for 10 sec, 52° C. for 15 sec and extending at 72° C. for 2 min. The final extension was set at 72° C. for 5 min after 30 cycles.

The amplification products were resolved by electrophoresis on 1.5% (w/v) agarose gels, and excised from the gel for further purification. Purified DNA fragments were sequenced at Australian Genome Research Facility Ltd (AGRF) with the appropriate primers.

b) Nucleic Acid Sequence Information

Acaryochloris marina CCMEE 5410 genomic sequence information was obtained from Prof. Blankenship, Washington University, St Louis, USA.

c) Pigment Analyses

Cells of A. marina isolates were harvested by centrifugation and resuspended in 200 μl pre-chilled methanol per 2 mL cell culture. The extracted pigments were subjected to High Performance Liquid Chromatography (HPLC) analysis. All work was performed under dim green light illumination.

HPLC analysis was conducted on a Shimadzu VP series HPLC system using a reversed-phase C18 column (Synergi Fusion-RP 80A, 250 mm×4.6 mm, Phenomenex). The column was equilibrated using solvent A (85% methanol and 15% 50 mM ammonium acetate). HPLC program was: water-methanol gradient at flow rate of 1 ml/min, 0 to 10 min linear gradient 85% (solvent A) to 100% methanol (solvent B), 10 to 30 min 100% methanol, Ch1 a was separated from Ch1 d with 2-3 min difference in their retention time. Pigment profiles were detected by an attached photodiode array detector (SPD-M10Avp, Shimadzu). The ratio of each pigment was calculated based on HPLC chromatogram peak area at their extinction coefficient reading wavelength. The Molar extinction coefficient (ε) of different pigment in methanol was listed as follows:

ε=77.62 mol⁻¹ cm⁻¹ at 696 nm for Ch1 d;
ε=68.72 mol⁻¹ cm⁻¹ at 665 nm for Ch1 a;
ε=44.6 mol⁻¹ cm⁻¹ at 667 nm for pheophytin a;
ε=133 mol⁻¹ cm⁻¹ at 452 nm for zeaxanthin; and
ε=145 mol⁻¹ cm⁻¹ at 448 nm for α-carotene.

### 1. Results

All A. marina isolates contained Ch1 d as their major photopigment. HPLC analysis showed Ch1 d concentrations from 41% to 55% in total pigment, and that the Sydney River isolate contains the highest level of zeaxanthin (23%), that the Heron island (CRS) isolate contains the highest level of α-carotene (39%) (Table 3). All strains were shown to have only trace amounts of Ch1 a, less than 2% in each case.

TABLE 3
HPLC pigment analyses of geographical isolates of A. marina

| | Shark Bay | Sydney | Heron Island |
|---|---|---|---|
| Relative ratio (%) | (stromatolite) | River | (CRS) |
| Chl d in total pigment (%) | 54.5% | 41.4% | 42.8% |
| Chl a in total pigments (%) | 1.2% | 0.8% | 0.5% |
| Zeaxanthin in total (%) | 15.2% | 22.9% | 17.7% |
| α-carotene in total (%) | 28.6% | 34.7% | 38.7% |
| Pheophytin a in total (%) | 0.4% | 0.2% | 0.3% |
| Chl d/Chl a ratio | 45.42% | 51.75 | 85.6% |
| Chl d/total Chls % | 97.85% | 98.10% | 98.85% |

Total pigment shown in Table 3 includes chlorophylls and carotenoids, and total chlorophylls are combined values for Ch1 a and Ch1 d. All calculations were based on peak area using Shimadzu Class-VP 6.14 software at their absorption maxima wavelength (extinction coefficient wavelengths).

The AMI 3563 fusion primers (SEQ ID Nos: 18 and 20) are designed to cover the full length sequence of SEQ ID NO: 11 (1533 bp), from the start codon (ATG) to the translation stop codon (TAA). These primers successfully isolated nucleic acids from the genomic DNAs of geographical isolates of A. marina at Sydney River, New South Wales, Australia (SEQ ID NO: 21) and in coral sediments of Heron Island, Queensland, Australia (CRS; SEQ ID NO; 23). The sequenced amplification products showed very high sequence identity to SEQ ID NO: 11 (Table 4). However, using these primers, the inventors were not able to routinely isolate DNA from the Shark Bay isolate. The inventors did obtain an amplification product from the Shark Bay isolate using primers derived from the conserved regions of SEQ ID NO: 11, with primer AMI_{—}3563_{—}231F (SEQ ID NO: 19) and AMI_{—}3563 fusion primer2 (SEQ ID NO: 20). The Ch1 id encoding-encoding sequence of the Shark Bay isolate (SEQ ID NO: 25) was incomplete, however was 85% identical in sequence over the region of sequence conservation with SEQ ID NO: 11. The details of divergence in Ch1 d synthase are demonstrated in the alignment of genes and translated amino acid sequences were elucidated by sequence alignments using the Clustal W program.

Using the sequence set forth in SEQ ID NO: 11 to search the Acaryochloris marina CCMEE5410 genome sequence database (unpublished data), only one sequence was identified (SEQ ID NO: 27). The genomic sequence has 98% sequence identity overall to the AMI-3563 sequence of Acaryochloris marina MBIC11017 (SEQ ID NO: 11).

TABLE 4
Chl d synthase ORFs from geographical isolates of A. marina

| Isolate | ORF length | Identity to SEQ ID NO: 11 |
|---|---|---|
| BM1C11017 (AM1_3563) | 1533 bp | 100% |
| CCMEE5410 (genomic) | 1533 bp | 98% |
| Shark Bay (Stromatolite) | 1326 bp | 85% |
| Heron Island (CRS) | 1533 bp | 98% |
| Sydney River | 1524 bp | 100% |

### Example 5

### Expression Vectors Comprising A. marina Ch1 d Synthase Genes

This example provides gene constructs for expressing isolated nucleic acid encoding Ch1 d synthase of A. marina in Escherichia coli, cyanobacteria and high plants.

a) Escherichia coli Expression System

The AMI_{—}3563 gene was amplified by PCR with AMI_{—}13563 fusion primer1 (SEQ ID NO: 18) and AMI_{—}3563 fusion primer2 (SEQ ID NO: 20) from Acaryochloris marina genomic DNA. The fusion primers contain 15 bases (red letters) of homology at each end to the pET15B vector, to facilitate sub-cloning of the amplification product into that vector for expression purposes. The genomic sequence is set forth in SEQ ID NO: 27, and the encoded polypeptide sequence is set forth in SEQ ID NO: 28 hereof. The amplification product is inserted into pET15B vector digested with BamHI and NdeI restriction enzymes. The ligation reaction is performed under standard conditions using combined insert gene fragments and digested pET15B vector at a 2:1 (w/w) molar ratio in a final volume to 10 μl. The ligated plasmid is used to transform E. coli blue competent cells according to standard procedures.

A minimum 6 positive colonies were picked with toothpick and plated on antibiotic selective LB plates for overnight at 37° C. To screen the positive clones for DNA inserted in the correct orientation, PCR was performed by using T7 forward primer and AMI_{—}3563 fusion primer2 (reverse), or T7 reverse primer and AMI_{—}3563 fusion primer1. Plasmid having the Ch1 d synthase-encoding gene in the correct orientation was isolated using the Plasmid Miniprep system (Promega), then transformed to competent cells [BL21(DE3) pLysS or BL21(DE3) Rosetta] for further analysis.

For target protein expression, The E. coli cells are grown to OD600 of 0.5, then induced by adding 1 mM IPTG, mM NADPH for 6-24 hours.

To test protein activity, different concentrations of chlorophyll d precursors (chlorophyll a or chlorophyllide a) are added to the cultured cells, and the cells are incubated in the dark for 6-24 hours. The cells are then washed and resuspended in buffer and spectra recorded in vivo (in cells in PBS buffer) and/or in vitro such as by pigment extraction using butanol or methanol.

b) Synechocystis PCC 6803 Expression Systems

The A. marina Ch1 d synthase-encoding open reading frame (SEQ ID NO: 11, 21, 23, 25, or 27) is amplified by PCR using the following specific primers:

| | |
|---|---|
| 1. | AM1_3563R1_Fwd: |

(SEQ ID NO: 29)

| | |
|---|---|
| | 5′-GGAGAACCACATTAAAATGACAGGTCTATCAGCCGGA-3′; |
| and | |
| 2. | AM1_3563 Rev: |

(SEQ ID NO: 30)

5′-GCTGACTCATACCAGGTTATGACCGCAAGATAAATCGAG-3′.

A second round of PCR on the first round PCR product is performed with the same reverse primer (AMI_{—}3563 Rev; SEQ ID NO: 30) and the following forward primer: AMI_{—}3563R2_Fwd:

(SEQ 1D NO: 31)
5′-ATGGGGCGATTCAGGAGAACCACATTAAAATG-3;.

The underlined sequences in the above-named primers comprise a Shine-Dalgarno sequence positioned before the start codon (ATG) of the Ch1 d synthase target gene, for ribosome binding and for optimal translation by Synechocystis. Other known ribosome binding sites may be employed.

The second round PCR product is inserted directly into the StuI site 5′-of the kanamycin-resistance gene cassette (SEQ ID NO: 32) of the pBluescript plasmid designated pBS-HO2-Kan (Associate Professor Robert Willows, Macquarie University, Sydney, Australia). The StuI cloning site is very close to the stop codon of the kanamycin resistance gene, such that the Ch1 d synthase target gene is translated as part of an operon in kanamycin-resistant cells. The kanamycin-resistance gene is inserted into the HpaI site of the Synechocystis s11875 gene encoding heme-oxygenase 2. The heme-oxygenase 2 gene is non-essential for Synechocystis and is disrupted by the kanamycin cassette to impart kanamycin resistance to Synechocystis cells without adversely affecting cell viability.

Synechocystis PCC 6803 is cultured in BG11 fresh water medium (with and without 5 mM Glucose) to reach OD730 of 0.5 (in the mid-log growth phase). Cells are harvested by centrifugation and resuspended in fresh medium to OD730 of 2,5. The transformed cells are plated on solid BG11 medium (with 5 mM Glucose) plates for overnight, then transferred to plates comprising 15-25 μg/ml kanamycin and/or other antibiotic as required. The cells are cultured for 7-14 days until colonies become visible.

Pigments of transformed cells are analyzed in vivo and in vitro by spectrophotometer. Further photo-physiological parameters analysis is performed following standard analysis methods.

c) Plant Expression System

The Ch1 d synthase target gene is extracted from A. marina using the amplification primers in Table 5 in standard PCR.

TABLE 5
Amplification primers for isolation of A. marina Chl d synthase genes
to sub-clone into plant expression vectors

| Primer (SEQ ID NO:) | Sequence |
|---|---|
| AM1_3563AttB_FW | ggggacaagtttgtacaaaaaagcaggcttaATGACAGGTCTAT |
| (SEQ ID NO: 33) | CAGCCGG |
| AM1_3563AttBXhoI_FW | ggggacaagtttgtacaaaaaagcaggcttaCTCGAGATGACAG |
| (SEQ ID NO: 34) | GTCTATCAGCCGG |
| AM1_3563 AttB_RV | ggggaccactttgtacaagaaagctgggtaTTATGACCGCAAGA |
| (SEQ ID NO: 35) | TAAATCGA |
| prSSU_XhoI_RV (SEQ ID NO: 36) | CAACTCGAGGTAGCCTTCTGGCTTGTAGGC |

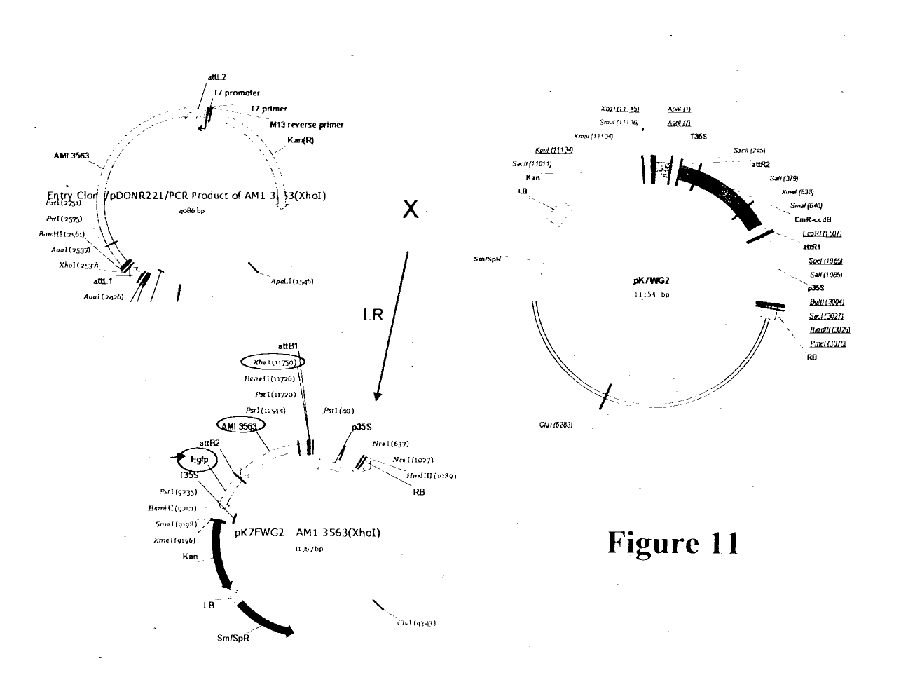

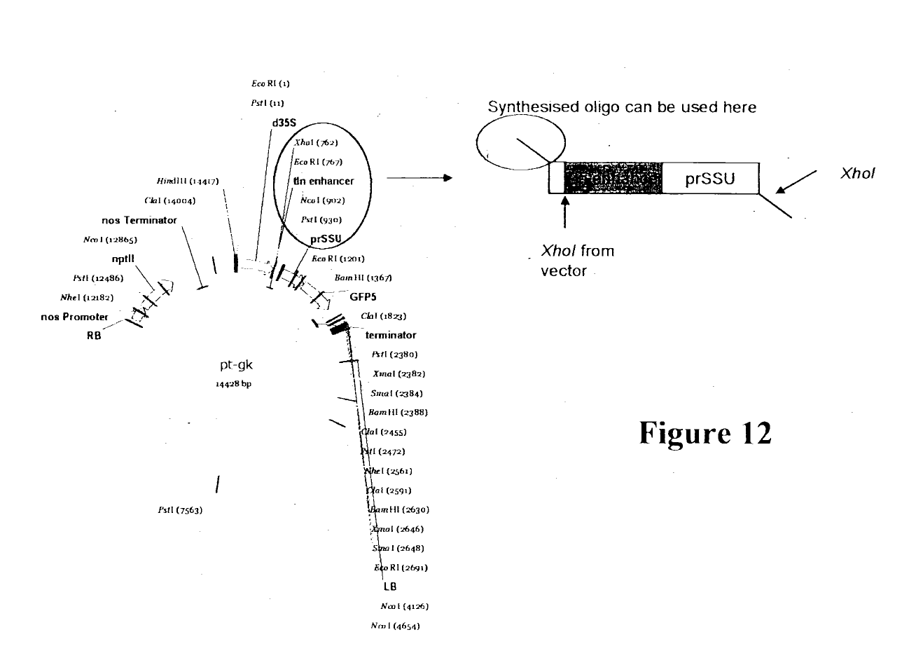

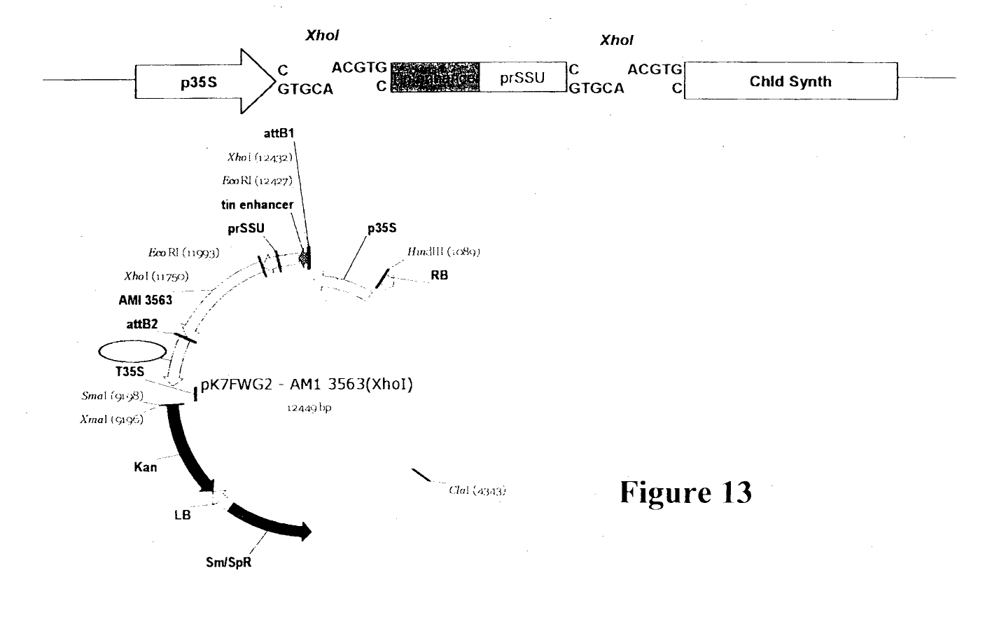

In Table 5, single-underlined and italicised sequences supra are for recombination via attB sites in the vector. The sequences supra shown by double underline are XhoI sites in the amplification primers for amplifying Ch1 d synthase and prSSU sequences. The amplified nucleic acid is introduced into vector pDON221 as described in the legend to FIG. 10, and subsequently used to produce binary plasmid pK7WG2-AMI 3563(XhoI) as described in the legends to FIGS. 11-13. Briefly, the Ch1 d synthase open reading frame is amplified with primers comprising the AttB sites shown in Table 5 for BP recombination, and a unique XhoI restriction site for later insertion of the small subunit of rubisco (prSSU). The Ch1 d synthase gene is then sub-cloned from pDONR to the plant expression vector pK7WG2. The rubisco small subunit-encoding gene (prSSU) is amplified from the vector pt-gk with or without an optional Tin enhancer element. The prSSU in this case encoded the full-length SSU of about 180 amino acids in length. Plasmid pK7WG2-AMI is linearised using XhoI and ligated to the amplified rubisco small subunit-encoding gene (prSSU) with or without an optional Tin enhancer element. Alternatively, the prSSU PCR product is first cloned into a suitable plasmid vector e.g., using TOPO cloning and isolated from the recombinant vector carrying it by endonuclease digestion

Fresh Agrobacterium tumefaciens strain AGL1 or other suitable strain is inoculated into 10 ml LB comprising antibiotic(s) and grown at 28° C. with shaking (−200 rpm) for 2 days according to standard procedures. Then 1 ml of the pre-culture is inoculated into 100 ml of pre-warmed LB (with antibiotics if necessary) in a 500 mL flask. The culture is grown overnight at 28° C. with shaking, to OD550=0.5-0.8 (or OD600=0.3). Cells are harvested and resuspended in 100 mM MgCl₂, incubated for 20 min in ice, collected by centrifugation, and resuspended in 20 mM CaCl₂ for 20 min on ice. Glycerol is added to a final glycerol of 20% (v/v), and cells are snap-frozen in liquid nitrogen and stored at −80° C. until required, or used directly for transformation with 0.1 μg to 1.0 μg binary vector comprising nucleic acid encoding the prSSu-Ch1 d synthase-GFP fusion protein operably under control of the CaMV 35S promoter.

For direct transformation of Agrobacterium tumefaciens, cells from glycerol stocks are mixed with 0.1 μg to 1.0 μg binary vector comprising nucleic acid encoding the prSSu-Ch1 d synthase-GFP fusion protein operably under control of the CaMV 35S promoter, frozen at −80° C., thawed by incubating in a 37° C. water bath for 5 min, and placed on ice for 30 min.

The cells are then spread onto LB plates and cultured for 2 days at 28° C. with the appropriate antibiotic selection e.g., rifampicin 100 μg/ml and/or gentamycin 50 μg/ml and/or kanamycin 50 μg/ml and/or spectinomycin 100 μg/ml and/or streptomycin 300 μg/ml. Then, 10-15 ml LB medium comprising the same appropriate antibiotic selection is inoculated with a colony of cells and the culture grown at 28° C., and 200 rpm for 1-2 days. The cells are harvested and resuspended in infiltration medium e.g., 10 mM MgCl₂, 10 mM MES (pH 5.6), 150 μM acetosyringone, to a final OD600=0.5. Any co-infiltration suspension is prepared at the same concentration and mixed with an equal volume of the cells in filtration medium.

Plant leaf discs, such as from N. tabacum, are transformed by manual injection or biolistics according to standard procedures. For example, a syringe without a needle attached comprising the A. tumefaciens in infiltration medium is applied to the underside of the leaf tissue and pressure is applied to the upper side of the leaf tissue using a syringe nozzle contacting the A. tumefaciens with the plant tissue. Alternatively, a razor or pipette tip coated with the A. tumefaciens is used to prick the surface of the leaf gently, and the Agrobacterium suspension is contacted with the plant tissue for at least about 10 minutes. Alternatively, a vacuum is applied to leaf coated with the A. tumefaciens for at least about 10 minutes.

Leaf discs are analysed for GFP expression after 24-120 hours following transformation to determine transformation efficiency. Those sections of leaf discs expressing GFP are also analysed for Ch1 d synthesis, and/or used to regenerate plantlets according to standard procedures.

### Example 6

### Production of Polyclonal Antibodies Binding to Ch1 d Synthase

This example provides polyclonal antibodies that bind to Ch1 d synthase of A. marina.

### 1. Experimental Methods

Polyclonal antibodies were produced by immunization of rabbits with a multi-epitope immunogen consisting of the amino acid sequence set forth in SEQ ID NO: 37, which is derived from the full-length A. marina amino acid sequence set forth in SEQ ID NO: 12 hereof. Standard protocols for the production of polyclonal antibodies were employed. In summary, the protein immunogen was over-expressed in E. coli with a hexa-histidine tag attached to facilitate subsequent purification using Ni-affinity chromatography. The His-tagged immunogen was purified by Ni-affinity chromatography, and the purified His-tagged synthetic peptide was coupled to KLH or BSA, and used to immunize two New Zealand white rabbits (0.1 mg/ml immunogen concentration). After immunization, the rabbits were sacrificed and the serum was purified by Protein A/G affinity chromatography. Antibodies were titered by standard ELISA. The ELISA titre was defined as the highest dilution of serum providing more than twice the reading of a negative control at OD₄₅₀, and providing an OD₄₅₀ value greater than an assumed background value of 025.

### 2. Results

Two sera were obtained from different bleeds of the same rabbit and processed. These sera were designated D2316-1-1-1R1B1 and D2316-1-1-1R1B2. In ELISA, the antibody designated D2316-1-1-1R1B1 yielded a titer of 1/200 for which the OD₄₅₀ value was 3.18-fold the value obtained for the negative control. The antibody designated D2316-1-1-1R1B2 yielded a titer of 1/800 for which the OD₄₅₀ value was 3.4-fold the value obtained for the negative control.

We claim:

1. A gene construct comprising a Ch1 d synthase gene and one or more origins of replication for maintenance of the gene construct in a cell or chloroplast, wherein said Ch1 d synthase gene comprises a sequence selected from the group consisting of:
(i) the sequence set forth in SEQ ID NO: 11 or a variant thereof comprising a sequence that is degenerate with SEQ ID NO: 11 by virtue of the genetic code and/or that varies from SEQ ID NO: 11 by virtue of a codon usage bias;
(ii) a sequence comprising an open reading frame that encodes the amino acid sequence set forth in SEQ ID NO: 12 or a variant thereof comprising a sequence wherein one or more amino acids of SEQ ID NO: 12 is substituted conservatively for one or more other amino acids in said variant sequence;
(iii) a sequence that is produced by amplification employing one or more primer sequences comprising SEQ ID NO: 14 and SEQ ID NO: 15;
(iv) a sequence having at least about 80% sequence identity to SEQ ID NO: 11;
(v) a sequence comprising an open reading frame that encodes an amino acid sequence having at least about 80% identity to the amino acid sequence set forth in SEQ ID NO: 12;
(vi) a sequence that is produced by amplification employing one or more primer sequences comprising SEQ ID NO: 14 and SEQ ID NO: 15;
(vii) a sequence that is produced by amplification employing primer sequences comprising SEQ ID NO: 18 and SEQ ID NO: 20 or primer sequences comprising SEQ ID NO: 19 and SEQ ID NO: 20;
(viii) a sequence that hybridizes under at least moderate stringency conditions to a sequence that is complementary to (i) or (ii) or fragment thereof comprising at least 10 nucleotides in length; and
(ix) a sequence that is complementary to a sequence at (i) or (ii) or (iii) or (iv) or (v) or (vi) or (vii) or (viii).

2. The gene construct of claim 1 comprising the sequence set forth in SEQ ID NO: 11 or a variant thereof comprising a sequence that is degenerate with SEQ ID NO: 11 by virtue of the genetic code and/or that varies from SEQ ID NO: 11 by virtue of a codon usage bias or having at least 80% identity to SEQ ID NO: 11.

3. (canceled)

4. The gene construct of claim 1 comprising a sequence that encodes the amino acid sequence set forth in SEQ ID NO: 12 or a variant thereof comprising a sequence wherein one or more amino acids of SEQ ID NO: 12 is substituted conservatively for one or more other amino acids in said variant sequence or having at least 80% identity to SEQ ID NO: 12.

5. (canceled)

6. The gene construct of claim 1, wherein the origin of replication is operable in a prokaryote.

7-8. (canceled)

9. The gene construct of claim 1, wherein the origin of replication is operable in a eukaryote.

10-12. (canceled)

13. The gene construct of claim 1, wherein the origin of replication is operable in a chloroplast.

14. The gene construct of claim 1, comprising one or more additional elements selected from a selectable marker gene, a 5′ non-coding region, a 3′ non-coding region, a sequence encoding a targeting peptide or transit peptide, a sequence encoding a detectable label, a recombinase site sequence, a restriction endonuclease cleavage site and a multiple cloning site.

15-23. (canceled)

24. The gene construct of claim 1, wherein said gene construct is an expression construct that provides for transcription of mRNA encoding a functional Ch1 d synthase polypeptide or a functional fragment thereof in the nucleus or chloroplast of a eukaryote and/or in a prokaryotic cell.

24. The gene construct of claim 1, wherein said gene construct is an expression construct that provides for translation of a functional Ch1 d synthase polypeptide or a functional fragment thereof in a chloroplast or prokaryotic cell.

25. The gene construct of claim 1, wherein said gene construct is a shuttle vector that is able to be maintained and/or replicate in at least two different host organisms.

26. The gene construct of claim 1, wherein said gene construct is a binary plasmid.

27. A method for producing a gene construct, said method comprising linking a promoter or active fragment or derivative thereof to a Ch1 d synthase gene such that the promoter confers Ch1 d synthase expression or a pattern of Ch1 d synthase expression on said gene in a cell or chloroplast, wherein said Ch1 d synthase gene comprises a sequence selected from the group consisting of:
(i) the sequence set forth in SEQ ID NO: 11 or a variant thereof comprising a sequence that is degenerate with SEQ ID NO: 11 by virtue of the genetic code and/or that varies from SEQ ID NO: 11 by virtue of a codon usage bias;
(ii) a sequence comprising an open reading frame that encodes the amino acid sequence set forth in SEQ ID NO: 12 or a variant thereof comprising a sequence wherein one or more amino acids of SEQ ID NO: 12 is substituted conservatively for one or more other amino acids in said variant sequence;
(iii) a sequence that is produced by amplification employing one or more primer sequences comprising SEQ ID NO: 14 and SEQ ID NO: 15;
(iv) a sequence having at least about 80% sequence identity to SEQ ID NO: 11;
(v) a sequence comprising an open reading frame that encodes an amino acid sequence having at least about 80% identity to the amino acid sequence set forth in SEQ ID NO: 12;
(vi) a sequence that is produced by amplification employing one or more primer sequences comprising SEQ ID NO: 14 and SEQ ID NO: 15;
(vii) a sequence that is produced by amplification employing primer sequences comprising SEQ ID NO: 18 and SEQ ID NO: 20 or primer sequences comprising SEQ ID NO: 19 and SEQ ID NO: 20; and
(viii) a sequence that hybridizes under at least moderate stringency conditions to a sequence that is complementary to (i) or (ii) or fragment thereof comprising at least 10 nucleotides in length.

28-29. (canceled)

30. A cell comprising the gene construct of claim 1 or a fragment of said gene construct comprising a Ch1 d synthase gene.

31-32. (canceled)

33. A chloroplast comprising a gene construct of claim 1 or a fragment of said gene construct comprising a Ch1 d synthase gene.

34. (canceled)

35. A plant tissue, organ or whole plant comprising a gene construct of claim 1 or a fragment of said gene construct comprising a Ch1 d synthase gene.

36-37. (canceled)

38. A method for producing a transgenic chloroplast or cell, said method comprising introducing the gene construct of claim 1 or a fragment thereof comprising the Ch1 d synthase gene of said gene construct into a chloroplast or cell, thereby producing a transgenic chloroplast or cell.

39-42. (canceled)

43. A method for producing a transgenic plant or plantlet, said method comprising:
(i) providing, producing or obtaining a transgenic plant cell or callus comprising the gene construct of claim 1 or a fragment thereof comprising the Ch1 d synthase gene of said gene construct; and
(ii) regenerating a transgenic plant or plantlet from the transgenic plant cell or callus at (i), thereby producing a transgenic plant or plantlet.

44. A method for producing a transgenic plant or plantlet, said method comprising:
(i) providing, producing or obtaining a transgenic chloroplast comprising the gene construct of claim 1 or a fragment thereof comprising the Ch1 d synthase gene of said gene construct;
(ii) producing a transgenic plant cell or callus comprising the transgenic chloroplast at (i); and
(iii) regenerating a transgenic plant or plantlet from a cell or callus at (ii), thereby producing a transgenic plant or plantlet.

45-48. (canceled)

49. A method for breeding a transgenic plant, said method comprising:
(i) providing, producing or obtaining a transgenic plant comprising the gene construct of claim 1 or a fragment thereof comprising the Ch1 d synthase gene of said gene construct;
(ii) breeding the transgenic plant produced at (i) to thereby produce a zygote comprising the gene construct or fragment thereof comprising a Ch1 d synthase gene; and
(iii) developing the zygote into a whole plant comprising a Ch1 d synthase gene.

50. A method for breeding a transgenic plant, said method comprising:
(i) providing, producing or obtaining plant reproductive material comprising the gene construct of claim 1 or a fragment thereof comprising the Ch1 d synthase gene of said gene construct;
(ii) combining reproductive material of a plant with the reproductive material at (i) such that a zygote comprising the gene construct or fragment is produced; and
(iii) developing the zygote into a whole plant comprising a Ch1 d synthase gene.

51. A method comprising:
(i) providing, producing or obtaining a transgenic plant, plantlet or plant part comprising the gene construct of claim 1 or a fragment thereof comprising the Ch1 d synthase gene of said gene construct; and
(ii) maintaining the transgenic plant, plantlet or plant part for a time and under conditions sufficient for the plant to reproduce vegetatively.

52-71. (canceled)

72. A method of producing Ch1 d in a chloroplast, cell, plant part or plant that normally produces Ch1 a in the absence of Ch1 d, said method comprising expressing a Ch1 d synthase gene in a chloroplast, cell, plant part or plant for a time and under conditions sufficient for Ch1 d to be produced from endogenous Ch1 a in the chloroplast, cell, plant part or plant, wherein said Ch1 d synthase gene comprises a sequence selected from the group consisting of:
(i) the sequence set forth in SEQ ID NO: 11 or a variant thereof comprising a sequence that is degenerate with SEQ ID NO: 11 by virtue of the genetic code and/or that varies from SEQ ID NO: 11 by virtue of a codon usage bias;
(ii) a sequence comprising an open reading frame that encodes the amino acid sequence set forth in SEQ ID NO: 12 or a variant thereof comprising a sequence wherein one or more amino acids of SEQ ID NO: 12 is substituted conservatively for one or more other amino acids in said variant sequence;
(iii) a sequence that is produced by amplification employing one or more primer sequences comprising SEQ ID NO: 14 and SEQ ID NO: 15;
(iv) a sequence having at least about 80% sequence identity to SEQ ID NO: 11;
(v) a sequence comprising an open reading frame that encodes an amino acid sequence having at least about 80% identity to the amino acid sequence set forth in SEQ ID NO: 12;
(vi) a sequence that is produced by amplification employing one or more primer sequences comprising SEQ ID NO: 14 and SEQ ID NO: 15;
(vii) a sequence that is produced by amplification employing primer sequences comprising SEQ ID NO: 18 and SEQ ID NO: 20 or primer sequences comprising SEQ ID NO: 19 and SEQ ID NO: 20;
(viii) a sequence that hybridizes under at least moderate stringency conditions to a sequence that is complementary to (i) or (ii) or fragment thereof comprising at least 10 nucleotides in length; and
(ix) a sequence that is complementary to a sequence at (i) or (ii) or (iii) or (iv) or (v) or (vi) or (vii) or (viii).

73-74. (canceled)

75. A method of producing Ch1 d in a chloroplast, cell, plant part or plant that does not normally produce a chlorophyll pigment, said method comprising expressing a Ch1 d synthase gene in a chloroplast, cell, plant part or plant in the presence of an amount of exogenous Ch1 a sufficient for Ch1 d to be produced in the chloroplast, cell, plant part or plant, wherein said Ch1 d synthase gene comprises a sequence selected from the group consisting of:
(i) the sequence set forth in SEQ ID NO: 11 or a variant thereof comprising a sequence that is degenerate with SEQ ID NO: 11 by virtue of the genetic code and/or that varies from SEQ ID NO: 11 by virtue of a codon usage bias;
(ii) a sequence comprising an open reading frame that encodes the amino acid sequence set forth in SEQ ID NO: 12 or a variant thereof comprising a sequence wherein one or more amino acids of SEQ ID NO: 12 is substituted conservatively for one or more other amino acids in said variant sequence;
(iii) a sequence that is produced by amplification employing one or more primer sequences comprising SEQ ID NO: 14 and SEQ ID NO: 15;
(iv) a sequence having at least about 80% sequence identity to SEQ ID NO: 11;
(v) a sequence comprising an open reading frame that encodes an amino acid sequence having at least about 80% identity to the amino acid sequence set forth in SEQ ID NO: 12;
(vi) a sequence that is produced by amplification employing one or more primer sequences comprising SEQ ID NO: 14 and SEQ ID NO: 15;
(vii) a sequence that is produced by amplification employing primer sequences comprising SEQ ID NO: 18 and SEQ ID NO: 20 or primer sequences comprising SEQ ID NO: 19 and SEQ ID NO: 20;
(viii) a sequence that hybridizes under at least moderate stringency conditions to a sequence that is complementary to (i) or (ii) or fragment thereof comprising at least 10 nucleotides in length; and
(ix) a sequence that is complementary to a sequence at (i) or (ii) or (iii) or (iv) or (v) or (vi) or (vii) or (viii).

76-78. (canceled)

79. An antibody that binds to Ch1 d synthase polypeptide or a fragment thereof.

80-83. (canceled)


SEQUENCE LISTING (37 sequences)

SEQ ID NO: 1 - DNA, 23 bp, Artificial Sequence
  synthetic oligonucleotide forward primer for AM1_0606
atgagcgatttcagcagctcagt

SEQ ID NO: 2 - DNA, 27 bp, Artificial Sequence
  synthetic oligonucleotide reverse primer for AM1_0606
gcaaggagcaggctgagaatatcttgt

SEQ ID NO: 3 - DNA, 23 bp, Artificial Sequence
  synthetic oligonucleotide forward primer for AM1_0824
ttagccactgctgacctcaatga

SEQ ID NO: 4 - DNA, 23 bp, Artificial Sequence
  synthetic oligonucleotide reverse primer for AM1_0824
tggctgaaaggcttgaggattcg

SEQ ID NO: 5 - DNA, 22 bp, Artificial Sequence
  synthetic oligonucleotide forward primer for AM1_3563
ttggcaggtgctaggagaacag

SEQ ID NO: 6 - DNA, 24 bp, Artificial Sequence
  synthetic oligonucleotide reverse primer for AM1_3563
aaggatgactatctgtcaggatgg

SEQ ID NO: 7 - DNA, 26 bp, Artificial Sequence
  synthetic oligonucleotide forward primer for AM1_4161
ctgtggtgttagacggtgtgctctat

SEQ ID NO: 8 - DNA, 22 bp, Artificial Sequence
  synthetic oligonucleotide reverse primer for AM1_4161
aaactggcgaagcagatggctg

SEQ ID NO: 9 - DNA, 24 bp, Artificial Sequence
  synthetic oligonucleotide forward primer for AM1_5780
acagccttgacttgggcaatgtac

SEQ ID NO: 10 - DNA, 25 bp, Artificial Sequence
  synthetic oligonucleotide reverse primer for AM1_5780
atcgagctgatagccacatagctcc

SEQ ID NO: 11 - DNA, 1533 bp, Acaryochloris marina
  CDS (1)..(1530)
atgacaggtctatcagccggatccctcactgcaggactgcacaataccctcaaacagctagaggccaaaccccgacagtgggtagaatcaaagttacagtcctattcccagaccttatttcagcggatctttagcaaacagacccaagcattcagccagattccaggccctcagcctctattccccattggtaatgtctgggctctgcagcaagggaaacctatttggcaggtgctaggagaacaggggcagcaatatggtggcttgtccttattctggattttggcccggccatccttagttctcaacgaccccgatttaatccaacaagtcctgctcacggcccagcagcagactaacccacattcccatgaacatcgggccaaccaagctgatttttataaagatatcccccgcaaagcccttcgccccatcctgacagatagtcatccttttgtagccaaaactgctggatcgaaatggcagcatctggtcaaaaataatccatttaatatgacctacttccgcgagtggatggagactcaaattcagcccttgcaggtttttatcgaaagtcgggcggaggcattggtcaaacagtctgagaccgaaatgctccccgcttatgaggtcattcagaagctcgccttcgacggcttttccctggcaaccgtaggccaagtctttcccgatcatgtctttgaacaattcaataccctctgtgcaacgggcactgatcgcatgaaccgatcctccattgccaactggatgattccggaagaacccaaaggtaagcattataaagaagcaagtcgccaatggtttcaacgctttagcgaagtgctagaaactactcaagccgatcctgctggtggttcgctcttggattgggttctgaaacgaggaggctctgcctttacccctgaacaaatgcgaaacttctgcgctggtgtctatccaggcggagcggtttctgcgcccagcggtattacctctacacttcatttgctatttcagcatcctcaagccctatcagcgcttcaacacgatttggacgggttattcaaagaacccctcaccttggaacgactggaaaactgtatcccagtggaacaagtattgcgagagtccctgcgactctggccgcccgtgcccttttttacgcgcaatacggttggcgctacccatctagctggtcacgagattcccgcgaatacccaaatttttatcaataactggtttctccatcgcctctcaccccactggcacgattcagactcttttcagccagaacgctgggataccaaaacgttggccgaaaatgactatggtagcgattactttttcccgtttggtcgaggtgagcgagcttgcattggtcaagattttgccaaattctttatgcgcttaaccttagccaccttgttagcaaaagtagagatcgaatttggcgatcagccccatgaccaagaattttattttggcgtctctgtgcctagacagctaaaagctcgatttatcttgcggtcataa

SEQ ID NO: 12 - protein, 510 aa, Acaryochloris marina
MTGLSAGSLTAGLHNTLKQLEAKPRQWVESKLQSYSQTLFQRIFSKQTQAFSQIPGPQPLFPIGNVWALQQGKPIWQVLGEQGQQYGGLSLFWILARPSLVLNDPDLIQQVLLTAQQQTNPHSHEHRANQADFYKDIPRKALRPILTDSHPFVAKTAGSKWQHLVKNNPFNMTYFREWMETQIQPLQVFIESRAEALVKQSETEMLPAYEVIQKLAFDGFSLATVGQVFPDHVFEQFNTLCATGTDRMNRSSIANWMIPEEPKGKHYKEASRQWFQRFSEVLETTQADPAGGSLLDWVLKRGGSAFTPEQMRNFCAGVYPGGAVSAPSGITSTLHLLFQHPQALSALQHDLDGLFKEPLTLERLENCIPVEQVLRESLRLWPPVPFFTRNTVGATHLAGHEIPANTQIFINNWFLHRLSPHWHDSDSFQPERWDTKTLAENDYGSDYFFPFGRGERACIGQDFAKFFMRLTLATLLAKVEIEFGDQPHDQEFYFGVSVPRQLKARFILRS

SEQ ID NO: 13 - DNA, 230 bp, Artificial Sequence
  synthetic oligonucleotide comprising a open reading frame fragment of A. marina Chl d synthase gene
ttggcaggtgctaggagaacaggggcagcaatatggtggcttgtccttattctggattttggcccggccatccttagttctcaacgaccccgatttaatccaacaagtcctgctcacggcccagcagcagactaacccacattcccatgaacatcgggccaaccaagctgatttttataaagatatcccccgcaaagcccttcgccccatcctgacagatagtcatcctt

SEQ ID NO: 14 - DNA, 8 bp, Artificial Sequence
  synthetic oligonucleotide primer/probe for amplifying full-length open reading frame of the A. marina Chl d synthase-encoding gene
atgacagg

SEQ ID NO: 15 - DNA, 8 bp, Artificial Sequence
  synthetic oligonucleotide primer for amplifying full-length open reading frame of the A. marina Chl d synthase-encoding gene
ttatgacc

SEQ ID NO: 16 - DNA, 12 bp, Artificial Sequence
  sythetic oligonucleotide 12-mer primer
atgacaggtcta

SEQ ID NO: 17 - DNA, 12 bp, Artificial Sequence
  sythetic oligonucleotide 12-mer primer
ttatgaccgcaa

SEQ ID NO: 18 - DNA, 35 bp, Artificial Sequence
  synthetic oligonucleotide forward primer for amplifying AM1_3563
cgcgcggcagccatatgacaggtctatcagccgga

SEQ ID NO: 19 - DNA, 20 bp, Artificial Sequence
  synthetic oligonucleotide forward primer for amplifying AM1_3563
gggaaacctatttggcaggt

SEQ ID NO: 20 - DNA, 39 bp, Artificial Sequence
  synthetic oligonucleotide reverse primer for amplifying AM1_3563
gttagcagccggatccttatgaccgcaagataaatcgag

SEQ ID NO: 21 - DNA, 1518 bp, Acaryochloris marina
  CDS (1)..(1518)
atgacaggtctatcagccggatccctcactgcaggactgcacaataccctcaaacagctagaggccaaaccccgacagtgggtagaatcaaagttacagtcctattcccagaccttatttcagcggatctttagcaaacagacccaagcattcagccagattccaggccctcagcctctattccccattggtaatgtctgggctctgcagcaagggaaacctatttggcaggtgctaggagaacaggggcagcaatatggtggcttgtccttattctggattttggcccggccatccttagttctcaacgaccccgatttaatccaacaagtcctgctcacggcccagcagcagactaacccacattcccatgaacatcgggccaaccaagctgatttttataaagatatcccccgcaaagcccttcgccccatcctgacagatagtcatccttttgtagccaaaactgctggatcgaaatggcagcatctggtcaaaaataatccatttaatatgacctacttccgcgagtggatggagactcaaattcagcccttgcaggtttttatcgaaagtcgggcggaggcattggtcaaacagtctgagaccgaaatgctccccgcttatgaggtcattcagaagctcgccttcgacggcttttccctggcaaccgtaggccaagtctttcccgatcatgtctttgaacaattcaataccctctgtgcaacgggcactgatcgcatgaaccgatcctccattgccaactggatgattccggaagaacccaaaggtaagcattataaagaagcaagtcgccaatggtttcaacgctttagcgaagtgctagaaactactcaagccgatcctgctggtggttcgctcttggattgggttctgaaacgaggaggctctgcctttacccctgaacaaatgcgaaacttctgcgctggtgtctatccaggcggagcggtttctgcgcccagcggtattacctctacacttcatttgctatttcagcatcctcaagccctatcagcgcttcaacacgatttggacgggttattcaaagaacccctcaccttggaacgactggaaaactgtatcccagtggaacaagtattgcgagagtccctgcgactctggccgcccgtgcccttttttacgcgcaatacggttggcgctacccatctagctggtcacgagattcccgcgaatacccaaatttttatcaataactggtttctccatcgcctctcaccccactggcacgattcagactcttttcagccagaacgctgggataccaaaacgttggccgaaaatgactatggtagcgattactttttcccgtttggtcgaggtgagcgagcttgcattggtcaagattttgccaaattctttatgcgcttaaccttagccaccttgttagcaaaagtagagatcgaatttggcgatcagccccatgaccaagaattttattttggcgtctctgtgcctagacagctaaaagctcgattt

SEQ ID NO: 22 - protein, 506 aa, Acaryochloris marina
MTGLSAGSLTAGLHNTLKQLEAKPRQWVESKLQSYSQTLFQRIFSKQTQAFSQIPGPQPLFPIGNVWALQQGKPIWQVLGEQGQQYGGLSLFWILARPSLVLNDPDLIQQVLLTAQQQTNPHSHEHRANQADFYKDIPRKALRPILTDSHPFVAKTAGSKWQHLVKNNPFNMTYFREWMETQIQPLQVFIESRAEALVKQSETEMLPAYEVIQKLAFDGFSLATVGQVFPDHVFEQFNTLCATGTDRMNRSSIANWMIPEEPKGKHYKEASRQWFQRFSEVLETTQADPAGGSLLDWVLKRGGSAFTPEQMRNFCAGVYPGGAVSAPSGITSTLHLLFQHPQALSALQHDLDGLFKEPLTLERLENCIPVEQVLRESLRLWPPVPFFTRNTVGATHLAGHEIPANTQIFINNWFLHRLSPHWHDSDSFQPERWDTKTLAENDYGSDYFFPFGRGERACIGQDFAKFFMRLTLATLLAKVEIEFGDQPHDQEFYFGVSVPRQLKARF

SEQ ID NO: 23 - DNA, 1533 bp, Acaryochloris marina
  CDS (1)..(1530)
atgacaggtctatcagccggatccctcactgcaggactgcacaataccctcaaacagatagaggccaaaccccggcagtgggtggagtcaaagttacagtcctattcccaggccttatttcagcggatctttagcaaacaaacccaagcattcggccagattccaggccctcagcctctattccccattggtaatgtctgggctctgcagcaaggaaaaccgatttggcaggtgctaggagaacaggggcagcaatatggtggcctgtccttattctggattttggcccggccatccttagttctcaacgaccccgatttaatccaacaagtcttgctcacggcccagcagcagactaacccacactcccatgaacatcgggccacccaagctgatttttataaagatatccctcgcaaagccctccgccccatcctgacggatagtcatccttttgtagccaaaactgctggatcgaaatggcagcatctggttaaaaataatccatttaatatgacctacttccgcgagtggatggagactcaaattcagcccttgcaggtttttatcgaaagtcgggcggaggtattggtcaaacagtctgagaccgaaatgctcccggcgtatgaggtcattcagaagctcgctttcgacggcttttctctggcaaccgtaggccaagtctttcccgatcacgtctttgaacaattcaataccctctgtgcaacaggcactgatcgcatgaaccgatcctccattgccaactggatgattccggaagaacccaaaggtaagcactataaagaagcaagtcgccaatggtttcaacgctttagcgaagtgctagaaactactcaagccgatcctgctggtggttcgctcttggattgggttctgaaacgaggaggctctgcctttacccctgaacaaatgcgaaacttctgcgctggtgtctatccaggcggagcggtttctgcgcccagcggtattacctctacacttcatttgctatttcagtatcctcaagccctatcagcgcttcaacacgatttggacgggttattcaaagaacccctcaccttggaacgactggaaaactgtatcccagtggagcaagtattgcgagagtccctgcgactctggccgcctgtgcccttttttacgcgcaatacggttggcgctacccatctagctggtcacgagattcccgcgaatacccaaatttttatcaataactggtttctccatcgcctctcaccccactggcacgattcagactcttttcagccagaacgctgggatgccaaaacgttggccgaaaatgactatggtagcgattactttttcccgtttggtcgaggggatcgagcttgcattggtcaagattttgccaaattctttatgcgcttaaccttagctaccttgttagcaaaagtagagatcgaatttggcgatcagccccatgatcaagaattttactttggcgtctctgtgcctagacagctaaaagctcgatttatcttgcggtcataa

SEQ ID NO: 24 - protein, 510 aa, Acaryochloris marina
MTGLSAGSLTAGLHNTLKQIEAKPRQWVESKLQSYSQALFQRIFSKQTQAFGQIPGPQPLFPIGNVWALQQGKPIWQVLGEQGQQYGGLSLFWILARPSLVLNDPDLIQQVLLTAQQQTNPHSHEHRATQADFYKDIPRKALRPILTDSHPFVAKTAGSKWQHLVKNNPFNMTYFREWMETQIQPLQVFIESRAEVLVKQSETEMLPAYEVIQKLAFDGFSLATVGQVFPDHVFEQFNTLCATGTDRMNRSSIANWMIPEEPKGKHYKEASRQWFQRFSEVLETTQADPAGGSLLDWVLKRGGSAFTPEQMRNFCAGVYPGGAVSAPSGITSTLHLLFQYPQALSALQHDLDGLFKEPLTLERLENCIPVEQVLRESLRLWPPVPFFTRNTVGATHLAGHEIPANTQIFINNWFLHRLSPHWHDSDSFQPERWDAKTLAENDYGSDYFFPFGRGDRACIGQDFAKFFMRLTLATLLAKVEIEFGDQPHDQEFYFGVSVPRQLKARFILRS

SEQ ID NO: 25 - DNA, 1326 bp, Acaryochloris marina
  CDS (1)..(1323)
gggaaacctatttggcaggttttaggagagtatgggcatcagtatggtggcttatccttattctggatttttgccaagccgtctttggttctcaatgatccagacttaatccaacaagtgttgctgaccgcgctgcagcagacaggcccaaaatctagtggacatcgggccactcaagctgacttttataaagatctaccgcgcaaggcattacggccaatactgacggatagccatccctttattgctaaagatactggacctaaatggaagcatctggttaaaaataatccctttaatatgacctatttccgcgaatggctggagactcaagttcagcccttgcaagcgtttgtagaaactcgtgcagaagagtgggtcaggcagtctgaaacagaaatgcttcctgcctatgaggtgattcagaagttagcgtttgatggattctcgttggcaacggtgggccaagtgttccccaaacaggtttttgagcagtttaataccctatgtgcaacggggacagatcggatgaatcgatcgtctattgccagttggctgatttctgaagaaccgaaaggcaaacattatcaagacgtaagtcgccaatggtttcgactatttggtgaagtggttgaaactgcccaagccaatccttcgggtggatcgttactggattgggtgctgaagcgagggggatctgcttttacgccagaacaaatgcgtaatttttgtgccggtgtctatccaggaggggctgtttctgcaccgagcggcattacgtctaccctgcatttactgtcacaacatcctcaggcattatctgctctccaacaagagcttgaggttttatgtcgtgagcctttaacccttgagcggctcgaaagctttgtgcctgtggaacaagtgttgcgagaatcgctacggctatggcctccggtcccctttttcacgcgcaatacgatgtgcgatcgcatcgttaatcttgcgggtcatgacatcccagccaatacccaaattttcatcaccaactggtttctacatcggctctctccccactggcaggattcagagtcctttaagccagagcgttgggatgccaagacgatggcagagaatgactatggcagtgattacttcttcccgttcggtcgtggcaatcgggcttgcattggccaagactttgccaagtttttcatgcgtctaacagtggcaactctattagccaaagtagagattcaatttggagatcagccacatgatcaagacttttactttggggtctcggtgcctagacaattaaaagctcgatttatcttgcggtcataa

SEQ ID NO: 26 - protein, 441 aa, Acaryochloris marina
GKPIWQVLGEYGHQYGGLSLFWIFAKPSLVLNDPDLIQQVLLTALQQTGPKSSGHRATQADFYKDLPRKALRPILTDSHPFIAKDTGPKWKHLVKNNPFNMTYFREWLETQVQPLQAFVETRAEEWVRQSETEMLPAYEVIQKLAFDGFSLATVGQVFPKQVFEQFNTLCATGTDRMNRSSIASWLISEEPKGKHYQDVSRQWFRLFGEVVETAQANPSGGSLLDWVLKRGGSAFTPEQMRNFCAGVYPGGAVSAPSGITSTLHLLSQHPQALSALQQELEVLCREPLTLERLESFVPVEQVLRESLRLWPPVPFFTRNTMCDRIVNLAGHDIPANTQIFITNWFLHRLSPHWQDSESFKPERWDAKTMAENDYGSDYFFPFGRGNRACIGQDFAKFFMRLTVATLLAKVEIQFGDQPHDQDFYFGVSVPRQLKARFILRS

SEQ ID NO: 27 - DNA, 1533 bp, Acaryochloris marina
  CDS (1)..(1530)
atgacaggtctatcagccggatccctcactacaggactgcacaataccctcaaacagatagaggccaaaccccgacagtgggtagagtcaaagttacagtcctattcccaggccttatttcagcggatctttagcaaacagacccaagcattcagccagattccaggccctcagcctctcttccccattggtaatgtctgggttctgcagcaagggaaaccgatttggcaggtgctaggagaacaggggcagcaatatggtggcttgtccttattctggattttggcccggccatccttagttctcaacgaccccgatttaatccaacaagtcctgcttacggcccagcagcagactaacccacattcccatgaacatcgggccaaccaagctgatttttataaagatatcccccgcaaagcccttcgccccatcctcacggatagtcatccttttgtggccaaaactgctggatcgaaatggcagcatctggttaaaaataatccatttaatatgacctacttccgcgagtggatggagactcaaattcagcccttgcaggtttttatcgaaagtcgggcggaggcattggtcaaacagtctgagaccgaaatgctcccagcttatgaggtcattcagaaactagtcttcgacagtttttccctggcaaccgtaggccaagtctttcccgatcaggtctttgaacagttcaataccctctgtgcaacaggcactgatcgcatgaaccgatcctccattgccagctggatgattccggaagaaccaaaaggtaagcactataaagaagcaagtcgccaatggtttcagcgctttagcgaagtgctagaaaccactcaagccgatcctgctggtggttcgctcctggattgggttctgaaacgaggaggctctgcctttacccctgaacaaatgcgaaacttctgcgctggggtctacccaggcggagcggtttctgcacccagcggtattacctctacacttcatttgctgtttcagcatcctcaagccctatcggcgcttcagcacgatttggacgggttattcaaggaacccctcaccttggaacgactggaaaattgtctcccagtggagcaagtattgcgagagtccctgcgactctggccgcccgtgcccttttttacgcgcaatacggttggcgctacccatctagcgggtcacgagattcccgcgaatacccaaatttttatcaataactggtttctccatcgcctctcaccccactggcacgattcagactcttttcagccagaacgctgggatgccaaaacgttggccgaaaatgactatggtagcgattactttttcccgtttggtcgaggtgatcgagcttgcattggtcaagattttgccaaattctttatgcgcttaaccttagccaccttgttagcaaaagtagagatcgaatttggcgatcagccccatgaccaagaattttattttggcgtctctgtgcctagacagctaaaagctcgatttatcttgcggtcataa

SEQ ID NO: 28 - protein, 510 aa, Acaryochloris marina
MTGLSAGSLTTGLHNTLKQIEAKPRQWVESKLQSYSQALFQRIFSKQTQAFSQIPGPQPLFPIGNVWVLQQGKPIWQVLGEQGQQYGGLSLFWILARPSLVLNDPDLIQQVLLTAQQQTNPHSHEHRANQADFYKDIPRKALRPILTDSHPFVAKTAGSKWQHLVKNNPFNMTYFREWMETQIQPLQVFIESRAEALVKQSETEMLPAYEVIQKLVFDSFSLATVGQVFPDQVFEQFNTLCATGTDRMNRSSIASWMIPEEPKGKHYKEASRQWFQRFSEVLETTQADPAGGSLLDWVLKRGGSAFTPEQMRNFCAGVYPGGAVSAPSGITSTLHLLFQHPQALSALQHDLDGLFKEPLTLERLENCLPVEQVLRESLRLWPPVPFFTRNTVGATHLAGHEIPANTQIFINNWFLHRLSPHWHDSDSFQPERWDAKTLAENDYGSDYFFPFGRGDRACIGQDFAKFFMRLTLATLLAKVEIEFGDQPHDQEFYFGVSVPRQLKARFILRS

SEQ ID NO: 29 - DNA, 37 bp, Artificial Sequence
  synthetic oligonucleotide forward primer for amplifying AM1_3563R1
ggagaaccacattaaaatgacaggtctatcagccgga

SEQ ID NO: 30 - DNA, 39 bp, Artificial Sequence
  synthetic oligonucleotide reverse primer for amplifying AM1_3563
gctgactcataccaggttatgaccgcaagataaatcgag

SEQ ID NO: 31 - DNA, 32 bp, Artificial Sequence
  synthetic oligonucleotide forward primer for amplifying AM1_3563R2
atggggcgattcaggagaaccacattaaaatg

SEQ ID NO: 32 - DNA, 1288 bp, Artificial Sequence
  kanamycin gene cassette of plasmid pBS-HO2-Kan
gaattccccggatccgtcgacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtcgacggatccggggaattc

SEQ ID NO: 33 - DNA, 51 bp, Artificial Sequence
  synthetic oligonucleotide forward primer for amplifying AMI_3563 AttB
ggggacaagtttgtacaaaaaagcaggcttaatgacaggtctatcagccgg

SEQ ID NO: 34 - DNA, 57 bp, Artificial Sequence
  synthetic oligonucleotide forward primer for amplifying AMI_3563 AttBXhoI
ggggacaagtttgtacaaaaaagcaggcttactcgagatgacaggtctatcagccgg

SEQ ID NO: 35 - DNA, 52 bp, Artificial Sequence
  synthetic oligonucleotide reverse primer/probe for amplifying AMI_3563
ggggaccactttgtacaagaaagctgggtattatgaccgcaagataaatcga

SEQ ID NO: 36 - DNA, 30 bp, Artificial Sequence
  synthetic oligonucleotide reverse primer for amplifying prSSU_XhoI
caactcgaggtagccttctggcttgtaggc

SEQ ID NO: 37 - protein, 60 aa, Artificial Sequence
  Multi-epitope peptide for antibody production
LEAKPRQWVERANQADFYKDEEPKGKHYKEKEPLTLERLEDSFQPERWDTQLKARFILRS